SEQUENCE LISTING (11 sequences)

SEQ ID NO: 1 - DNA, 203 bp, synthetic construct
  source 1..203: mol_type = other DNA
catgcttcttgccccttgggcctccccccagcccctcctccccttcctgcacccgtacccccgtggtctttgaataaagtctgagtgggcggcagcggccgggagaatcggagtgctagaggatgaccttaaatcagggacgatattataattgactaagatatctgtaggtagaaggtataaacgaccagccacacacttcc

SEQ ID NO: 2 - DNA, 58 bp, synthetic construct
  source 1..58: mol_type = other DNA
ttatttcagactcacccgccgtcgccaaaactcttagcctcacgatctcctactggaa

SEQ ID NO: 3 - DNA, 58 bp, synthetic construct
  source 1..58: mol_type = other DNA
ttatttcagactcacccgccgtcgcaaaaaatcttagcctcacgatctcctactggaa

SEQ ID NO: 4 - DNA, 130 bp, synthetic construct
  source 1..130: mol_type = other DNA
aggaggggaaggacgtgggcatgggggcaccagaaacttatttcagactcacccgccgtcgccgaacctcttagcctcacgatctcctactggaatttagtccctgctataatattaactgattctatag

SEQ ID NO: 5 - DNA, 130 bp, synthetic construct
  source 1..130: mol_type = other DNA
aggaggggaaggacgtgggcatgggggcaccagaaacttatttcagactcacccgccgtcgccaaaactcttagcctcacgatctcctactggaatttagtccctgctataatattaactgattctatag

SEQ ID NO: 6 - DNA, 130 bp, synthetic construct
  source 1..130: mol_type = other DNA
aggaggggaaggacgtgggcatgggggcaccagaaacttatttcagactcacccgccgtcgccggccctcttagcctcacgatctcctactggaatttagtccctgctataatattaactgattctatag

SEQ ID NO: 7 - DNA, 200 bp, synthetic construct
  source 1..200: mol_type = other DNA
gtacgaagaacggggaacccggaggggggtcggggaggaggggaaggacgtgggcatgggggcaccagaaacttatttcagactcacccgccgtcgccaaaactcttagcctcacgatctcctactggaatttagtccctgctataatattaactgattctatagacatccatcttccatatttgctggtcggtgtgtga

SEQ ID NO: 8 - RNA, 54 nt, synthetic construct
  source 1..54: mol_type = other RNA
gtaccgtataaagtatgttgatgttccaataagcagatcatgttttttaagccg

SEQ ID NO: 9 - DNA, 36 bp, synthetic construct
  source 1..36: mol_type = other DNA
atatttcatacaactacatcgtctagtacaaaaaat

SEQ ID NO: 10 - DNA, 133 bp, synthetic construct
  source 1..133: mol_type = other DNA
ctcctccccttcctgcacccgtacccccgtggtctttgaataaagtctgagtgggcggcagcggccgggagaatcggagtgctagaggatgaccttaaatcagggacgatattataattgactaagatatctg

SEQ ID NO: 11 - DNA, 123 bp, synthetic construct
  source 1..123: mol_type = other DNA
aggaggggaaggacgtgggcatgggggcaccagaaacttatttcagactcacccgccgtcctcttagcctcacgatctcctactggaatttagtccctgctataatattaactgattctatag

(19) United States
(12) Patent Application Publication
(10) Pub. No.: US 2026/0201394 A1
(43) Pub. Date: Jul. 16, 2026

(54) METHODS OF MAKING RNA BY SPLINTED LIGATION AND COMPOSITIONS THEREOF

(71) Applicant: Orbital Therapeutics, Inc., Cambridge, MA (US)

(72) Inventors: Christopher Robert CARLSON, Cambridge, MA (US); David Eric WEINBERG, Cambridge, MA (US); Gilles BESIN, Cambridge, MA (US)

(21) Appl. No.: 19/630,613

(22) Filed: Mar. 27, 2026

Related U.S. Application Data
(63) Continuation of application No. PCT/US2024/048786, filed on Sep. 27, 2024.
(60) Provisional application No. 63/587,024, filed on Sep. 29, 2023.
(60) Provisional application No. 63/587,038, filed on Sep. 29, 2023.

(51) Int. Cl. C12N 15/66; C12N 9/00; C12N 9/12; C12N 9/16; C12N 15/10; C12N 15/11; C12P 19/34

(52) U.S. Cl. CPC C12N 15/66; C12N 9/1241; C12N 9/16; C12N 9/93; C12N 15/101; C12N 15/11; C12P 19/34; C12N 2310/333; C12N 2310/3341; C12N 2310/335; C12N 2840/203; C12Y 207/07019; C12Y 301/13001; C12Y 301/21001; C12Y 605/01003

(57) ABSTRACT

The present invention provides, among other things, a method of making circular RNA by annealing a DNA splint having regions of homology to the 5′ and 3′ ends of linear RNA with an RNA ligase and subsequently removing the DNA splint. Also provided is a method of joining two linear RNA molecules by ligation using a DNA splint having regions of homology to the 3′ end of a first linear RNA and the 5′ end of a second linear RNA with a RNA ligase, and subsequently removing the DNA splints. The present invention provides a method of making circular RNA by joining two linear RNA molecules with an RNA ligase and two DNA splints, each comprising regions of homology to the 5′ end.

### CROSS REFERENCE TO RELATED APPLICATIONS

This application is a Continuation Application of International Application No. PCT/US2024/048786, filed on Sep. 27, 2024, which claims priority to U.S. Provisional Application No. 63/587,038 filed on Sep. 29, 2023, and U.S. Provisional Application No. 63/587,024, filed on Sep. 29, 2023; the entirety of each of which is hereby incorporated by reference.

### INCORPORATION BY REFERENCE OF SEQUENCE LISTING

The present application is being filed along with a Sequence Listing submitted electronically in XML format. The Sequence Listing file “ORB-010WO1_SL” is created on Sep. 25, 2024, which is 11,775 bytes in size; the contents of which are incorporated herein for all purpose.

### BACKGROUND

Circular RNA is a highly stable closed loop structure that is resistant to degradation by exonucleases, making it attractive for generating stable RNA medicines, including RNA therapeutics and RNA vaccines.

Circular RNA is typically generated by chemical or enzymatic methods. Chemical ligation is effective for small circular RNAs from between 2-100 nucleotides, but efficiency is low due to intermolecular ligations and formation of 2′-5′ phosphodiester bonds, and it involves use of toxic reagents, which presents challenges for developing RNA medicines. Synthetic circular RNA can also be generated from their linear counterparts by enzymatic methods using RNA ligases and by intron mediated back-splicing.

In developing RNA therapeutics, one strategy for reducing RNA immunogenicity is to generate linear or circular RNA with 100% modified nucleotides, for example, 100% N1-methylpseudouridine (N1m-Ψ) or 100% 5-methoxyuridine (5mo-U). However, synthesis of circular RNA with 100% N1m-Ψ or 100% 5mo-U using current methods is either impossible or inefficient, leading to low RNA circularization and low yield. Thus, there is a need for new methods for generating RNA, including circular RNA, efficiently.

### SUMMARY OF THE INVENTION

The present invention provides, among other things, optimized splinted ligation methods for circularizing RNAs, including circularizing RNA comprising modified nucleotides. In addition, the present splinted ligation methods are suitable for annealing two or more RNA sequences to generate a RNA molecule in linear or circular format.

In some aspects, the present invention provides efficient methods of making circular RNA by bringing the 5′ and 3′ ends of a linear RNA in close proximity to each other using a splint fragment to form a single stranded region and ligating the singled stranded ends to form a circular RNA using RNA ligase. In particular, the splinted methods of the present invention can reduce intermolecular RNA ligation, thereby increasing intramolecular circularization to form circular RNA. Furthermore, the splinted methods include optimized purification procedures to purify circular RNA after ligation reaction, thereby, generating purified circular RNA. The present invention provides methods of making stable circular RNA medicines, including RNA therapeutics and RNA vaccines, with high stability, high translatability, and in some embodiments, comprise modified nucleotides leading to reduced immunogenicity.

In one aspect, the present invention provides a method for making a circular RNA by annealing a splint (e.g., a DNA splint) that comprises a gap and regions of complementarity to a 5′ and a 3′ end of a linear RNA, using an RNA ligase to ligate the single-stranded ends of the linear RNA molecule, thereby forming a circular RNA.

In some embodiments, the present invention provides DNA splints that carry out efficient circularization and ligation. Specifically, the inventors have ingeniously designed a DNA splint that has longer homology arms that comprise complementary sequences to the 5′ and 3′ ends of linear RNA molecule(s) to be ligated. Longer splint complementary sequences were unexpectedly found to provide greater circularization efficiency. Using this method, the inventors discovered that they were able to increase circularization efficiency to greater than 60% (e.g., 60%-70%).

In some embodiments, the DNA splint comprises a gap (e.g., 2-6 nucleotides, e.g., 4 nucleotides). By the present invention, the inventors found that the number of residues comprising the gap in concert with longer complementary sequences used in the DNA splint resolved previous challenges of low percentages of circularization using the splinted ligation method. The gap is comprised of any nucleotide (e.g., cytosine, guanine, adenine, thymine, including RNA nucleotides such as uridine), including homopolymeric stretch of nucleotides, modified nucleotides, or abasic sites. Low concentrations of RNA are used to prevent intermolecular ligation and a ratio of RNA:DNA splint (e.g., equimolar) provides high efficiency circularization.

In other embodiments, the splint comprises no gap, i.e., no nucleotides inserted between complementary sequence regions. The splint comprises only sequences complementary to the 5′ end and 3′ end of the linear RNA which makes single stranded overhangs on both 5′ and 3′ ends. The overhangs on each end may be 1-20 nucleotides in length.

Among other things, the present invention also provides a method of circularizing RNA that comprises modified nucleotides. In fact, RNA consisting of 100% modified nucleotides that cannot be circularized by other methods, can also be circularized by the present invention, thereby providing a method of generating stable RNA therapeutics with reduced immunogenicity. Without wishing to be bound by any particular theory, it is contemplated that similar to linear RNA, modified nucleotides in circular RNA such as pseudouridine, N1-methylpseudouridine (N1m-Ψ), and 5-methoxyuridine (5moU) evade cellular RNA sensors and subsequent activation of TLR and RIG-I pathways as in linear RNA (Kariko et al., 2005, Durbin et al., 2016). Some RNA modifications such as N6-methyladenosine (m6A), for example, promote cap-independent translation. Stable RNA therapeutics, including mRNA vaccines, with low immunogenicity are important for treating various diseases, such as cancer as well as infectious diseases. Circular RNA of the present invention, in some embodiments, comprising modified nucleotides, is administered as a pharmaceutical agent to treat disease.

In another aspect, the present invention provides circular RNA compositions generated by splinted ligation, which are comparable in translatability to circular RNA made by intron-mediated splicing. Further, circular RNA generated by a splinted ligation method of the present invention comprising up to about 20% modified nucleotides can also be translated. Stable circular RNA can be translated both in vitro and in vivo to produce polypeptides.

By the present invention, the splint is removed after ligation by various methods, e.g., denaturation by non-enzymatic or enzymatic methods. In some embodiments, enzymatic methods include use of a DNA nuclease, such as an exonuclease (e.g., DNase I). The splint may be biotinylated and removed by capture on streptavidin beads and purification. In some embodiments, the splint comprises abasic sites or uridine residues, which facilitate denaturation and removal of the splint. In some embodiments, the splint is removed using reverse complement splints, i.e., a reverse splint comprising nucleic acid sequences complementary to the splint. As a non-limiting example, high concentration of reverse splint (e.g., splint:reverse splint at a ratio of 1:10) are used to remove the splint in the ligation reaction. In some embodiments, the reverse splint further comprises a poly(A) sequence at the 5′ end or 3′ end. The poly(A) sequence can be used to remove the splint after ligation. In one embodiment, the poly(A) sequence comprises 5-200 nucleotides, e.g., 30 nucleotides.

In some embodiments, the present method further comprises removing un-ligated RNA from the ligation reaction. As a non-limiting example, the present splinted ligation method comprises a step of treating the ligation reaction with a poly A polymerase to add a poly(A) tail to any free 3′-OH on an RNA in the reaction. The un-ligated poly(A) tailed RNAs can be removed using an oligo (dT) column.

The inventors have provided a method herein that does not incorporate any residual extraneous DNA or RNA sequence from the ligation and circularization process, i.e., it is a scarless method. Further, the method limits the production of byproducts that need to be removed before use, streamlining the manufacturing process and reducing costs.

Circular RNA therapeutics manufactured by the method of the present invention can be administered in smaller doses with longer lasting RNA expression besides being less immunogenic, thereby minimizing side effects as well as leading to cost savings from manufacturing lower volumes.

In some aspects, the present disclosure provides methods of joining linear RNA molecules, for example that do not share homology with each other, through binding a DNA splint comprising a gap and regions of sequences complementary to the 3′ end and 5′ end of the two linear RNA molecules that generates a single stranded region, using a RNA ligase, and subsequently removing the splint and un-ligated RNAs. In some embodiments, the DNA splint provided by the present invention comprising a gap of specified number of residues and long complementary sequences increases ligation efficiency of two linear RNA molecules resulting in highly efficient scarless RNA ligation, without any byproducts that need to be removed. In some embodiments, the DNA splint comprises no gap between the longer complementary sequences in which the splint and RNAs hybrid generates short overhangs at the 5′ end and 3′ end, making a single stranded region for RNA ligase.

In some embodiments, two linear RNA molecules are joined by one DNA splint to form a linear RNA precursor, which is further circularized by another DNA splint of the present invention. In other embodiments, more than two RNA molecules can be joined by two or more DNA splints to form a linear RNA precursor, which is further circularized by another DNA splint of the present invention.

In some aspects, the present disclosure provides a method of making circular RNA, the method comprising: (i) contacting a linear RNA comprising a 5′ end and a 3′ end with a RNA ligase and a DNA splint comprising a sequence region complementary to the 5′ end of the linear RNA and a sequence region complementary to the 3′ end of the linear RNA, wherein the DNA splint binds the linear RNA and the ligase joins the 5′ end and the 3′ end; and (ii) removing the DNA splint; thereby generating circular RNA.

In some aspects, the present disclosure provides a method of joining two linear RNA molecules by binding a DNA splint, the method comprising: (i) contacting the two linear RNA molecules, each comprising a 5′ end and a 3′ end with a RNA ligase and a DNA splint comprising a first sequence region complementary to the 3′ end of the first linear RNA and a second sequence region complementary to the 5′ end of the second linear RNA, wherein the DNA splint binds the linear RNA and the ligase joins the 3′ end of the first linear RNA and the 5′ end of the second linear RNA; and (ii) removing the DNA splint; thereby generating a single linear RNA. Such single linear RNA can be used as mRNA therapeutics or as a linear RNA precursor to generate a circular RNA using ligation methods of the present invention.

In some embodiments, more than two linear RNA molecules are ligated to form a single RNA molecule with two or more DNA splints as described herein. As a non-limiting example, three linear RNA molecules can be ligated using 2 DNA splints as designed in the present invention.

In some embodiments, the splinted ligation method of the present invention further comprises a step of removing un-ligated RNAs; the method includes treating the reaction mixture with polyA polymerase to add a poly(A) tail to an free 3′OH on an RNA (un-ligated RNA) and removing poly(A)+ RNA from the reaction, thereby purifying circular RNA.

In some embodiments, the linear RNA and the DNA splint are contacted at a ratio of 4:1, 3:1, 2:1, 1:1, 1:0.5. In some embodiments, the linear RNA and the DNA splint are contacted at a ratio of 4:1. In some embodiments, the linear RNA and the DNA splint are contacted at a ratio of 3:1. In some embodiments, the linear RNA and the DNA splint are contacted at a ratio of 2:1. In some embodiments, the linear RNA and the DNA splint are contacted at a ratio of 1:1. In some embodiments, the linear RNA and the DNA splint are contacted at a ratio of 1:0.5.

In some embodiments, the linear RNA and the DNA splint are contacted in a 1:4, 1:3 or 1:2 ratio. In some embodiments, the linear RNA and the DNA splint are contacted at a ratio of 1:4. In some embodiments, the linear RNA and the DNA splint are contacted at a ratio of 1:3. In some embodiments, the linear RNA and the DNA splint are contacted at a ratio of 1:2. In some embodiments, the linear RNA and the DNA splint are contacted at a ratio of 1:1.

In some embodiments, the DNA splint is biotinylated.

In some embodiments, the DNA splint is single-stranded.

In some embodiments, the DNA splint comprises a gap. In some embodiments, the gap is single-stranded. In some embodiments, the single-stranded gap in the DNA splint is across from single-stranded RNA nucleotides.

In some embodiments, the gap is between 1-20 nucleotides in length. In some embodiments, the gap is between 2-6 nucleotides in length. In some embodiments, the gap is 2 nucleotides in length. In some embodiments, the gap is 3 nucleotides in length. In some embodiments, the gap is 4 nucleotides in length. In some embodiments, the gap is 5 nucleotides in length. In some embodiments, the gap is 6 nucleotides in length.

In some embodiments, the gap is between 7-20 nucleotides in length. In some embodiments, the gap is 7, 8, 9, 10, 11, 12, 13, 14 or 15 nucleotides in length. In some embodiments, the gap is 7 nucleotides in length. In some embodiments, the gap is 8 nucleotides in length. In some embodiments, the gap is 9 nucleotides in length. In some embodiments, the gap is 10 nucleotides in length. In some embodiments, the gap is 11 nucleotides in length. In some embodiments, the gap is 12 nucleotides in length. In some embodiments, the gap is 13 nucleotides in length. In some embodiments, the gap is 14 nucleotides in length. In some embodiments, the gap is 15 nucleotides in length. In some embodiments, the gap is 16 nucleotides in length. In some embodiments, the gap is 17 nucleotides in length. In some embodiments, the gap is 18 nucleotides in length. In some embodiments, the gap is 19 nucleotides in length. In some embodiments, the gap is 20 nucleotides in length.

In some embodiments, the gap is a single nucleotide.

In some embodiments, the gap is 4 nucleotides in length.

In some embodiments, the gap is comprised of adenine, thymine, uridine, guanine and/or cytosine nucleotides.

In some embodiments, the DNA splint comprises no gap. The DNA splint comprises sequences complementary to the 5′ end and 3′ end of an RNA, which generate overhangs (1-20 nucleotides) at the 5′ and/or 3′ ends.

In some embodiments, the gap is a homopolymeric stretch of nucleotides.

In some embodiments, the gap is comprised of adenine nucleotides. In some embodiments, the gap is comprised of thymine nucleotides. In some embodiments, the gap is comprised of guanine nucleotides. In some embodiments, the gap is comprised of cytosine nucleotides. In some embodiments, the gap is comprised of uridine nucleotides.

In some embodiments, the gap is comprised of one or more modified nucleotides and/or abasic sites. In some embodiments, the gap is comprised of modified nucleotides. In some embodiments, the modified nucleotide is pseudouridine, N1-methylpseudouridine (N1m-Ψ), and 5-methoxyuridine (5moU). In some embodiments, the modified nucleotide is pseudouridine. In some embodiments, the modified nucleotide is N1-methylpseudouridine (N1m-Ψ). In some embodiments, the modified nucleotide is 5-methoxyuridine (5moU). In some embodiments, the gap is comprised of one or more abasic sites. In some embodiments, the gap is comprised of one or more apurinic sites. In some embodiments, the gap is comprised of one or more apyrimidinic sites.

In some embodiments, the ligase is a RNA ligase.

In some embodiments, the ligase is T4 RNA ligase 1.

In some embodiments, the ligase is T4 RNA ligase 2.

In some embodiments, the ligase is a T4 RNA ligase 2, truncated KQ.

In some embodiments, the 5′ end is pre-adenylated.

In some embodiments, the region complementary to the 5′ end of the linear RNA and/or to the 3′ end of the linear RNA is at least 50 nucleotides long. In some embodiments, the complementary region is between about 50 to 500 nucleotides. In some embodiments, the complementary region is about 50 nucleotides. In some embodiments, complementary region is about 100 nucleotides. In some embodiments, the complementary region is about 150 nucleotides. In some embodiments, the complementary region is about 200 nucleotides. In some embodiments, the complementary region is about 250 nucleotides. In some embodiments, the complementary region is about 300 nucleotides. In some embodiments, the complementary region is about 350 nucleotides. In some embodiments, the complementary region is about 400 nucleotides. In some embodiments, the complementary region is about 450 nucleotides. In some embodiments, the complementary region is about 500 nucleotides. In some embodiments, the complementary region is greater than 500 nucleotides.

In some embodiments, the region complementary to the 5′ end of the linear RNA and/or to the 3′ end of the linear RNA is between 10 to 500 nucleotides long. In some embodiments, the region complementary to the 5′ end of the linear RNA and/or to the 3′ end of the linear RNA is between 20 to 120 nucleotides long.

In some embodiments, the region complementary to the 5′ end of the linear RNA and/or to the 3′ end of the linear RNA is 10 nucleotides long. In some embodiments, the region complementary to the 5′ end of the linear RNA and/or to the 3′ end of the linear RNA is 20 nucleotides long. In some embodiments, the region complementary to the 5′ end of the linear RNA and/or to the 3′ end of the linear RNA is 30 nucleotides long. In some embodiments, the region complementary to the 5′ end of the linear RNA and/or to the 3′ end of the linear RNA is 40 nucleotides long. In some embodiments, the region complementary to the 5′ end of the linear RNA and/or to the 3′ end of the linear RNA is 50 nucleotides long. In some embodiments, the region complementary to the 5′ end of the linear RNA and/or to the 3′ end of the linear RNA is 60 nucleotides long. In some embodiments, the region complementary to the 5′ end of the linear RNA and/or to the 3′ end of the linear RNA is 70 nucleotides long. In some embodiments, the region complementary to the 5′ end of the linear RNA and/or to the 3′ end of the linear RNA is 80 nucleotides long. In some embodiments, the region complementary to the 5′ end of the linear RNA and/or to the 3′ end of the linear RNA is 90 nucleotides long. In some embodiments, the region complementary to the 5′ end of the linear RNA and/or to the 3′ end of the linear RNA is 100 nucleotides long. In some embodiments, the region complementary to the 5′ end of the linear RNA and/or to the 3′ end of the linear RNA is 110 nucleotides long. In some embodiments, the region complementary to the 5′ end of the linear RNA and/or to the 3′ end of the linear RNA is 120 nucleotides long.

In some embodiments, the region complementary to the 5′ end of the linear RNA and/or to the 3′ end of the linear RNA is between 1% to 10% of the length of the linear RNA. In some embodiments, the region complementary to the 5′ end of the linear RNA and/or to the 3′ end of the linear RNA is between 1% to 10% of the length of the linear RNA. In some embodiments, the region complementary to the 5′ end of the linear RNA and/or to the 3′ end of the linear RNA is 1% of the length of the linear RNA. In some embodiments, the region complementary to the 5′ end of the linear RNA and/or to the 3′ end of the linear RNA is 2% of the length of the linear RNA. In some embodiments, the region complementary to the 5′ end of the linear RNA and/or to the 3′ end of the linear RNA is 3% of the length of the linear RNA. In some embodiments, the region complementary to the 5′ end of the linear RNA and/or to the 3′ end of the linear RNA is 4% of the length of the linear RNA. In some embodiments, the region complementary to the 5′ end of the linear RNA and/or to the 3′ end of the linear RNA is 5% of the length of the linear RNA. In some embodiments, the region complementary to the 5′ end of the linear RNA and/or to the 3′ end of the linear RNA is 6% of the length of the linear RNA. In some embodiments, the region complementary to the 5′ end of the linear RNA and/or to the 3′ end of the linear RNA is 7% of the length of the linear RNA. In some embodiments, the region complementary to the 5′ end of the linear RNA and/or to the 3′ end of the linear RNA is 8% of the length of the linear RNA. In some embodiments, the region complementary to the 5′ end of the linear RNA and/or to the 3′ end of the linear RNA is 9% of the length of the linear RNA. In some embodiments, the region complementary to the 5′ end of the linear RNA and/or to the 3′ end of the linear RNA is 10% of the length of the linear RNA. In some embodiments, the region complementary y to the 5′ end of the linear RNA and/or to the 3′ end of the linear RNA is 11% of the length of the linear RNA. In some embodiments, the region complementary to the 5′ end of the linear RNA and/or to the 3′ end of the linear RNA is 12% of the length of the linear RNA. In some embodiments, the region complementary to the 5′ end of the linear RNA and/or to the 3′ end of the linear RNA is 13% of the length of the linear RNA. In some embodiments, the region complementary to the 5′ end of the linear RNA and/or to the 3′ end of the linear RNA is 14% of the length of the linear RNA. In some embodiments, the region complementary to the 5′ end of the linear RNA and/or to the 3′ end of the linear RNA is 15% of the length of the linear RNA. In some embodiments, the region complementary to the 5′ end of the linear RNA and/or to the 3′ end of the linear RNA is 16% of the length of the linear RNA. In some embodiments, the region complementary to the 5′ end of the linear RNA and/or to the 3′ end of the linear RNA is 17% of the length of the linear RNA. In some embodiments, the region complementary to the 5′ end of the linear RNA and/or to the 3′ end of the linear RNA is 18% of the length of the linear RNA. In some embodiments, the region complementary to the 5′ end of the linear RNA and/or to the 3′ end of the linear RNA is 19% of the length of the linear RNA. In some embodiments, the region complementary to the 5′ end of the linear RNA and/or to the 3′ end of the linear RNA is 20% of the length of the linear RNA.

In some embodiments, the region complementary to the 5′ end of the linear RNA and/or to the 3′ end of the linear RNA has a 5′ overhang and/or a 3′ overhang.

In some embodiments, the 5′ overhang is between 1-20 nucleotides, or between 2-10 nucleotides in length. In some embodiments, the 5′ overhang is 2 nucleotides in length. In some embodiments, the 5′ overhang is 2 nucleotides in length. In some embodiments, the 5′ overhang is 3 nucleotides in length. In some embodiments, the 5′ overhang is 4 nucleotides in length. In some embodiments, the 5′ overhang is 5 nucleotides in length. In some embodiments, the 5′ overhang is 6 nucleotides in length. In some embodiments, the 5′ overhang is 7 nucleotides in length. In some embodiments, the 5′ overhang is 8 nucleotides in length. In some embodiments, the 5′ overhang is 9 nucleotides in length. In some embodiments, the 5′ overhang is 10 nucleotides in length. In some embodiments, the 5′ overhang is 15 nucleotides in length. In some embodiments, the 5′ overhang is 20 nucleotides in length.

In some embodiments, the 3′ overhang is between 2-10 nucleotides in length. In some embodiments, the 3′ overhang is 2 nucleotides in length. In some embodiments, the 3′ overhang is 3 nucleotides in length. In some embodiments, the 3′ overhang is 4 nucleotides in length. In some embodiments, the 3′ overhang is 5 nucleotides in length. In some embodiments, the 3′ overhang is 6 nucleotides in length. In some embodiments, the 3′ overhang is 7 nucleotides in length. In some embodiments, the 3′ overhang is 8 nucleotides in length. In some embodiments, the 3′ overhang is 9 nucleotides in length. In some embodiments, the 3′ overhang is 10 nucleotides in length. In some embodiments, the 3′ overhang is 15 nucleotides in length. In some embodiments, the 3′ overhang is 20 nucleotides in length.

In some embodiments, step (ii) comprises digesting with a DNA nuclease.

In some embodiments, the DNA nuclease is a DNA exonuclease.

In some embodiments, the DNA nuclease is DNase I.

In some embodiments, step (ii) comprises denaturing and binding to streptavidin.

In some embodiments, the linear RNA is synthetic RNA.

In some embodiments, the linear RNA is in vitro transcribed RNA.

In some embodiments, the in vitro transcription is in the presence of GMP.

In some embodiments, the in vitro transcribed RNA is treated with phosphatase.

In some embodiments, the in vitro transcribed RNA is treated with kinase.

In some embodiments, RppH converts triphosphate to monophosphate.

In some embodiments, the 5′ end is a 5′ monophosphate.

In some embodiments, the 3′ end is a 3′ hydroxyl.

In some embodiments, the linear RNA is messenger RNA.

In some embodiments, the linear RNA and/or circular RNA comprises one or more modified nucleotides.

In some embodiments, the modified nucleotide is one or more of N1-methylpseudouridine (N1m-ψ), 5-methoxyuridine (5moU), N6-methyladenosine (m6A), pseudouridine (ψ) or 5-methylcytosine (m5C).

In some embodiments, the modified nucleotide is N1-methylpseudouridine. In some embodiments, the modified nucleotide is one or more of 5-methoxyuridine. In some embodiments, the modified nucleotide is one or more of N6-methyladenosine. In some embodiments, the modified nucleotide is one or more of pseudouridine. In some embodiments, the modified nucleotide is one or more of 5-methylcytosine.

In some embodiments, the modified nucleotide is 100%. In some embodiments, the linear RNA and/or circular RNA comprises 100% modified nucleotides.

In some embodiments, the linear RNA and/or circular RNA comprises less than 50% modified nucleotides.

In some embodiments, the linear RNA and/or circular RNA comprises less than 20% modified nucleotides.

In some embodiments, the linear RNA and/or circular RNA comprises less than 10% modified nucleotides.

In some embodiments, an unmodified linear RNA and a modified linear RNA are ligated to form a linear RNA precursor which is circularized to generate a circular RNA. In some embodiments, the modified linear RNA comprises less than 10%, or less than 20%, or less than 50% modified nucleotides.

In some embodiments, an unmodified linear internal ribosome entry site (IRES) element and a modified linear RNA coding a protein of interest are ligated to form a circular RNA for expressing the protein of interest. In some embodiments, the unmodified IRES element lacks chemical modifications. For example, the IRES elements lacks N1-methyl-pseudouridine (N1m-ψ) (e.g., less than 10% N1m-ψ). In some embodiments, the modified coding sequence comprises at least one modification that increases the stability and/or translation of the circular RNA, and/or decreases immunogenicity of the circular RNA.

In some embodiments, two modified linear RNAs are ligated to form a linear RNA precursor which is circularized to generate a circular RNA. As a non-limiting example, one modified RNA is 100% modified (i.e., all nucleotides are modified nucleotides) and the other comprises less than 10%, or less than 20%, or less than 50% modified nucleotides.

In some embodiments, two unmodified linear RNAs are ligated to form a linear RNA precursor which is circularized to generate a circular RNA. For example, an unmodified linear IRES element and an unmodified linear RNA coding a protein of interest are ligated to form a linear RNA precursor which is circularized to generate a circular RNA for expressing the protein of interest.

In some embodiments, the linear RNA and/or circular RNA does not comprise an intron.

In some embodiments, the linear RNA and/or circular RNA does not comprise homology arms.

In some embodiments, the linear RNA and/or circular RNA is between 100 bp to 10 kb in length.

In some embodiments, the linear RNA and/or circular RNA is between 100 bp to 500 bp in length.

In some embodiments, the linear RNA and/or circular RNA is between 500 bp to 10 kb in length.

In some embodiments, the linear RNA and/or circular RNA encodes a therapeutic protein. In some embodiments, the linear RNA and/or circular RNA encodes an immunogen.

In some embodiments, the linear RNA and/or circular RNA is a RNA vaccine.

In some embodiments, provided herein is a circular RNA generated by the methods described herein.

In some embodiments, the circular RNA comprises one or more modified nucleotides, wherein the modified nucleotide is N1-methylpseudouridine or 5-methoxyuridine.

In some embodiments, the modified nucleotide is N1-methylpseudouridine.

In some embodiments, the modified nucleotide is 5-methoxyuridine.

In some embodiments, the circular RNA comprises one or more modified nucleotides, wherein the modified nucleotide is 100%.

In some embodiments, the circular RNA comprises one or more modified nucleotides, wherein the modified nucleotide is less than 50%.

In some embodiments, the circular RNA comprises one or more modified nucleotides, wherein the modified nucleotide is less than 20%.

In some embodiments, the circular RNA comprises one or more modified nucleotides, wherein the modified nucleotide is less than 10%.

In some embodiments, provided herein is a method of manufacturing RNA comprising a step of circularizing RNA.

In some aspects, provided herein is a method of making circular RNA, the method comprising: contacting two linear RNA molecules, each comprising a 5′ end and a 3′ end with a RNA ligase, and two DNA splints, wherein the first DNA splint comprises two regions complementary to the 3′ end of the first linear RNA and to the 5′ end of the second linear RNA, wherein the second DNA splint comprises two regions complementary to the 3′ end of the second linear RNA and to the 5′ end of the first linear RNA, such that the two DNA splints bind the linear RNA molecules and the ligase joins the ends of the linear RNA molecules; and (ii) removing the DNA splints; thereby generating a circular RNA. In some embodiments, the first and second linear RNAs are first annealed with the first DNA splint to form a third linear RNA molecule and wherein the third RNA molecule is circularized with the second DNA splint, thereby generating the circular RNA.

In some embodiments, the method further comprising ligating two or more fragments to form a linear RNA precursor for circularization.

In some embodiments, the two linear RNAs and two DNA splints are mixed in one solution, thereby generating the circular RNA. In other embodiments, the first linear RNA is selected from an Internal Ribosome Entry Site (IRES) element, a microRNA binding site, a microRNA, a miRNA sponge, an inhibitory RNA sequence, and an active RNA sequence.

In some embodiments, the at least two linear RNA molecules comprise an Internal Ribosome Entry Site (IRES) element, a coding polynucleotide that encodes a polypeptide of interest, a microRNA binding site, a microRNA, an inhibitory sequence, an active sequence, a 3′ untranslated region (3′ UTR), a 5′ untranslated region (5′ UTR), an aptamer, and/or a signal sequence. In some embodiments, the second linear RNA is a coding sequence that encodes a polypeptide of interest. As a non-limiting example, one linear RNA is an Internal Ribosome Entry Site (IRES) element, and a second linear RNA is a messenger RNA that encodes protein.

In some embodiments, the first linear RNA is not modified and the second RNA fragment is chemically modified. In some embodiments, both the first and second RNA fragments are modified. In some embodiments, both the first and second RNA fragments are unmodified. In some embodiments, the first linear RNA is chemically modified and the second RNA fragment is unmodified.

In some embodiments, the method further comprises concentrating the reaction of linear RNA and DNA splint binding before addition of ligase to increase ligation efficiency.

In some embodiments, the method further comprises purifying the circular RNA using HPLC and/or SEC (size exclusion chromatography). For example, the circular RNA is purified using Ion-paired Reverse Phase HPLC.

In some embodiments, one linear RNA is an Internal Ribosome Entry Site (IRES) element, and a second linear RNA is a messenger RNA that encodes protein.

Any numerals used in this application with or without the terms “about” or “approximately” are meant to cover any normal fluctuations appreciated by one of ordinary skill in the relevant art.

Other features, objects, and advantages of the present disclosure are apparent in the detailed description that follows. It should be understood, however, that the detailed description, while indicating embodiments of the present disclosure, is given by way of illustration only, not limitation. Various changes and modifications within the scope of the disclosure will become apparent to those skilled in the art from the detailed description.

### BRIEF DESCRIPTION OF THE DRAWINGS

Drawings are for illustration purposes only; not for limitation.

FIG. 1A is a schematic of a method of making circular RNA by splinted ligation. Linear RNA was annealed with a DNA gap splint comprising regions of homology to the 5′ and 3′ ends of linear RNA. The splint secured the two ends of linear RNA in place to be ligated by a ssRNA ligase, for example, T4 RNA ligase I. Following subsequent DNase treatment to remove the splint, circular RNA was generated. FIG. 1B is a schematic of a method of making circular RNA using a DNA splint binding to the 5′ and 3′ ends of an RNA molecule without a gap.

FIG. 2A is a diagram showing splints with different homology length and gap size. 6 DNA splints were designed and tested. Splint #1 has a 28 nucleotide homology arm with a 4 nucleotide gap; Splint #2 has a 28 nucleotide homology arm with a 6 nucleotide gap; Splint #3 has a 63 nucleotides homology arm with a 4 nucleotide gap; Splint #4 has a 63 nucleotides homology arm with a 2 nucleotide gap; Splint #5 has a 63 nucleotides homology arm with a 0 nucleotide gap; Splint #6 has a 98 nucleotide homology arm with a 4 nucleotide gap.

FIG. 2B is a representative gel image that shows that splint #1 (28 nucleotide homology arm, 4 nucleotide gap) and splint #2 (28 nucleotide homology arm, 6 nucleotide gap) did not promote circularization to an appreciable extent. The linear band was dominant. The ligation was performed at 25° C. for 2 hours with a RNA concentration of 0.5 μM. Various concentrations of splint #1 and #2 were tested. The gel shows that splint #1 at 1 μM had maximum circularization, but barely above background. Ligation was slightly increased with splint #1 comprising a 4 nucleotide gap relative to splint #2 comprising a 6 nucleotide gap.

FIG. 2C is a representative gel image that shows that a splint #3 with longer homology arms (63 nucleotides) circularized more efficiently than splint #1 (28 nucleotide long homology arms), as seen from the ratio of the circular RNA band to linear unligated band. Both splints have a 4 nucleotide gap. Both splints circularized RNA efficiently at low concentrations of 0.25 μM. The addition of 10% polyethylene glycol (PEG) made no appreciable difference to circularization efficiency.

FIG. 2D is a representative gel image that shows that greater circularization was achieved by using a DNA splint with longer homology arms. Splint #3 showed more effective circularization than splint #4 and splint #5 which have smaller gap, indicating that the 4 nucleotide gap was effective. Splint #6 has longer homology arms, which promoted more circularization. The RNA concentration was lowered to 50 nM to reduce intermolecular ligation and show more faint upper bands on the gel that denote linear ligation instead of circularization.

FIG. 2E is a representative gel image that shows maximum circularization occurred when a DNA splint (#3) was approximately the same concentration as RNA, i.e., at equimolar ratios.

FIG. 3 shows a t-RNA like splint, splint #7, which includes 63 nucleotide long homology arms to the 5′ and 3′ ends of RNA, a 2 nucleotide overhang at the 5′ end and a 5 nucleotide overhang at the 3′ end but has no gap between two complementary sequence regions. Splint #3 is also shown for comparison.

FIG. 4 is a representative gel image that shows ligase reactions at 25° C. or 16° C. for 2 hours or 18 hours. Circularization dependence of enzymatic 5′ monophosphate generation (Dephos+phos) vs. excess GMP used in IVT reaction was tested. As shown in this figure, Splint #3 shows greater circularization than splint #7. The optimal ligation temperature and time is 2 hours at 25° C., or 18 hrs at 16° C. Further, the results showed that 5′ monophosphate end in linear RNA, necessary for ligation, generated by dephosphorylation of 5′ GTP with Antarctic phosphatase followed by subsequent phosphorylation with polynucleotide kinase treatment resulted in the same level of ligation as in vitro transcribing RNA with 5 fold excess of GMP to GTP.

FIG. 5 a representative gel image that shows RNA modified with 100% 5-methoxyuridine (5moU) and N1-methylpseudouridine (N1m-ψ) can be circularized by a DNA gap splint and ligated by a ssRNA ligase, T4 RNA ligase I.

FIG. 6 is a representative histogram showing translation of 100% N1m-ψ or 5mo-U modified circular RNAs. Circular RNA generated by splinted ligation is translatable, when unmodified. In contrast, modified circular RNAs, comprising exemplary modifications such as uridine, N1-methylpseudouridine (N1m-Ψ) or 5-methoxyuridine (5moU), circularized by the DNA splint method are not translated.

FIG. 7 shows the translation efficiency of circularized RNA. The graph shows that circRNAs generated by ligation (bar 2, 0% modified) are comparable in translatability relative to circRNAs made by intron-mediated splicing (bar 1, intron). Further, the graph compares translation of modified circular RNAs generated by ligation and shows that circular RNAs with 5% N1-methylpseudouridine (N1m-Ψ) were translated with a similar efficiency to unmodified RNAs, circular RNAs with 20% N1-methylpseudouridine (N1m-Ψ) were translated with about 50% the efficiency of unmodified RNAs, while circular RNAs with greater than 20% N1-methylpseudouridine (N1m-Ψ) were not translated efficiently in cells.

FIG. 8 is a schematic diagram illustrating two-step/multiple-step ligation process.

FIG. 9A illustrates two separate ligation steps to join two RNA fragments to form a chimeric circular RNA. FIG. 9B is a representative gel image showing circularization of a RNA using two-step ligation method illustrated in FIG. 9A. FIG. 9C is a gel image showing circular RNA after HPLC purification.

FIG. 10A illustrates one step ligation to form a chimeric circular RNA using two or more DNA splints in one reaction. FIG. 10B is a denaturing gel image of ligated RNA products: (production scheme outlined on the left, IRES transcribed with 100% UTP and coding sequence transcribed with 100% N1m-UTP).

FIG. 11A is a schematic showing concentrating the annealing reaction of linear RNA and DNA splint and ligation of the concentrated sample. FIG. 11B is a representative denaturing agarose gel depicting increased ligation efficiency (more circular RNA) when reaction is concentrated after splint annealing but before ligation. The reaction was 10× more concentrated. 0.1×, 0.2×, 0.4× (i.e., the amount of total ligase used relative to the standard method without concentration) ligase was used.

FIG. 12 is a representative gel image that shows un-ligated RNA in a ligation reaction is efficiently polyadenylated by polyA polymerase.

FIG. 13 is a graph showing expression levels of circular RNAs circularized using splint ligation which include unmodified IRES or N1m-ψ modified IRES. The circular RNAs with an unmodified IRES were translated to produce the reporter protein, while the circular RNA including a N1m-ψ modified IRES was not effectively translated.

FIG. 14A-FIG. 14B shows cytokine response and abundance of circular RNAs circularized using splint ligation method in mice. The circular RNAs are unmodified or 100% N1m-ψ modified. FIG. 14A shows cytokine responses to unmodified and 100% N1m-ψ modified circular RNAs in mice 6 hours post-injection. FIG. 14B shows abundance of circular RNAs in mice 6 hours post-injection.

### DEFINITIONS

In order for the present disclosure to be more readily understood, certain terms are first defined below. Additional definitions for the following terms and other terms are set forth throughout the specification.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). In some embodiments, the term refers to a range of values that fall within 10% of the stated reference value. In some embodiments, the term refers to a range of values that fall within 5% of the stated refer. The term “between” includes the values of the specified boundaries and all intervening values and fractions.

Abasic site: As used herein, an “abasic site” is a location in RNA or DNA that has neither a purine nor a pyrimidine base (apurinic or apyrimidinic site or AP site). AP sites result from cleavage of the N-glycosylic bond between the nitrogenous base and the deoxyribose sugar, leaving an intact phosphodiester backbone. AP sites are frequently generated spontaneously or due to DNA damage.

Adenylation: As used herein, “adenylation” or “AMPylation” is a process in which an adenosine monophosphate (AMP) molecule is covalently attached to the amino acid side chain of a protein. This covalent addition of AMP to a hydroxyl side chain of the protein is a posttranslational modification.

Antibody: As used herein, the term “antibody” is referred to in the broadest sense and specifically covers various embodiments including, but not limited to monoclonal antibodies, polyclonal antibodies, multispecific antibodies (e.g., bispecific antibodies formed from at least two intact antibodies), and antibody fragments (e.g., diabodies) so long as they exhibit a desired biological activity (e.g., “functional”). Antibodies are primarily amino-acid based molecules but may also comprise one or more modifications (including, but not limited to the addition of sugar moieties, fluorescent moieties, chemical tags, etc.). Non-limiting examples of antibodies or fragments thereof include VH and VL domains, scFvs, Fab, Fab′, F(ab′)2, Fv fragment, diabodies, linear antibodies, single chain antibody molecules, multispecific antibodies, bispecific antibodies, intrabodies, monoclonal antibodies, polyclonal antibodies, humanized antibodies, codon-optimized antibodies, tandem scFv antibodies, bispecific T-cell engagers, mAb2 antibodies, chimeric antigen receptors (CAR), tetravalent bispecific antibodies, biosynthetic antibodies, native antibodies, miniaturized antibodies, unibodies, maxibodies, antibodies to senescent cells, antibodies to conformers, antibodies to disease specific epitopes, or antibodies to innate defense molecules.

Antibody fragment: As used herein, “antibody fragment” refers to any portion of an intact antibody. In some embodiments, antibody fragments comprise antigen binding regions from intact antibodies. Examples of antibody fragments may include, but are not limited to Fab, Fab′, F(ab′)2, and Fv fragments; diabodies; linear antibodies; single-chain antibody molecules; and multispecific antibodies formed from antibody fragments.

Antibody variant: As used herein, “antibody variant” refers to a biomolecule resembling an antibody in structure and/or function comprising some differences in their amino acid sequence, composition or structure as compared to a native antibody.

Biologic: As used herein, a “biologic” is a polypeptide-based molecule encoded by the linear and/or circular RNA provided herein which may be used to treat, cure, mitigate, prevent, or diagnose a disease or disorder.

Circular RNA: As used herein, “circular RNA” or “circRNA” refer to an RNA that forms a circular structure through covalent or non-covalent bonds. A circular RNA can be endogenous or synthetic. Circular RNAs endogenous to eukaryotic cells comprise a large class of non-coding RNAs that are produced by a non-canonical splicing event called back-splicing. Endogenous circRNAs are covalently closed, single stranded RNA molecules formed by the back-splicing of linear precursor mRNA or lncRNA transcripts. Endogenous circular RNAs (circRNAs) exert important biological functions by acting as microRNA or protein inhibitors (‘sponges’), by regulating protein function or by encoding polypeptides. CircRNAs have been implicated in diseases such as diabetes mellitus, neurological disorders, cardiovascular diseases, cancer, etc. Unlike linear mRNAs, circular RNAs are resistant to exonucleolytic decay and have significantly improved drug like properties compared to mRNA therapeutics including enhanced longevity as protein production vectors.

Complementary Region: As used herein, ‘complementary region refers to flanking sequences, one at either end of a DNA splint. A DNA splint recognizes and binds to the 5’ end and 3′ end of RNA molecules, based on complementary regions e.g., full or partial complementarity to the 5′ end and 3′ end of one or more RNA molecules, and brings the 5′ and 3′ ends together. In some embodiments, each complementary region is between 10 nucleotides to 500 nucleotides long. In some embodiments, each complementary region is at least 50 nucleotides long. In some embodiments, each complementary is between 50 to 120 nucleotides long.

Delivery: As used herein, “delivery” refers to the act or manner of delivering a compound, substance, entity, moiety, cargo or payload.

DNA Nuclease: As used herein, “DNA nuclease” is an enzyme that catalyzes the cleavage of phosphodiester bonds. DNA nucleases play a role in DNA replication and various DNA repair processes, base excision repair, nucleotide excision repair, mismatch repair, and double strand break repair. Depending on whether a 5′ or 3′ end is required for substrate recognition and whether cleavage products are single or oligo nucleotides, DNA nucleases are classified as exonucleases and endonucleases. For example, self-cleaving ribozymes cleave RNAs endonucleolytically while exonucleases cleave one nucleotide at a time from one end, either from 5′ to 3′ or from 3′ to 5′. Some nucleases have both exonuclease and endonuclease activities, for example, Flap endonuclease 1 (FEN1) has 5′ to 3′ exonuclease activity in addition to endonuclease activity and Mre11, has both endo and 3′ to 5′ exonuclease activities.

DNase I: As used herein, “DNase I” or “deoxyribonuclease I” refers to enzymes that cleave single or double-stranded DNA and require divalent metal ions to hydrolyze DNA yielding 3′-hydroxyl and 5′-phosphorylated products.

Encode: As used herein, the term “encode” or “encoding” refers broadly to any process whereby the information in a polymeric macromolecule is used to direct the production of a second molecule that is different from the first. The second molecule may have a chemical structure that is different from the chemical nature of the first molecule. For example, DNA encodes RNA and RNA encodes a polypeptide or protein.

Enhance: As used herein, the terms “enhance” and “enhancement” refers to an increase of at least about 5%, 10%, 20%, 25%, 50%, 75%, 100%, 150%, 200%, 300%, 400%, 500% or more of a reference; the reference may be a biological function of a nucleic acid or protein and a gene expression level, etc.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing) (RNA expression); (3) translation of an RNA into a polypeptide or protein; and (4) post-translational modification of a polypeptide or protein (Protein expression).

Feature: As used herein, a “feature” refers to a characteristic, a property, or a distinctive element. when referring to polypeptides are defined as distinct amino acid sequence-based components of a molecule. Features of the polypeptides encoded by the present circular polynucleotide, such as surface manifestations, local conformational shape, folds, loops, half-loops, domains, half-domains, sites, termini or any combination thereof.

Formulation: As used herein, a “formulation” includes at least one compound, substance, entity, moiety, cargo or payload and a delivery agent.

Gap: As used therein, the term “gap” refers to a small region with the DNA splint comprising a stretch of nucleotides that are not complementary to any sequence of linear RNAs that are to be circularized. The gap region can be in the middle nucleotides of the splint, creating a situation where the splint ‘gap’ is single stranded, and the terminal 3′ and 5′ ends of the RNA molecule are also single stranded (depending on size of the gap).

Homology: As used herein, the term “homology” refers to the overall relatedness between polymeric molecules, e.g., between polynucleotide molecules (e.g., DNA molecules and/or RNA molecules). In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two polynucleotide sequences. In accordance with the disclosure, two polynucleotide sequences are considered to be homologous if the polypeptides they encode are at least about 50%, 60%, 70%, 80%, 90%, 95%, or even 99% identical for at least one stretch of at least about 20 amino acids. In some embodiments, homologous polynucleotide sequences are characterized by the ability to encode a stretch of at least 4-5 uniquely specified amino acids. For example, in some embodiments, for polynucleotide sequences less than 60 nucleotides in length, homology is determined by the ability to encode a stretch of at least 4-5 uniquely specified amino acids.

Identity: As used herein, the term “identity” refers to the overall relatedness between polymeric molecules, e.g., between polynucleotide molecules (e.g., DNA molecules and/or RNA molecules) and/or between polypeptide molecules. Calculation of the percent identity of two polynucleotide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, considering the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H. and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12 (1), 387 (1984)), BLASTP, BLASTN, and FASTA Altschul, S. F. et al., J. Molec. Biol., 215, 403 (1990)).

Homopolymeric: As used herein, “homopolymer” or “homopolymeric nucleotides,” and grammatical equivalents thereof, refers to a sequence of consecutive identical bases. In some embodiments, the nucleotides comprising the homopolymeric nucleotides are selected from A, T, G, C or U. In some embodiments, the nucleotides are modified nucleotides. In some embodiments, the term “homopolymer” or “homopolymeric nucleotides,” refers to a sequence of substantially identical bases. For example, in some embodiments, the term includes a consequence series of nucleotides that has one or more non-identical nucleotides.

Intron: As used herein, the term “intron” means any sequence within a gene that is removed by RNA splicing during maturation of RNA transcripts from pre-mRNA transcripts. Introns in general are non-coding sequences of an RNA transcript.

Lipid Nanoparticle: As used herein “lipid nanoparticle” or “LNP” refers to a delivery vehicle comprising one or more lipids (e.g., cationic lipids, non-cationic lipids, PEG-modified lipids). A “ionizable lipid” refers to any of a number of lipid species that carry a net positive charge at a selected pH. A “non-cationic lipid” refers to any neutral, zwitterionic or anionic lipid.

Liposome: As used herein “liposome” generally refers to a vesicle composed of lipids (e.g., amphiphilic lipids) arranged in one or more spherical bilayers or bilayers.

Modified: As used herein “modified” or, as appropriate “modification” refers to a changed state or structure of a molecule. Molecules may be modified in many ways including chemically, structurally, and functionally. With respect to nucleic acid molecules (e.g., DNA and RNA), the modifications are modified version of A, G, C, U or T nucleotides. Generally, herein, these terms are not intended to refer to the ribonucleotide modifications in naturally occurring 5′-terminal mRNA cap moieties. With respect to polypeptides, the term “modification” refers to a modification as compared to the canonical set of 20 amino acids.

mRNA: As used herein, the term “messenger RNA” (mRNA) means a polynucleotide which encodes a polypeptide of interest and which is capable of being translated to produce the encoded polypeptide of interest in vitro, in vivo, in situ or ex vivo.

miRNA sponge: As used herein, the term “miRNA sponge” refers to a circular polynucleotide comprising a single-stranded non-coding polynucleotide with repeat copies of at least one specific microRNA binding site to hold microRNA molecules of interest. The miRNA sponge acts as an artificial microRNA inhibitor, when expressed in a cell, would decrease the cellular level of the microRNA of interest.

miRNA Response Element (MRE): As used herein, the term “miRNA response element (MRE)” refers to a target site (i.e., a short nucleic acid fragment) that binds to a miRNA. In some embodiments, the circular polynucleotide comprise two or more MREs. The number of MREs in the circular polynucleotide is variable and relates to the length of the circular polynucleotide. As non-limiting examples, the circular polynucleotide comprises 2, 3, 4, 5, 6, 7, 8, 9, 10 or more MREs. The multiple MREs may have the same nucleic acid sequences and bind to the same miRNA; or alternatively, the MREs have different nucleic acid sequences and bind to different miRNAs, such as 2, 3, 4, 5, or more different miRNAs.

Monoclonal Antibody: As used herein, the term “monoclonal antibody” refers to an antibody obtained from a population of substantially homogeneous cells (or clones), i.e., the individual antibodies comprising the population are identical and/or bind the same epitope, except for possible variants that may arise during production of the monoclonal antibodies, such variants generally being present in minor amounts. In contrast to polyclonal antibody preparations that typically include different antibodies directed against different determinants (epitopes), each monoclonal antibody is directed against a single determinant on the antigen. The modifier “monoclonal” indicates the character of the antibody as being obtained from a substantially homogeneous population of antibodies and is not to be construed as requiring production of the antibody by any particular method. The monoclonal antibodies herein include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies.

Nucleoside: As described herein, “nucleoside” is defined as a compound containing a sugar molecule (e.g., a pentose or ribose) or a derivative thereof in combination with an organic base (e.g., a purine or a pyrimidine) or a derivative thereof (also referred to herein as “nucleobase”). Five primary/canonical nucleobases: adenine (A), cytosine (C), guanine (G), thymine (T), and uracil (U) are the fundamental units of nucleic acid molecules, in which Adenine and guanine, referred to purine bases, have a fused-ring skeletal structure derived of purine while uracil, and thymine, derived of pyrimidine, are referred to pyrimidine bases.

Nucleotide: As described herein, “nucleotide” is defined as a nucleoside including a phosphate group or other backbone linkage (internucleoside linkage).

Preadenylation: As used herein, “preadenylation” or “preadenylated” refers to adenylation prior to ligation. In some embodiments, the 5′ end is preadenylated. In some embodiments, 5′ adenylated RNA (5′ AppRNA) removes the need to dephosphorylate RNA prior to ligation and prevents undesirable ligation products. In some embodiments, a truncated KQ ligase ligates 3′ end of ssRNA to 5′ adenylated ssDNA or 5′ adenylated ssRNA.

Pharmaceutical Composition: As used herein the term “pharmaceutical composition” refers to compositions comprising at least one active ingredient and optionally one or more pharmaceutically acceptable excipients.

Polypeptides of interest: As used herein, the term “polypeptides of interest” refer to any polypeptide encoded in the linear and/or circular RNA of the present invention. As used herein, “polypeptide” means a polymer of amino acid residues (natural or unnatural) linked together, most often by peptide bonds. In some embodiments, the term, as used herein, refers to proteins (i.e., proteins of interest), polypeptides, and peptides of any size, structure, or function. In some embodiments, the polypeptide encoded is smaller than about 50 amino acids and the polypeptide is then termed a peptide. For example, a peptide is at least about 2, 3, 4, or at least 5 amino acid residues long. In some embodiments, polypeptides include gene products, naturally occurring polypeptides, synthetic polypeptides, homologs, orthologs, paralogs, fragments and other equivalents, variants, and analogs of the foregoing. In some embodiments, a polypeptide is a single molecule. In some embodiments, a polypeptide includes a multi-molecular complex such as a dimer, trimer or tetramer. In some embodiments, a polypeptide comprises single chain or multichain polypeptides such as antibodies or insulin and is associated or linked. In some embodiments, disulfide linkages are found in multichain polypeptides. In some embodiments, the term polypeptide applies to amino acid polymers in which one or more amino acid residues are an artificial chemical analogue of a corresponding naturally occurring amino acid.

Polypeptide variant: As used herein, “polypeptide variant” refers to molecules which differ in their amino acid sequence from a native, wild-type or reference sequence. In some embodiments, the amino acid sequence variants possess substitutions, deletions, and/or insertions at certain positions within the amino acid sequence, as compared to a native or reference sequence. Typically, variants possess at least about 50% identity (homology) to a native or reference sequence. In some embodiments, they possess at least about 80%, at least about 90% identity to a native, wild-type or reference sequence.

Signal peptide: As used herein, a “signal sequence” or “signal peptide” is a polynucleotide or polypeptide, respectively, which is from about 9 to 200 nucleotides (3-60 amino acids) in length which is incorporated at the 5′ terminus of the coding region or the N-terminus polypeptide encoded, respectively. In some embodiments, addition of these sequences result in trafficking of the encoded polypeptide to the endoplasmic reticulum through one or more secretory pathways. Some signal peptides are cleaved from the protein by signal peptidase after the proteins are transported.

Subject: As used herein, the terms “subject” and “patient” may be used interchangeably herein. As such, a “subject” includes a human that is being treated for a disease, or prevention of a disease, such as a patient.

Transcription: As used herein the term “transcription” refers to the formation or synthesis of an RNA molecule by an RNA polymerase using a DNA molecule as a template.

Translation: As used herein the term “translation” refers to the formation of a polypeptide molecule by a ribosome based upon an RNA template.

Treat and Prevent: As used herein the terms “treat” or “prevent” as well as words stemming therefrom do not necessarily imply 100% or complete treatment or prevention. Rather there are varying degrees of treatment or prevention of which one of ordinary skill in the art recognizes as having a potential benefit or therapeutic effect. Also, “prevention” can encompass delaying the onset of the disease, symptom or condition thereof.

Unmodified: As used herein, “unmodified” refers to any substance, compound or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

Therapeutically effective amount: As used herein, the term “therapeutically effective amount” means an amount effective, at dosages, frequency of administration and for duration of time necessary to achieve the desired results such that one or more symptoms or biomarkers is improved after treatment.

### DETAILED DESCRIPTION

The present invention provides, among other things, efficient methods of making circular RNA by annealing a DNA splint that comprises a gap of specified nucleotides (e.g., 2-6 nucleotides), including modified nucleotides, or abasic sites, and long regions of homology (e.g., 10-500 nucleotides) with a 5′ and a 3′ end of a linear RNA, using a RNA ligase, and subsequently removing the splint, providing methods of making stable RNA therapeutics, including mRNA vaccines, that are highly stable and translatable, and in some embodiments, comprise modified nucleotides leading to favorable properties such as low immunogenicity.

The DNA splint of the present invention has long complementary regions (e.g., between 10 to 500 nucleotides) that are complementary to the 5′ and 3′ ends of linear RNA molecule(s) to be ligated, and were unexpectedly found to provide efficient circularization with greater than 60% efficiency (e.g., 60%-75%). The DNA splint also comprises a gap (e.g., 1-20 nucleotides, 2-6 nucleotides, e.g., 4 nucleotides), which together with the long homology arms provided high efficiency circularization. Alternatively, the DNA splint may comprise no gap.

RNA consisting of 100% modified nucleotides (e.g., pseudouridine, N1-methylpseudouridine (N1m-Ψ), and 5-methoxyuridine (5moU)) that cannot be circularized by other methods, can also be circularized by the present invention, thereby providing a method of generating stable RNA therapeutics with low immunogenicity that are important for treating diseases such as cancer and for use in mRNA vaccines against infectious diseases.

### Circular RNA

Circular RNAs (circRNAs) are closed loop single-stranded RNAs with a contiguous structure that has enhanced stability and a lack of 5′ and 3′ end motifs. Unlike linear RNAs which interact with various cellular proteins through their 5′ and 3′ ends, circular RNAs lack this capacity and are more stable and conserved than linear RNA. Circular RNAs have a variety of protein-coding and non-coding functions, for example, among other things, they function as miRNA sponges to regulate miRNA expression and mRNA expression, transcriptional regulation by binding RNA binding proteins, protein translocation between cytosol and nucleus, protein-protein interactions, and enhancing protein translation. Circular RNAs play diverse important roles that circular RNAs play, the present invention provides a method of making circular RNA for use in therapeutics by targeting or exploiting one or more circRNA functions.

In some embodiments, circRNAs contain miRNA binding sites; they can function as miRNA sponges to regulate miRNA expression, eliminating miRNA-mediated repression of linear RNA transcripts and promoting mRNA expression. For example, circ-ITCH binds to miR-17 and miR-224 to act as a miRNA sponge, inhibiting bladder cancer through regulation of p21 and PTEN 31. CircCCDC9 binds to miR-6792-3p inhibiting the development of gastric cancer through regulation of CAV1. Circular RNAs can regulate transcription by binding RBPs. For example, circRNA formed by the insulin gene binds an RNA binding protein, TDP-43 and regulates insulin secretion. CircPABPN1 binds an RNA binding protein, HuR, thereby reducing binding of HuR to PABPN1 mRNA, and inhibiting translation of PABPN1. Circular RNAs also can form three dimensional structures by interacting with other proteins and regulate signaling. For example, circFoxo3, p53 and MDM2 forms a complex and promotes p53 ubiquitination and degradation. In addition to regulatory functions, circular RNAs function in translocating proteins, for example, circFoxo3 can bind multiple proteins such as ID-1, E2F1, HIF1a and FAK and promote their retention in cytoplasm. In contrast, circAmotl1 binds c-myc and facilitates its translocation to the nucleus, improving stability and target binding. CircAmotl1 binds and promotes STAT3 translocation to the nucleus. Importantly, same as their linear mRNA counterparts, circular RNAs comprising, for example, m6A modifications or IRES can be translated. CircRNAs containing multiple ORFs without stop codons can be translated by rolling circle translation. For example, circMb13 and circ-ZNF609 can be translated into proteins, CircAXIN1 encodes AXIN1 protein and promotes gastric cancer.

Synthetic circular RNA is extremely stable since the lack of 5′ and 3′ ends protect circular RNA from exonuclease degradation. Further, circular RNA can be translated for sustained periods both in vitro and in vivo. Circular RNAs do not require storage at ultra-low temperatures such as −80° C. or −20° C. required by linear RNAs. Accordingly, described by the present invention are methods of making circular RNA for therapeutic use, including mRNA vaccines. Circular RNAs for expressing proteins of interest can comprise at least one internal ribosome entry site (IRES) element and a polynucleotide that encodes the protein of interest, and optionally one or more non-coding regions, wherein the IRES is operably linked to the coding polynucleotide.

### Methods of Making Circular RNA

In some aspects, provided herein is a method of making circular RNA, the method comprising: (i) contacting a linear RNA comprising a 5′ end and a 3′ end with a RNA ligase and a DNA splint comprising a region complementary to the 5′ end of the linear RNA and a region complementary to the 3′ end of the linear RNA, wherein the DNA splint binds the linear RNA and the ligase joins the 5′ end and the 3′ end; and (ii) removing the DNA splint; thereby generating circular RNA.

In some aspects, provided herein is a method of joining two linear RNA molecules by binding a DNA splint, the method comprising: (i) contacting the two linear RNA molecules, each comprising a 5′ end and a 3′ end with a RNA ligase and a DNA splint comprising a first region of sequence complementary to the 3′ end of the first linear RNA and a second region of sequence complementary to the 5′ end of the second linear RNA, wherein the DNA splint binds the linear RNA and the ligase joins the 3′ end of the first linear RNA and the 5′ end of the second linear RNA; and (ii) removing the DNA splint; thereby generating a single linear RNA.

In some embodiments, the sprinted ligation method of the present invention further comprises purifying the circular or linear RNA products.

In some embodiments, provided herein is a method of manufacturing RNA comprising a step of circularizing RNA by the methods described herein.

In some aspects, provided herein is a method of making circular RNA, the method comprising: contacting two linear RNA molecules, each comprising a 5′ end and a 3′ end with a RNA ligase, and two DNA splints, wherein the first DNA splint comprises two regions of sequences complementary to the 3′ end of the first linear RNA and to the 5′ end of the second linear RNA, wherein the second DNA splint comprises two regions of sequences complementary to the 3′ end of the second linear RNA and to the 5′ end of the first linear RNA, such that the two DNA splints bind the linear RNA molecules and the ligase joins the ends of the linear RNA molecules; and (ii) removing the DNA splints; thereby generating a circular RNA.

In some embodiments, more than two (e.g., 3, 4 or more) linear RNA molecules are ligated to form a circular RNA.

In some embodiments, one linear RNA is an Internal Ribosome Entry Site (IRES) element, and a second linear RNA is a messenger RNA that encodes protein.

In some embodiments, the linear and/or circular polynucleotide described herein, includes from about 30 to about 100,000 nucleotides (e.g., from 30 to 50, from 30 to 100, from 30 to 250, from 30 to 500, from 30 to 1,000, from 30 to 1,500, from 30 to 3,000, from 30 to 5,000, from 30 to 7,000, from 30 to 10,000, from 30 to 25,000, from 30 to 50,000, from 30 to 70,000, from 100 to 250, from 100 to 500, from 100 to 1,000, from 100 to 1,500, from 100 to 3,000, from 100 to 5,000, from 100 to 7,000, from 100 to 10,000, from 100 to 25,000, from 100 to 50,000, from 100 to 70,000, from 100 to 100,000, from 500 to 1,000, from 500 to 1,500, from 500 to 2,000, from 500 to 3,000, from 500 to 5,000, from 500 to 7,000, from 500 to 10,000, from 500 to 25,000, from 500 to 50,000, from 500 to 70,000, from 500 to 100,000, from 1,000 to 1,500, from 1,000 to 2,000, from 1,000 to 3,000, from 1,000 to 5,000, from 1,000 to 7,000, from 1,000 to 10,000, from 1,000 to 25,000, from 1,000 to 50,000, from 1,000 to 70,000, from 1,000 to 100,000, from 1,500 to 3,000, from 1,500 to 5,000, from 1,500 to 7,000, from 1,500 to 10,000, from 1,500 to 25,000, from 1,500 to 50,000, from 1,500 to 70,000, from 1,500 to 100,000, from 2,000 to 3,000, from 2,000 to 5,000, from 2,000 to 7,000, from 2,000 to 10,000, from 2,000 to 25,000, from 2,000 to 50,000, from 2,000 to 70,000, and from 2,000 to 100,000 nucleotides.

In some embodiments, viral vectors are used to package the constructs for producing the linear and/or circular polynucleotides described herein. In some embodiments, AAV vectors are used to construct the linear and/or circular polynucleotides. In other embodiments, non-viral vectors such as plasmids, cosmids and artificial chromosomes are used to construct the circular polynucleotides.

In some embodiments, the linear RNA and the DNA splint are contacted at a ratio of 4:1, 3:1, 2:1 or 1:1 ratio. In some embodiments, the linear RNA and the DNA splint are contacted in a 1:4, 1:3 or 1:2 ratio. In some embodiments, the linear RNA and the DNA splint are contacted in a 1:1 ratio.

In some embodiments. A method for generating a circular RNA comprising joining two or more linear RNA fragments using one or more DNA splints to generate a linear RNA precursor. The linear RNA precursor can then be circularized using the DNA-splint based ligation as described herein. The two or more RNA fragments can be any RNA sequences.

Exemplary linear RNA molecules include an internal ribosome entry site (IRES) element, a coding sequence that encodes a polypeptide of interest (e.g., a therapeutic protein, or an immunogen), a microRNA, an aptamer, an active RNA sequence, a polyA sequence, a translation regulatory sequence, a signal sequence, a promoter and the like.

### Splint

The present invention provides a splint design to increase ligation and circularization efficiency. In general, the splint can be any types of nucleic acids, such as DNA, RNA, 2′-OMe RNA, a DNA: RNA hybrid, PMO, LNA, and the like. In some embodiments, the splint is single-stranded.

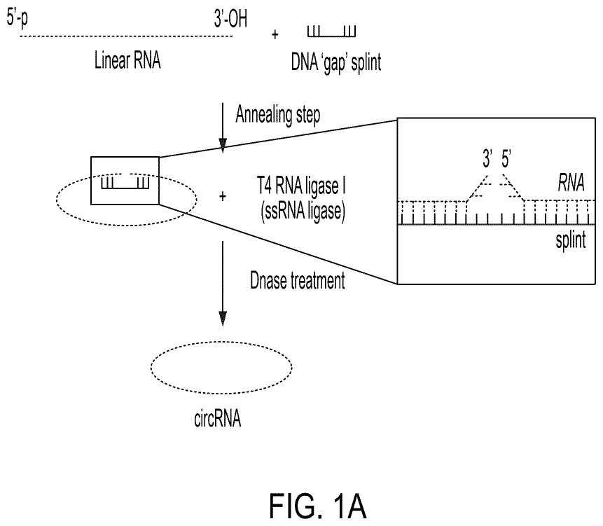

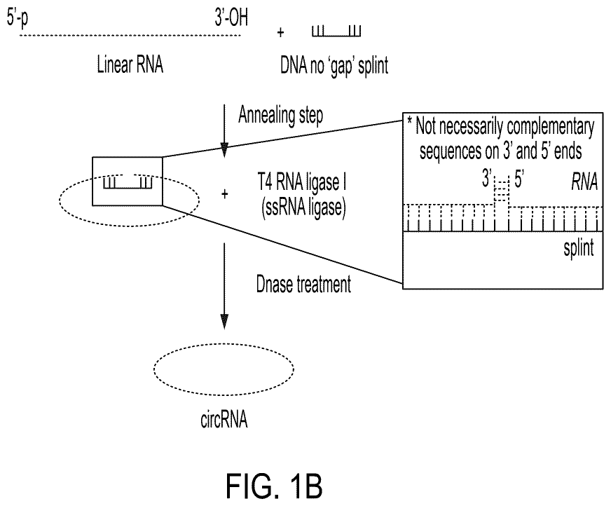

In one aspect, the splint is a DNA splint that is designed to increase circularization efficiency to greater than 60%. The DNA splint of the present invention has long complementary sequence regions and a gap across from single stranded RNA. By careful selection of the length of complementary sequence regions s and the residues comprised in the gap, the inventors were able to increase circularization (e.g., FIG. 1A). In an alternative design, the inventors also demonstrated that a splint with longer complementary sequence regions has no gap between the regions can increase circularization, the no-gap splint will generate overhangs of about 1-20 nucleotides at the 5′ end and 3 end of the RNA, i.e., a single stranded region (e.g., FIG. 1B).

In some embodiments, the DNA splint is single-stranded DNA.

As used herein, DNA splint includes a RNA:DNA hybrid, i.e., in some embodiments, the DNA splint comprises uridine nucleotides.

In some embodiments, the splint described here can be a RNA splint.

In some embodiments, the DNA splint is modified to facilitate removal after ligation. In some embodiments, the DNA splint is biotinylated.

### Complementary Sequences

The DNA splint of the present invention comprises long sequence regions complementary s to the 5′ end of the linear RNA and to the 3′ end of the linear RNA. In some embodiments, a region of sequence complementary to the 5′ end of the linear RNA and/or to the 3′ end of the linear RNA is at least 50 nucleotides long, i.e., each complementary sequence region, one with complementarity to the 5′ end of the linear RNA and one with complementarity to the 3′ end of the linear RNA is at least 50 nucleotides long.

In some embodiments, a region of sequence complementary to the 5′ end of the linear RNA and/or to the 3′ end of the linear RNA is between 10 to 500 nucleotides long. In some embodiments, a region of sequence complementary to the 5′ end of the linear RNA and/or to the 3′ end of the linear RNA is between 20 to 120 nucleotides long.

In some embodiments, the complementary sequence region is between about 50 to 500 nucleotides. In some embodiments, the complementary sequence region is about 50 nucleotides. In some embodiments, the complementary sequence region is about 100 nucleotides. In some embodiments, the complementary sequence region is about 150 nucleotides. In some embodiments, the complementary sequence region is about 200 nucleotides. In some embodiments, the homology arm is about 250 nucleotides. In some embodiments, the complementary sequence region is about 300 nucleotides. In some embodiments, the complementary sequence region is about 350 nucleotides. In some embodiments, the complementary sequence region is about 400 nucleotides. In some embodiments, the complementary sequence region is about 450 nucleotides. In some embodiments, the complementary sequence region is about 500 nucleotides. In some embodiments, the complementary sequence region is greater than 500 nucleotides.

In some embodiments, the region of sequence complementary to the 5′ end of the linear RNA and/or to the 3′ end of the linear RNA is between 1% to 10% of the length of the linear RNA. In some embodiments, the region of sequence complementary to the 5′ end of the linear RNA and/or to the 3′ end of the linear RNA is 1% of the length of the linear RNA. In some embodiments, the region of sequence complementary to the 5′ end of the linear RNA and/or to the 3′ end of the linear RNA is 2% of the length of the linear RNA. In some embodiments, the region of sequence complementary to the 5′ end of the linear RNA and/or to the 3′ end of the linear RNA is 3% of the length of the linear RNA. In some embodiments, the region of sequence complementary to the 5′ end of the linear RNA and/or to the 3′ end of the linear RNA is 4% of the length of the linear RNA. In some embodiments, the region of sequence complementary to the 5′ end of the linear RNA and/or to the 3′ end of the linear RNA is 5% of the length of the linear RNA. In some embodiments, the region of sequence complementary to the 5′ end of the linear RNA and/or to the 3′ end of the linear RNA is 6% of the length of the linear RNA. In some embodiments, the region of sequence complementary to the 5′ end of the linear RNA and/or to the 3′ end of the linear RNA is 7% of the length of the linear RNA. In some embodiments, the region of sequence complementary to the 5′ end of the linear RNA and/or to the 3′ end of the linear RNA is 8% of the length of the linear RNA. In some embodiments, the region of sequence complementary to the 5′ end of the linear RNA and/or to the 3′ end of the linear RNA is 9% of the length of the linear RNA. In some embodiments, the region of sequence complementary to the 5′ end of the linear RNA and/or to the 3′ end of the linear RNA is 10% of the length of the linear RNA. In some embodiments, the region of sequence complementary to the 5′ end of the linear RNA and/or to the 3′ end of the linear RNA is 11% of the length of the linear RNA. In some embodiments, the region of sequence complementary to the 5′ end of the linear RNA and/or to the 3′ end of the linear RNA is 12% of the length of the linear RNA. In some embodiments, the region of sequence complementary to the 5′ end of the linear RNA and/or to the 3′ end of the linear RNA is 13% of the length of the linear RNA. In some embodiments, the region of sequence complementary to the 5′ end of the linear RNA and/or to the 3′ end of the linear RNA is 14% of the length of the linear RNA. In some embodiments, the region of sequence complementary to the 5′ end of the linear RNA and/or to the 3′ end of the linear RNA is 15% of the length of the linear RNA. In some embodiments, the region of sequence complementary to the 5′ end of the linear RNA and/or to the 3′ end of the linear RNA is 16% of the length of the linear RNA. In some embodiments, the region of sequence complementary to the 5′ end of the linear RNA and/or to the 3′ end of the linear RNA is 17% of the length of the linear RNA. In some embodiments, the region of sequence complementary to the 5′ end of the linear RNA and/or to the 3′ end of the linear RNA is 18% of the length of the linear RNA. In some embodiments, the region of sequence complementary to the 5′ end of the linear RNA and/or to the 3′ end of the linear RNA is 19% of the length of the linear RNA. In some embodiments, the region of sequence complementary to the 5′ end of the linear RNA and/or to the 3′ end of the linear RNA is 20% of the length of the linear RNA.

In some embodiments, the region is symmetric across the gap, i.e., each region on either side of the gap is the same length. In some embodiments, the region is asymmetric across the gap, i.e., each region on either side of the gap is of different length.

In some embodiments, the region of sequence complementary to the 5′ end of the linear RNA and/or to the 3′ end of the linear RNA has a 5′ overhang and/or a 3′ overhang. In some embodiments, the 5′ overhang is between 1-20 nucleotides, or between 2-10 nucleotides in length. In some embodiments, the 5′ overhang is 2 nucleotides in length. In some embodiments, the 5′ overhang is 3 nucleotides in length. In some embodiments, the 5′ overhang is 4 nucleotides in length. In some embodiments, the 5′ overhang is 5 nucleotides in length In some embodiments, the 5′ overhang is 6 nucleotides in length In some embodiments, the 5′ overhang is 7 nucleotides in length In some embodiments, the 5′ overhang is 8 nucleotides in length In some embodiments, the 5′ overhang is 9 nucleotides in length In some embodiments, the 5′ overhang is 10 nucleotides in length In some embodiments, the 5′ overhang is 11 nucleotides in length. In some embodiments, the 5′ overhang is 12 nucleotides in length. In some embodiments, the 5′ overhang is 13 nucleotides in length. In some embodiments, the 5′ overhang is 14 nucleotides in length. In some embodiments, the 5′ overhang is 15 nucleotides in length. In some embodiments, the 5′ overhang is 16 nucleotides in length. In some embodiments, the 5′ overhang is 17 nucleotides in length. In some embodiments, the 5′ overhang is 18 nucleotides in length. In some embodiments, the 5′ overhang is 19 nucleotides in length. In some embodiments, the 5′ overhang is 20 nucleotides in length.

In some embodiments, the 3′ overhang is between 1-20 nucleotides, or between 2-10 nucleotides in length. In some embodiments, the 3′ overhang is 2 nucleotides in length. In some embodiments, the 3′ overhang is 3 nucleotides in length. In some embodiments, the 3′ overhang is 4 nucleotides in length. In some embodiments, the 3′ overhang is 5 nucleotides in length. In some embodiments, the 3′ overhang is 6 nucleotides in length. In some embodiments, the 3′ overhang is 7 nucleotides in length. In some embodiments, the 3′ overhang is 8 nucleotides in length. In some embodiments, the 3′ overhang is 9 nucleotides in length. In some embodiments, the 3′ overhang is 10 nucleotides in length. In some embodiments, the 3′ overhang is 11 nucleotides in length. In some embodiments, the 3′ overhang is 12 nucleotides in length. In some embodiments, the 3′ overhang is 13 nucleotides in length. In some embodiments, the 3′ overhang is 14 nucleotides in length. In some embodiments, the 3′ overhang is 15 nucleotides in length. In some embodiments, the 3′ overhang is 16 nucleotides in length. In some embodiments, the 3′ overhang is 17 nucleotides in length. In some embodiments, the 3′ overhang is 18 nucleotides in length. In some embodiments, the 3′ overhang is 19 nucleotides in length. In some embodiments, the 3′ overhang is 20 nucleotides in length.

### Gap

In some embodiments, the DNA splint comprises a gap. In some embodiments, the gap is single-stranded. In some embodiments, the single-stranded gap in the DNA splint is across from single-stranded RNA nucleotides.

In some embodiments, the gap is between 2-6 nucleotides in length. In some embodiments, the gap is 2 nucleotides in length. In some embodiments, the gap is 3 nucleotides in length. In some embodiments, the gap is 4 nucleotides in length. In some embodiments, the gap is 5 nucleotides in length. In some embodiments, the gap is 6 nucleotides in length.

In some embodiments, the gap is between 7-15 nucleotides in length. In some embodiments, the gap is 7, 8, 9, 10, 11, 12, 13, 14 or 15 nucleotides in length. In some embodiments, the gap is 7 nucleotides in length. In some embodiments, the gap is 8 nucleotides in length. In some embodiments, the gap is 9 nucleotides in length. In some embodiments, the gap is 10 nucleotides in length. In some embodiments, the gap is 11 nucleotides in length. In some embodiments, the gap is 12 nucleotides in length. In some embodiments, the gap is 13 nucleotides in length. In some embodiments, the gap is 14 nucleotides in length. In some embodiments, the gap is greater than 15 nucleotides in length.

In some embodiments, the gap is a single nucleotide.

In some embodiments, the gap is 4 nucleotides in length.

In some embodiments, the gap is comprised of adenine, thymine, guanine, cytosine and/or uridine nucleotides. In some embodiments, the gap is comprised of adenine nucleotides. In some embodiments, the gap is comprised of thymine nucleotides. In some embodiments, the gap is comprised of guanine nucleotides. In some embodiments, the gap is comprised of cytosine nucleotides. In some embodiments, the gap is comprised of uridine nucleotides.

In some embodiments, the gap is a homopolymeric stretch of nucleotides. In some embodiments, the gap is a homopolymeric stretch of adenine nucleotides. In some embodiments, the gap is a homopolymeric stretch of thymine nucleotides. In some embodiments, the gap is a homopolymeric stretch of guanine nucleotides. In some embodiments, the gap is a homopolymeric stretch of cytosine nucleotides. In some embodiments, the gap is a homopolymeric stretch of uridine nucleotides.

In some embodiments, the gap is comprised of adenine nucleotides. In some embodiments, the gap is comprised of thymine nucleotides. In some embodiments, the gap is comprised of guanine nucleotides. In some embodiments, the gap is comprised of cytosine nucleotides. In some embodiments, the gap is comprised of uridine nucleotides.

In some embodiments, the gap is comprised of one or more modified nucleotides and/or abasic sites. In some embodiments, the gap is comprised of modified nucleotides. In some embodiments, the modified nucleotide is pseudouridine, N1-methylpseudouridine (N1m-Ψ), and/or 5-methoxyuridine (5moU). In some embodiments, the gap is comprised of one or more abasic sites. In some embodiments, the gap is comprised of one or more apurinic sites. In some embodiments, the gap is comprised of one or more apyrimidinic sites.

In some embodiments, the DNA splint comprises no gap. In this design, the splint after binding to an RNA through the complementary sequence regions generate overhangs of at least 1-20 nucleotides at the 5′ and/or 3′ end (e.g., FIG. 1B). In some embodiments, the overhangs at the 5′ end and/or 3′ end is 1 nucleotide in length. In some embodiments, the overhangs at the 5′ end and/or 3′ end is 2 nucleotides in length. In some embodiments, the overhangs at the 5′ end and/or 3′ end is 3 nucleotides in length. In some embodiments, the overhangs at the 5′ end and/or 3′ end is 4 nucleotides in length. In some embodiments, the overhangs at the 5′ end and/or 3′ end is 5 nucleotides in length. In some embodiments, the overhangs at the 5′ end and/or 3′ end is 6 nucleotides in length. In some embodiments, the overhangs at the 5′ end and/or 3′ end is 7 nucleotides in length. In some embodiments, the overhangs at the 5′ end and/or 3′ end is 8 nucleotides in length. In some embodiments, the overhangs at the 5′ end and/or 3′ end is 9 nucleotides in length. In some embodiments, the overhangs at the 5′ end and/or 3′ end is 10 nucleotides in length. In some embodiments, the overhangs at the 5′ end and/or 3′ end is 11 nucleotides in length. In some embodiments, the overhangs at the 5′ end and/or 3′ end is 12 nucleotides in length. In some embodiments, the overhangs at the 5′ end and/or 3′ end is 13 nucleotides in length. In some embodiments, the overhangs at the 5′ end and/or 3′ end is 14 nucleotides in length. In some embodiments, the overhangs at the 5′ end and/or 3′ end is 15 nucleotides in length. In some embodiments, the overhangs at the 5′ end and/or 3′ end is 16 nucleotides in length. In some embodiments, the overhangs at the 5′ end and/or 3′ end is 17 nucleotides in length. In some embodiments, the overhangs at the 5′ end and/or 3′ end is 18 nucleotides in length. In some embodiments, the overhangs at the 5′ end and/or 3′ end is 19 nucleotides in length. In some embodiments, the overhangs at the 5′ end and/or 3′ end is 20 nucleotides in length.

polyA Sequence

In some embodiments, the splint further contains a homopolymer of 10-100, 20-80, or 20-50, or 10-30 Adenines at the 5′ end or 3′ end of the splint. The poly(A) stretch can include 10As, 15As, 20As, 25As, 30As, 35As, 40As, 45As, or 50As. The ploy (A)+ splint can be removed by a homopolymer of T nucleotides after ligation, e.g., a dT-conjugated resin.

### RNA Ligase

According to the present invention, a RNA ligase enzyme is used to join 5′ and 3′ ends or termini of RNA that are brought together by a DNA splint that shares regions of homology with the 5′ end and 3′ end, respectively. In some embodiments, the ligase is a RNA ligase that is used to join 5′ end and 3′ end of a single RNA molecule to circularize it. In some embodiments, the ligase is a RNA ligase that is used to join the 5′ end of one RNA molecule and the 3′ end of another RNA molecule. RNA ligase enzymes, termed 5′-3′ RNA ligases join 5′-phosphate (5′-PO₄) and 3′-hydroxyl (3′-OH) termini of RNA via a three-step mechanism: (a) RNA ligase undergoes adenosine 5′-triphosphate (ATP)-dependent auto-AMPylation or auto-adenylation at the catalytic lysine residue, (b) AMP is subsequently transferred from the ligase-(lysyl-N)-AMP to the 5′-PO₄ end of RNA (pRNA) to yield the RNA-adenylate intermediate (AppRNA), and (c) the two RNA ends are ligated by a phosphodiester bond upon nucleophilic attack of the 3′-OH to the AppRNA, liberating adenosine monophosphate (AMP).

In some embodiments, the ligase is T4 RNA ligase 1. T4 RNA ligase 1 catalyzes the ATP-dependent covalent joining of single-stranded 5′-phosphoryl termini of DNA or RNA to single-stranded 3′-hydroxyl termini of DNA or RNA.

In some embodiments, the ligase is T4 RNA ligase 2. T4 RNA ligase 2 also catalyzes the joining of a 3′-hydroxyl terminus of RNA to a 5′-phosphorylated RNA or DNA; however, unlike T4 RNA ligase 1, this enzyme prefers double-stranded substrates. In some embodiments, a ligase ligates dsRNA nicks and ssRNA to ssRNA. In some embodiments, a ligase ligates the 3′ end of RNA or DNA to a 5′ pDNA when annealed to a RNA complement, and the 3′ end of RNA to a 5′ pRNA when annealed to a DNA complement.

A truncated form of T4 RNA ligase 2 requires a pre-adenylated substrate for ligation. In some embodiments, the RNA ligase is a T4 RNA Ligase 2, truncated K227Q ligase. In some embodiments, the truncated K227Q ligase joins a 3′ end of ssRNA to 5′ adenylated ssDNA or 5′ adenylated ssRNA.

In some embodiments, the RNA ligase is a truncated KQ ligase that ligates ssRNA to preadenylated ends. In some embodiments, the 5′ end is preadenylated. In some embodiments, the truncated KQ ligase joins a 3′ end of ssRNA to 5′ adenylated ssDNA or 5′ adenylated ssRNA. In some embodiments, 5′ adenylated RNA (5′ AppRNA) removes the need to dephosphorylate RNA prior to ligation and prevents undesirable ligation products.

In some embodiments, the ligase is a RtcB Ligase, Thermo-stable 5′ App DNA/RNA Ligase, ElectroLigase, T4 DNA Ligase, T3 DNA Ligase, T7 DNA Ligase, Taq DNA Ligase, SplintR Ligase E. coli DNA Ligase, 9°N DNA Ligase, CircLigase, CircLigase II, DNA Ligase I, DNA Ligase III, or DNA Ligase IV. In some embodiments, the ligase is a SplintR ligase which ligates the 3′ end of RNA to 5′ phosphate end of DNA when annealed to complementary DNA or RNA.

In some embodiments, the RtcB ligase ligates 3′ phosphate or 2′, 3′-cyclic phosphate to the 5′ OH of ssRNA.

In some embodiments, the RNA ligase is thermostable 5′ AppDNA/RNA ligase which ligates 3′ end of ssRNA or ssDNA to a 5′ adenylated ssDNA or 5′ adenylated ssRNA.

### Removal of DNA Splint

In some embodiments, the DNA splint is a removed after ligation by enzymatic digestion with a DNA nuclease. In some embodiments, the DNA nuclease is DNase I. In some embodiments, wherein the DNA splint is biotinylated, the DNA splint is removed by denaturing and binding to streptavidin, which binds to biotin with very high affinity.

In some embodiments, the DNA splint comprises abasic sites, uridine or modified nucleotides that destabilize the splint and the splint is removed by denaturation. The DNA splint is denatured by heat, alkaline pH or enzymatic methods. In some embodiments, DNA denaturation by heat occurs in a process that is similar to melting and strand separation of the RNA:DNA hybrid. In some embodiments, sodium hydroxide or increased hydroxyl groups at an alkaline pH are also used to denature DNA. The OH— groups remove protons and break hydrogen bonds between bases in an RNA:DNA hybrid.

In some embodiments, enzymatic methods are used to remove the DNA splint. In some embodiments, removal of the DNA splint comprises digesting with a DNA nuclease. In some embodiments, the DNA nuclease is a DNA exonuclease. In some embodiments, the DNA nuclease is a DNase I.

In some embodiments, the splint is removed using reverse splint. As used herein, the term “reverse splint” refers to an nucleic acid that comprises a sequence at least 70%, 80%, 90%, 95% or more complementary to the splint. In some embodiments, the reverse splint comprises a poly(A) sequence that is about 5-250 adenines (As) in length, at the 5′ end, or 3′end of the reverse splint. In some embodiments, the reverse splint comprises about a poly(A) stretch having 10-100As, or 20-80 As, or 30-50 As. As non-limiting examples, the reverse splint comprises about a poly(A) stretch having 25As, 30As, 35As, 40As, 45As, 50As, 60As, 70As, 80As, 90As, 100As, 150As or 200As. In some embodiments, the reverse splint is used at a ratio with the splint of 1:15, 1:10, 1:8, or 1:5. In one example, the reverse splint is used at a ratio with the splint of 1:10. The splint can be released from RNA by heating the reaction and the released splint binds to the reverse splint to be removed, for example using oligo-dT that binds to the poly(A) sequence of the reverse splint.

In some embodiments, the reverse splint is biotinylated.

Similarly, the reverse splint can be any type of nucleic acids such as DNA, RNA, 2′-OMe RNA, a DNA: RNA hybrid, PMO, LNA, and the like

### Removal of Un-Ligated RNA

In some embodiments, the splinted ligation method described herein comprises removal of un-ligated RNA from the ligation reaction, thereby increasing purity of circular RNA. In some examples, the method includes a step to treat the ligation reaction with a polyA polymerase, which adds a poly(A) tail to free 3′-OH of an un-ligated RNA. The poly(A) tailed RNA can be removed with oligo-dT, e.g., a dT-column.

### Linear RNA

In some embodiments, the linear RNA is synthetic RNA. In some embodiments, the linear RNA is chemically synthesized. In some embodiments, RNA synthesis is carried out in synthesizer machines using nucleotide triphosphate derivatives known as phosphoramidites, which are building blocks of linear oligonucleotides. Nucleoside phosphoramidites use inert substituents to protect reactive moieties such as hydroxyl and amino groups from undesirable reactions and promote phosphodiester bond formation leading to greater homogenous yields. Once synthesis is complete, these groups are removed to generate RNA oligonucleotides of high purity. However, these are typically small RNAs, of up to 70-80 nucleotides in length, beyond which the method is associated with low yields and high costs.

In some embodiments, the linear RNA is in vitro transcribed RNA. An in vitro transcription (IVT) reaction typically comprises a double-stranded DNA (dsDNA) template, ribonucleotide triphosphates, and a DNA-dependent RNA polymerase. In some embodiments, the DNA-dependent RNA polymerase is derived from bacteriophage. In some embodiments, the DNA-dependent RNA polymerase is a T7 RNA polymerase, SP6 RNA polymerase, or T3 RNA polymerase.

The DNA template contains a promoter sequence to which the polymerase binds and catalyzes downstream transcription. In some embodiments, the promoter is about 20-40 nucleotides long. In some embodiments, the DNA template is a double-stranded PCR product. In some embodiments, the DNA template is a linearized plasmid containing a promoter upstream of the DNA sequence to be transcribed.

Nucleotide triphosphates used during in vitro transcription (IVT) result in a single-stranded RNA containing guanine nucleotide triphosphate (GTP) at its 5′ end. Since enzymatic intramolecular ligation of RNA requires a 5′ monophosphate, an excess molar ratio of GMP to GTP is used during the IVT reaction.

In some embodiments, the in vitro transcription is in the presence of GMP. In some embodiments, the in vitro transcription is in 5 fold excess GMP as compared to GTP.

In some embodiments, if GTP alone was used to prime transcription, monophosphorylation is achieved by treatment of the RNA with a phosphatase followed by a kinase, or by treatment with a pyrophosphatase. In some embodiments, the in vitro transcribed RNA is treated with phosphatase.

In some embodiments, the in vitro transcribed RNA is treated with kinase.

In some embodiments, RppH converts triphosphate to monophosphate.

In some embodiments, the 5′ end is a 5′ monophosphate.

In some embodiments, the 3′ end is a 3′ hydroxyl.

Enzymatic ligation of a monophosphorylated RNA is achieved using T4 DNA ligase, T4 RNA ligase 1, or T4 RNA ligase 2. A DNA splint, or in some embodiments, an RNA splint, is used to bring the terminal ends of the RNA together. Compared to chemical synthesis, enzymatic synthesis allows for the generation of much larger linear RNAs (kb in length).

In some embodiments, the linear RNA is messenger RNA. In some embodiments, the linear RNA encodes one or more polypeptide(s). In some embodiments, the coding sequence element may encode two or more proteins of interest. The two or more proteins of interest have the same amino acid sequences; alternatively, the two or more proteins of interest have different amino acid sequences. The protein of interest may be one known in the art and/or described herein. In this context, the circular RNA may act as a messenger RNA (mRNA), having the same encoding function as its linear RNA counterpart.

The protein in some cases is a recombinant polypeptide. A recombinant polypeptide may include, but is not limited to, full-length polypeptides, a plurality of polypeptides or fragments of polypeptides, which may be independently encoded by one or more nucleic acids, a plurality of nucleic acids, fragments of nucleic acids or variants of any of the aforementioned. The coding sequence may be sufficient to encode a polypeptide of at least 10 amino acids in length, e.g., 10 to 5000 amino acids in length, or 10-1000 amino acids in length, or 50-2000 amino acids in length, or 30-3000 amino acids in length, or 100-1000 amino acids in length, or 100-3000 amino acids in length, or 200-1000 amino acids in length, or 200-500 amino acids in length, or 500-5000 amino acids in length, or 500-4000 amino acids in length, or 500-1500 amino acids in length, or 1000-5000 amino acids in length. The coding sequence may encode a polypeptide of 100 amino acids, 150 amino acids, 200 amino acids, 250 amino acids, 300 amino acids, 350 amino acids, 400 amino acids, 450 amino acids, 500 amino acids, 550 amino acids, 600 amino acids, 650 amino acids, 700 amino acids, 750 amino acids, 800 amino acids, 850 amino acids, 900 amino acids, 950 amino acids, 1000 amino acids, 2000 amino acids, 3000 amino acids, 4000 amino acids or 5000 amino acids in length.

In some embodiments, the linear RNA and/or circular RNA comprises one or more modified nucleotides. In some embodiments, the linear RNA and/or circular RNA comprises only unmodified nucleotides.

In some embodiments, the linear RNA and/or circular RNA does not comprise an intron.

In some embodiments, the linear RNA and/or circular RNA does not comprise homology arms.

In some embodiments, the linear RNA and/or circular RNA is between 100 bp to 10 kb in length. In some embodiments, the linear RNA and/or circular RNA is between 100 bp to 500 bp in length. In some embodiments, the linear RNA and/or circular RNA is between 500 bp to 10 kb in length. Generally, the linear RNA and/or circular RNA may be greater than 30 nucleotides in length, e.g., at least or greater than about 35, 40, 45, 50, 55, 60, 70, 80, 90, 100, 120, 140, 160, 180, 200, 250, 300, 350, 400, 450, 500, 600, 700, 800, 900, 1,000, 1, 100, 1,200, 1,300, 1,400, 1,500, 1,600, 1,700, 1,800, 1,900, 2,000, 2250, 2,500, 2, 750 and 3,000, 3,500, 4,000, 4,500, 5,000, 5,500, 6,000, 6,500, 7,000, 7,500, 8,000, 8,500, 9,000, 9,500, 10,000, 20,000, 30,000, 40,000, 50,000, 60,000, 70,000, 80,000, 90,000 or up to and including 100,000 nucleotides.

In some embodiments, the linear RNA and/or circular RNA encodes a therapeutic protein (e.g., a chimeric antigen receptor or a gene therapy). Therapeutic proteins include peptides or proteins, which are beneficial for the treatment of a disease or which improves the condition of an individual. Particularly, therapeutic proteins play an important role in the creation of therapeutic agents that could modify and repair genetic errors, destroy cancer cells or pathogen infected cells, treat immune system disorders, treat metabolic or endocrine disorders, among other functions. Therapeutic proteins also include adjuvant proteins, therapeutic antibodies, and recombinant proteins for enzyme replacement therapy. In some embodiments, therapeutic proteins further include recombinant or chimeric proteins such as chimeric antigen receptors (CARs) and TCRs.

In some embodiments, the therapeutic protein is a human protein.

In some embodiments, the linear RNA and/or circular RNA encodes an immunogen (e.g., an immunogen derived from an infectious pathogen, and a cancer specific antigen). In some embodiments, the antigen is a pathogenic antigen such as a virus derived antigen, a cancer derived antigen, an allergenic antigen or an autoimmune antigen. In some embodiments, the antigen is a pathogenic antigen or a fragment, variant or derivative thereof. Such pathogenic antigens are derived from pathogenic organisms, in particular parasites, bacteria, viruses, fungi or other multicellular pathogenic organisms, which evoke an immunological reaction in a subject. In some embodiments, the antigen is a viral antigen derived from Influenza virus, respiratory syncytial virus (RSV), Herpes simplex virus (HSV), human Papilloma virus (HPV), Human immunodeficiency virus (HIV), Plasmodium, Staphylococcus aureus, Dengue virus, Chlamydia trachomatis, Cytomegalovirus (CMV), Hepatitis B virus (HBV), Mycobacterium tuberculosis, Rabies virus, coronaviruses and Yellow Fever Virus. In some embodiments, the antigen is a cancer specific antigen, a fragment, variant or derivative thereof. The term “cancer” denotes a malignant neoplasm that has undergone characteristic anaplasia with loss of differentiation, increased rate of growth, invasion of surrounding tissue, and is capable of metastasis. The term “cancer” shall be taken to include a disease that is characterized by uncontrolled growth of cells within a subject. In some embodiments, the terms “cancer” and “tumor” are used interchangeably. In some embodiments, the term “tumor” refers to a benign or non-malignant growth.

Exemplary cancer specific antigens include 5T4, 707-AP, 9D7, AFP, AlbZIP HPG1, alpha-5-beta-1-integrin, alpha-5-beta-6-integrin, alpha-actinin-4/m, alpha-methylacyl-coenzyme A racemase, ART-4, ARTC1/m, B7H4, BAGE-1, BCL-2, bcr/abl, beta-catenin/m, BING-4, BRCA1/m, BRCA2/m, CA 15-3/CA 27-29, CA 19-9, CA72-4, CA125, caireticulin, CAMEL, CASP-8/m, cathepsin B, cathepsin L, CD19, CD20, CD22, CD25, CDE30, CD33, CD4, CD52, CD55, CD56, CD80, CDC27/m, CDK4/m, CDKN2A/m, CEA, CLCA2, CML28, CML66, COA-1/m, coactosin-like protein, collage XXIII, COX-2, CT-9/BRD6, Cten, cyclin B1, cyclin D1, cyp-B, CYPB1, DAM-10, DAM-6, DEK-CAN, EFTUD2/m, EGFR, ELF2/m, EMMPRIN, EpCam, EphA2, EphA3, ErbB3, ETV6-AML1, EZH2, FGF-5, FN, Frau-1, G250, GAGE-1, GAGE-2, GAGE-3, GAGE-4, GAGE-5, GAGE-6, GAGE7b, GAGE-8, GDEP, GnT-V, gp100, GPC3, GPNMB/m, HAGE, HAST-2, hepsin, Her2/neu, HERV-K-MEL, HLA-A*0201-R17I, HLA-A11/m, HLA-A2/m, HNE, homeobox NKX3.1, HOM-TES-14/SCP-1, HOM-TES-85, HPV-E6, HPV-E7, HSP70-2M, HST-2, hTERT, iCE, IGF-1R, IL-13Ra2, IL-2R, IL-5, immature laminin receptor, kallikrein-2, kallikrein-4, Ki67, KIAA0205, KIAA0205/m, KK-LC-1, K-Ras/m, LAGE-A1, LDLR-FUT, MAGE-A1, MAGE-A2, MAGE-A3, MAGE-A4, MAGE-A6, MAGE-A9, MAGE-A10, MAGE-A12, MAGE-B1, MAGE-B2, MAGE-B3, MAGE-B4, MAGE-B5, MAGE-B6, MAGE-B10, MAGE-B16, MAGE-B17, MAGE-C1, MAGE-C2, MAGE-C3, MAGE-D1, MAGE-D2, MAGE-D4, MAGE-E1, MAGE-E2, MAGE-F1, MAGE-H1, MAGEL2, mammaglobin A, MART-1/melan-A, MART-2, MART-2/m, matrix protein 22, MC1R, M-CSF, ME1/m, mesothelin, MG50/PXDN, MMP11, MN/CA IX-antigen, MRP-3, MUC-1, MUC-2, MUM-1/m, MUM-2/m, MUM-3/m, myosin class I/m, NA88-A, N-acetylglucosaminyltransferase-V, Neo-PAP, Neo-PAP/m, NFYC/m, NGEP, NMP22, NPM/ALK, N-Ras/m, NSE, NY-ESO-1, NY-ESO-B, OA1, OFA-iLRP, OGT, OGT/m, OS-9, OS-9/m, osteocalcin, osteopontin, p15, p190 minor bcr-abl, p53, p53/m, PAGE-4, PAI-1, PAI-2, PAP, PART-1, PATE, PDEF, Pim-1-Kinase, Pin-1, Pml/PARalpha, POTE, PRAME, PRDX5/m, prostein, proteinase-3, PSA, PSCA, PSGR, PSM, PSMA, PTPRK/m, RAGE-1, RBAF600/m, RHAMM/CD168, RU1, RU2, S-100, SAGE, SART-1, SART-2, SART-3, SCC, SIRT2/m, Sp17, SSX-1, SSX-2/HOM-MEL-40, SSX-4, STAMP-1, STEAP-1, survivin, survivin-2B, SYT-SSX-1, SYT-SSX-2, TA-90, TAG-72, TARP, TEL-AML1, TGFbeta, TGFbetaRII, TGM-4, TPI/m, TRAG-3, TRG, TRP-1, TRP-2/6b, TRP/INT2, TRP-p8, tyrosinase, UPA, VEGFR1, VEGFR-2/FLK-1, WT1 and a immunoglobulin idiotype of a lymphoid blood cell or a T cell receptor idiotype of a lymphoid blood cell, or a fragment, variant or derivative of said cancer antigen.

A cancer specific antigen is a peptide derived from a cancer specific protein. In some embodiments, the peptides can be at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more amino acid residues in length. In some embodiments, the peptides can be at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 70, 80, 90, 100, 150, 200, 250, 300, 350, 400, 450, 500, or more amino acid residues in length. In some embodiments, the peptides can be at most 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or less amino acid residues in length. In some embodiments, the peptides can be at most 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 70, 80, 90, 100, 150, 200, 250, 300, 350, 400, 450, 500 or less amino acid residues in length.

In some embodiments, the protein of interest is an antibody, a heavy chain of an antibody, a light chain of an antibody, a heavy chain variable region of an antibody, a light chain variable region of an antibody, a scFv, a VHH, a Fab fragment, and the like. In some embodiments, the antibody expressed by the circular RNA can be of any isotype, such as IgA, IgD, IgE, IgG, IgM.

In some embodiments, the antibody is a therapeutic antibody.

In some embodiments, the antibody is a bispecific antibody or multiple specific antibody.

In some embodiments, the linear RNA is a RNA vaccine.

### Modified Nucleotides

In some embodiments, the circular RNA comprises one or more modified nucleotides. In some embodiments, the linear RNA comprises one or more modified nucleotides. In some embodiments, the messenger RNA comprises one or more modified nucleotides. In some embodiments, the small RNA comprises one or more modified nucleotides. In some embodiments, the guide RNA comprises one or more modified nucleotides.

In some embodiments, the linear and/or circular RNAs of the present invention may include one, two, three, or more modifications. In some embodiments, the modified nucleotides are located in coding region(s). In some embodiments, the modified nucleotides are in the untranslated region(s).

In some embodiments, the modifications stabilize the linear and/or circular RNA and enhance resistance to degradation as compared to unmodified nucleotides. In some embodiments, modified nucleotides enhance biological functions of nucleic acid molecules, for example, increase binding to a RNA binding protein or increasing translation.

In some embodiments, the modified nucleotide is one or more of N1-methylpseudouridine, 5-methoxyuridine, N6-methyladenosine, pseudouridine or 5-methylcytosine.

In some embodiments, the modified nucleotide is N1-methylpseudouridine. In some embodiments, the modified nucleotide is 5-methoxyuridine. In some embodiments, the modified nucleotide is N6-methyladenosine. In some embodiments, the modified nucleotide is pseudouridine. In some embodiments, the modified nucleotide is 5-methylcytosine.

In some embodiments, the modified nucleotide is 100%. In some embodiments, the modified nucleotide is less than 50%. In some embodiments, the modified nucleotide is less than 20%. In some embodiments, the modified nucleotide is less than 10%.

The linear and/or circular polynucleotides of the present disclosure may contain from about 0% to about 100% modified nucleotides (either in relation to overall nucleotide content, or in relation to one or more types of nucleotide, i.e. any one or more of A, G, T/U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 85% to 95%, from 85% to 100%, from 90% to 95%, from 90% to 100%, and from 95% to 100%).

In some embodiments, the polynucleotides are 100% modified. In some embodiments, the polynucleotides are at least 50% modified, e.g., at least 50% of the nucleotides are modified. In some embodiments, the polynucleotides are at least 75% modified, e.g., at least 75% of the nucleotides are modified. In some embodiments, the polynucleotides are at least 20% modified, e.g., at least 20% of the nucleotides are modified. In some embodiments, the polynucleotides are at least 10% modified, e.g., at least 10% of the nucleotides are modified. It is to be understood that since a nucleotide (sugar, base and phosphate moiety, e.g., linkage) may each be modified, any modification to any portion of a nucleotide, or nucleoside, will constitute a modification.

In some embodiments, the modifications are structural modifications and/or chemical modifications. In some embodiments, the chemical modification is a nucleotide and/or nucleoside modification including a nucleobase modification and/or a sugar modification, and a backbone linkage modification (i.e., the internucleoside linkage, e.g., a linking phosphate, a phosphodiester linkage, and a phosphodiester backbone). In some embodiments, the structural modification includes a secondary and/or tertiary structural modification.

In some embodiments, modifications include modifications of ribonucleic acids (RNAs) to deoxyribonucleic acids (DNAs), threose nucleic acids (TNAs), glycol nucleic acids (GNAs), peptide nucleic acids (PNAs), locked nucleic acids (LNAs) or hybrids thereof.

In some embodiments, one, two, or more (optionally different) nucleoside or nucleotide modifications may be incorporated to the circular polynucleotides of the present disclosure.

In some embodiments, the linear and/or circular polynucleotide (e.g., circular RNA) comprises at least one modification described herein. In other embodiments, the circular polynucleotides comprise two, three, four, or more (optionally different) chemical modifications described herein. The modifications may be combinations of nucleobase (purine and/or pyrimidine), sugar and backbone (internucleoside) linkage modifications. The modifications may be located on one or more nucleotides of the circular polynucleotide. In some embodiments, all the nucleotides of the circular polynucleotide are chemically modified. In some embodiments, all the nucleotides of the nucleic acid sequence with a biological function are chemically modified.

In some embodiments, the polynucleotides are at least 10% modified in only one component of the nucleotide, with such component being the nucleobase, sugar, or linkage between nucleosides. For example, modifications may be made to at least 10%, 15%, 20%, 25%, 30%, 35%, 40%, 45%, 50%, 55%, 60%, 65%, 70%, 75%, 80%, 85%, 90%, 95% or 100% of the nucleobases, sugars, or linkages of a polynucleotide described herein.

In some embodiments, the linear and/or circular polynucleotides are designed with a patterned array of sugar, nucleobase or linkage modifications.

In some embodiments, the polynucleotides comprise modifications to maximize stability.

In other embodiments, the polynucleotides comprise modifications to decrease stability.

In some embodiments, the modified nucleosides and nucleotides include a modified nucleobase. Examples of nucleobases in RNA include, but are not limited to, adenine (A), guanine (G), cytosine (C), and uracil (U). Examples of nucleobases in DNA include, but are not limited to, adenine (A), guanine (G), cytosine (C), and thymine (T).

In some embodiments, the modified nucleobase is a modified uracil (U). Exemplary nucleobases and nucleosides having a modified uracil include pseudouridine (ψ), pyridin-4-one ribonucleoside, 5-aza-uridine, 6-aza-uridine, 2-thio-5-aza-uridine, 2-thio-uridine (s²U), 4-thio-uridine (s⁴U), 4-thio-pseudouridine, 2-thio-pseudouridine, 5-hydroxy-uridine (ho⁵U), 5-aminoallyl-uridine, 5-halo-uridine (e.g., 5-iodo-uridine (I⁵U) or 5-bromo-uridine (br⁵U)), 3-methyl-uridine (m³U), 5-methoxy-uridine (mo⁵U), uridine 5-oxyacetic acid (cmo⁵U), uridine 5-oxyacetic acid methyl ester (mcmo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵s²U), 5-aminomethyl-2-thio-uridine (nm⁵s²U), 5-methylaminomethyl-uridine (mnm⁵U), 5-methylaminomethyl-2-thio-uridine (mnm⁵s²U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s²U), 5-propynyl-uridine, 1-propynyl-pseudouridine, 5-taurinomethyl-uridine (tm⁵U), 1-taurinomethyl-pseudouridine, 5-taurinomethyl-2-thio-uridine (m⁵s²U), 1-taurinomethyl-4-thio-pseudouridine, 5-methyl-uridine (m⁵U, i.e., having the nucleobase deoxythymine), 1-methylpseudouridine (m¹ψ), 5-methyl-2-thio-uridine (m⁵s²U), pseudouracil (ψ), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine (also known as 1-methylpseudouridine (m¹ψ)), 3-(3-amino-3-carboxypropyl)uridine (acp³U), 1-methyl-3-(3-amino-3-carboxypropyl) pseudouridine (acp³ ψ), 5-(isopentenylaminomethyl)uridine (inm⁵U), 5-(isopentenylaminomethyl)-2-thio-uridine (inm⁵s²U), α-thio-uridine, 2′-O-methyl-uridine (Um), 5,2′-O-dimethyl-uridine (m⁵Um), 2′-O-methyl-pseudouridine (m), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), 5-(isopentenylaminomethyl)-2′-O-methyl-uridine (inm⁵Um), 1-thio-uridine, deoxythymidine, 2′-F-ara-uridine, 2′-F-uridine, 2′-OH-ara-uridine, 5-(2-carbomethoxyvinyl)uridine, and 5-[3-(1-E-propenylamino)uridine.

In some embodiments, the modified nucleobase is a modified cytosine (C). Exemplary nucleobases and nucleosides having a modified cytosine include 5-aza-cytidine, 6-aza-cytidine, pseudoisocytidine, 3-methyl-cytidine (m³C), N4-acetyl-cytidine (ac⁴C), 5-formyl-cytidine (f⁵C), N4-methyl-cytidine (m⁴C), 5-methyl-cytidine (m⁵C), 5-halo-cytidine (e.g., 5-iodo-cytidine), 5-hydroxymethyl-cytidine (hm⁵C), 1-methyl-pseudoisocytidine, pyrrolo-cytidine, pyrrolo-pseudoisocytidine, 2-thio-cytidine (s²C), 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine (k₂C), α-thio-cytidine, 2′-O-methyl-cytidine (Cm), 5,2′-O-dimethyl-cytidine (m⁵Cm), N4-acetyl-2′-O-methyl-cytidine (ac⁴Cm), N4,2′-O-dimethyl-cytidine (m⁴Cm), 5-formyl-2′-O-methyl-cytidine (f⁵Cm), N4,N4,2′-O-trimethyl-cytidine (m⁴₂Cm), 1-thio-cytidine, 2′-F-ara-cytidine, 2′-F-cytidine, and 2′-OH-ara-cytidine.

In some embodiments, the modified nucleobase is a modified adenine (A). Exemplary nucleobases and nucleosides having a modified adenine include 2-amino-purine, 2, 6-diaminopurine, 2-amino-6-halo-purine (e.g., 2-amino-6-chloro-purine), 6-halo-purine (e.g., 6-chloro-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine (m¹A), 2-methyl-adenine (m²A), N6-methyl-adenosine (m⁶A), 2-methylthio-N6-methyl-adenosine (ms²m⁶A), N6-isopentenyl-adenosine (i⁶A), 2-methylthio-N6-isopentenyl-adenosine (ms²i⁶A), N6-(cis-hydroxyisopentenyl) adenosine (io⁶A), 2-methylthio-N6-(cis-hydroxyisopentenyl) adenosine (ms²io⁶A), N6-glycinylcarbamoyl-adenosine (g⁶A), N6-threonylcarbamoyl-adenosine (t⁶A), N6-methyl-N6-threonylcarbamoyl-adenosine (m⁶t⁶A), 2-methylthio-N6-threonylcarbamoyl-adenosine (ms²g⁶A), N6,N6-dimethyl-adenosine (m⁶₂A), N6-hydroxynorvalylcarbamoyl-adenosine (hn⁶A), 2-methylthio-N6-hydroxynorvalylcarbamoyl-adenosine (ms²hn⁶A), N6-acetyl-adenosine (ac⁶A), 7-methyl-adenine, 2-methylthio-adenine, 2-methoxy-adenine, α-thio-adenosine, 2′-O-methyl-adenosine (Am), N6,2′-O-dimethyl-adenosine (m⁶Am), N6,N6,2′-O-trimethyl-adenosine (m⁶₂Am), 1,2′-O-dimethyl-adenosine (m¹Am), 2′-O-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

In some embodiments, the modified nucleobase is a modified guanine (G). Exemplary nucleobases and nucleosides having a modified guanine include inosine (I), 1-methyl-inosine (m¹I), wyosine (imG), methylwyosine (mimG), 4-demethyl-wyosine (imG-14), isowyosine (imG2), wybutosine (yW), peroxywybutosine (o₂yW), hydroxywybutosine (OHyW), undermodified hydroxywybutosine (OHyW*), 7-deaza-guanosine, queuosine (Q), epoxyqueuosine (oQ), galactosyl-queuosine (galQ), mannosyl-queuosine (manQ), 7-cyano-7-deaza-guanosine (preQ₀), 7-aminomethyl-7-deaza-guanosine (preQ₁), archaeosine (G⁺), 7-deaza-8-aza-guanosine, 6-thio-guanosine, 6-thio-7-deaza-guanosine, 6-thio-7-deaza-8-aza-guanosine, 7-methyl-guanosine (m⁷G), 6-thio-7-methyl-guanosine, 7-methyl-inosine, 6-methoxy-guanosine, 1-methyl-guanosine (m¹G), N2-methyl-guanosine (m²G), N2,N2-dimethyl-guanosine (m²₂G), N2,7-dimethyl-guanosine (m^{2,7}G), N2, N2,7-dimethyl-guanosine (m^{2,2,7}G), 8-oxo-guanosine, 7-methyl-8-oxo-guanosine, 1-methyl-6-thio-guanosine, N2-methyl-6-thio-guanosine, N2,N2-dimethyl-6-thio-guanosine, α-thio-guanosine, 2′-O-methyl-guanosine (Gm), N2-methyl-2′-O-methyl-guanosine (m²Gm), N2,N2-dimethyl-2′-O-methyl-guanosine (m²₂Gm), 1-methyl-2′-O-methyl-guanosine (m¹Gm), N2,7-dimethyl-2′-O-methyl-guanosine (m^{2,7}Gm), 2′-O-methyl-inosine (Im), 1,2′-O-dimethyl-inosine (m¹Im), and 2′-O-ribosylguanosine (phosphate) (Gr(p)).

In some embodiments, the nucleobase of the nucleotide is independently selected from a purine, a pyrimidine, a purine or pyrimidine analog. In some embodiments, the nucleobase and/or analog is each independently selected from adenine, cytosine, guanine, uracil, naturally-occurring and synthetic derivatives of a base, including but not limited to pyrazolo[3,4-d]pyrimidines, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-propynyl uracil and cytosine, 6-azo uracil, cytosine and thymine, 5-uracil (pseudouracil), 4-thiouracil, 8-halo (e.g., 8-bromo), 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, deazaguanine, 7-deazaguanine, 3-deazaguanine, deazaadenine, 7-deazaadenine, 3-deazaadenine, pyrazolo[3,4-d]pyrimidine, imidazo[1,5-a]1,3,5 triazinones, 9-deazapurines, imidazo[4,5-d]pyrazines, thiazolo[4,5-d]pyrimidines, pyrazin-2-ones, 1,2,4-triazine, pyridazine; and 1,3,5 triazine.

In some embodiments, the linear and/or circular polynucleotide comprises a nucleoside modification. In some embodiments, one or more atoms of a pyrimidine nucleobase is replaced or substituted, for example, with optionally substituted amino, optionally substituted thiol, optionally substituted alkyl (e.g., methyl or ethyl), optionally substituted or halo (e.g., chloro or fluoro) atoms or groups.

In some embodiments, uracil nucleosides of the linear and/or circular polynucleotide of the present disclosure are all modified. In some embodiments, the guanine nucleosides of the circular polynucleotide of the present disclosure are all modified. In some embodiments, the cytosine nucleosides of the linear and/or circular polynucleotide of the present disclosure are all modified. In some embodiments, the thymine nucleosides of linear and/or circular polynucleotide of the present disclosure are all modified. In some embodiments, the adenine nucleosides of linear and/or circular polynucleotide of the present disclosure are all modified. In some embodiments, the modifications to each nucleobase are the same. In some embodiments, the modifications to each nucleobase are different.

In some embodiments, modifications of the modified nucleosides and nucleotides are present in the sugar subunit. In some embodiments, the linear and/or circular polynucleotide (e.g., circRNAs) described herein comprise at least one sugar modification. Generally, RNA includes the sugar subunit: ribose, which is a 5-membered ring having an oxygen. In some embodiments, the 2′ hydroxyl group (OH) can be modified or replaced with a number of different substituents. Exemplary substitutions at the 2′OH-position include, but are not limited to, H, halo, optionally substituted C1-6 alkyl; optionally substituted C1-6 alkoxy; optionally substituted C6-10 aryloxy; optionally substituted C3-8 cycloalkyl; optionally substituted C3-8 cycloalkoxy; optionally substituted C6-10 aryloxy; optionally substituted C6-10 aryl-C1-6 alkoxy, optionally substituted C1-12 (heterocyclyl)oxy; a sugar (e.g., ribose, pentose, or any described herein); a polyethyleneglycol (PEG)-O(CH2CH2O)nCH2CH2OR, where R is H or optionally substituted alkyl, and n is an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20); and “locked” nucleic acids (LNA) in which the 2′-hydroxyl is connected by a C1-6 alkylene or C1-6 heteroalkylene bridge to the 4′-carbon of the same ribose sugar, where exemplary bridges include methylene, propylene, ether, or amino bridges; aminoalkyl; aminoalkoxy; amino; and amino acid.

Other exemplary sugar modifications include replacement of the oxygen (O) in ribose (e.g., with S, Se, or alkylene, such as methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for anhydrohexitol, altritol, mannitol, cyclohexanyl, cyclohexenyl, and morpholino that also has a phosphoramidate backbone); multicyclic forms (e.g., tricyclo; and “unlocked” forms, such as glycol nucleic acid (GNA) (e.g., R-GNA or S-GNA, where ribose is replaced by glycol units attached to phosphodiester bonds), threose nucleic acid (TNA, where ribose is replace with α-L-threofuranosyl-(3′->2′)), and peptide nucleic acid (PNA, where 2-amino-ethyl-glycine linkages replace the ribose and phosphodiester backbone).

In some embodiments, the sugar subunit contains one or more carbons that possess the opposite stereochemical configuration than that of the corresponding carbon in ribose. In some embodiments, polynucleotides as described herein, including circRNAs, include nucleotides containing, e.g., arabinose, as the sugar.

Nonlimiting examples of the sugar modification may include the modifications provided in Table 1. In some embodiments, the polynucleotides of the present invention have one or more nucleotides carrying a modification as provided in Table 1. In some embodiments, each of the nucleotides of a polynucleotide described herein carries any one of the modifications as provided in Table 1, or none of the modifications as provided in Table 1.

TABLE 1
Nucleotide Sugar Modifications

| Nucleotide | Structure Depiction |
|---|---|
| DNA | 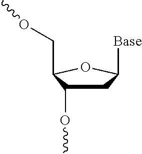 |
| 4′-C- aminomethyl- 2′-O-methyl RNA | 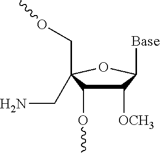 |
| 2′-O-Methyl (2′-OMe) | 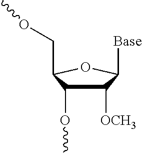 |
| 2′-azido | 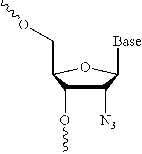 |
| 2′F-RNA | 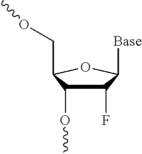 |
| Methylene- CLNA | 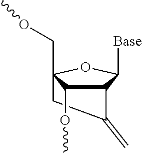 |
| 2′F-ANA | 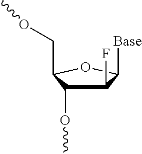 |
| N-MeO-amino BNA | 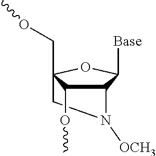 |
| 4′S-RNA | 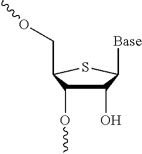 |
| N-Me- aminooxy BNA | 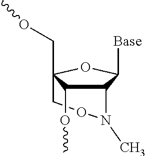 |
| UNA | 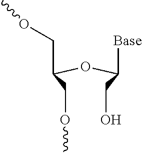 |
| 2′,4′- BNA^{NC}[NMe] | 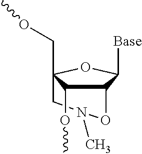 |
| LNA | 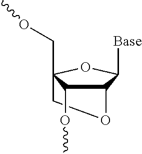 |
| MC | 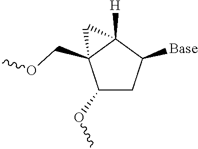 |
| 4′S-FANA | 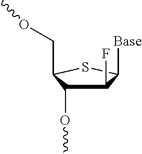 |
| ONA | 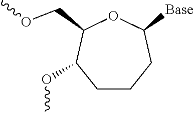 |
| 2′-O- Methoxyethyl (2′-MOE) | 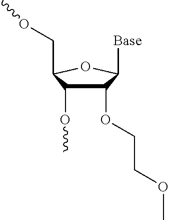 |
| tc-DNA | 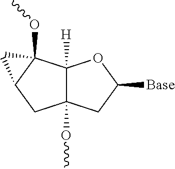 |
| 2′-O-Allyl | 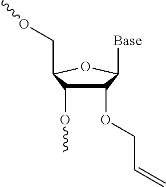 |
| CeNA | 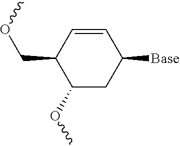 |
| 2′-O- Ethylamine | 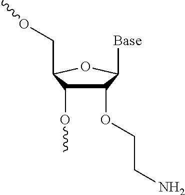 |
| ANA | 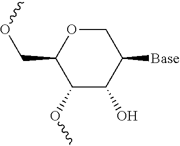 |
| 2′-O- Cyanoethyl | 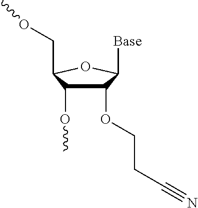 |
| HNA | 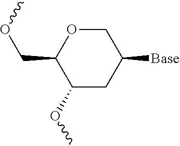 |
| 2′-O- Acetalester | 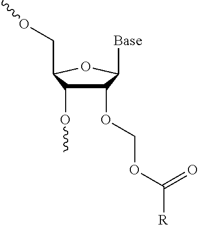 |

In some embodiments, at least one of the 2′ positions of the sugar (OH in RNA or H in DNA) of a nucleotide of the polynucleotides is substituted with —O— methoxyethyl, referred to as 2′-OMe. In some embodiments, at least one of the 2′ positions of the sugar (OH in RNA or H in DNA) of a nucleotide of the polynucleotides is substituted with —F, referred to as 2′-F. In some embodiments, the sugar modification is one or more locked nucleic acids (LNAs). In some embodiments, the polynucleotides are fully 2′-MOE-sugar modified.

In some embodiments, one or more modifications are present in the internucleoside linkage (the linking phosphate or the phosphodiester linkage or the phosphodiester backbone). In the context of the polynucleotide backbone, the phrases “phosphate” and “phosphodiester” are used interchangeably.

In some embodiments, backbone phosphate groups are modified by replacing one or more of the oxygen atoms with a different substituent. In some embodiments, modified nucleosides and nucleotides include replacement of an unmodified phosphate moiety with another internucleoside linkage as described herein. Examples of modified phosphate groups include, but are not limited to, phosphorothioate, methylphosphonates phosphoroselenates, boranophosphates, boranophosphate esters, hydrogen phosphonates, phosphoramidates, phosphorodiamidates, alkyl or aryl phosphonates, and phosphotriesters. Phosphorodithioates have both non-linking oxygens replaced by sulfur. The phosphate linker is also modified by the replacement of a linking oxygen with nitrogen (bridged phosphoramidates), sulfur (bridged phosphorothioates), and carbon (bridged methylene-phosphonates).

The α-thio substituted phosphate moiety is provided to confer stability to RNA and DNA polynucleotides through unnatural phosphorothioate backbone linkages. Phosphorothioate DNA and RNA have increased nuclease resistance and subsequently a longer half-life in a cellular environment. Phosphorothioate linked polynucleotide molecules are expected to also reduce the innate immune response through weaker binding/activation of cellular innate immune molecules.

In some embodiments, the linear and/or circular polynucleotides of the present disclosure comprise at least one phosphorothioate linkage, methylphosphonate linkage between nucleotides, 5′-(E)-vinylphosphonate (5′-E-VP), a phosphate mimic, as a modification.

In some embodiments, the internucleoside linkages of the polynucleotides may be partially or fully modified.

In some embodiments, modified nucleotides incorporated in the polynucleotides include, for example, 2′-O-Methyl-modified or 2′-O-Methoxyethyl-modified nucleotides (2′-OMe and 2′-MOE modifications, respectively), an alpha-thio-nucleoside (e.g., 5′-O-(1-thiophosphate)-adenosine, 5′-O-(1-thiophosphate)-cytidine (α-thio-cytidine), 5′-O-(1-thiophosphate)-guanosine, 5′-O-(1-thiophosphate)-uridine, or 5′-O-(1-thiophosphate)-pseudouridine.

Additional modifications to circular polynucleotides (e.g., circRNAs) of the present disclosure include, for example, modification or deletion of nucleotides (or codons) encoding one or more N-linked glycosylation site in a translated polypeptide.

In some embodiments, different sugar modifications, nucleobase modifications, and/or internucleoside linkages (e.g., backbone structures) are introduced at various positions in a polynucleotide described herein. One of ordinary skill in the art will appreciate that the nucleotide analogs or other modification(s) may be located at any position(s) of a polynucleotide such that the function of the polynucleotide is not substantially decreased.

In some embodiments, the one or more modified nucleotides is a 2′ O-methyl or a phosphorothioate modified nucleotide. Accordingly, in some embodiments, the one or more modified nucleotides comprises a 2′ O-methyl modification. In some embodiments, the one or more modified nucleotides comprises a phosphorothioate modification.

In some embodiments, the one or more modified nucleotides is selected from 2′-O-methyl 3′-phosphorothioate, 2′O-methyl, 2′-ribo 3′-phosphorothioate, 2′-fluro, 2′-O-methoxyethyl morpholino (PMO), locked nucleic acid (LNA), deoxy, or 5′ phosphate modified nucleotide. Accordingly, in some embodiments, the one or more modified nucleotides is a 2′-O-methyl 3′-phosphorothioate. In some embodiments, the one or more modified nucleotides is a 2′-O-methyl nucleotide. In some embodiments, the one or more modified nucleotides is a 2′-ribo 3′-phosphorothioate. In some embodiments, the one or more modified nucleotides is a 2′-fluro nucleotide. In some embodiments, the one or more modified nucleotides is a locked nucleic acid (LNA). In some embodiments, the one or more modifications comprises a 2′-O-methoxyethyl morpholino (PMO). In some embodiments, the one or more modifications comprises a deoxy modification. In some embodiments, the one or more modifications comprises a 5′ phosphate modification.

Various modified RNA bases are known in the art and include for example, 2′-O-methoxy-ethyl bases (2′-MOE) such as 2-MethoxyEthoxy A, 2-MethoxyEthoxy MeC, 2-MethoxyEthoxy G, 2-MethoxyEthoxy T. Other modified bases include for example, 2′-O-Methyl RNA bases, and fluoro bases. Various fluoro bases are known, and include for example, Fluoro C, Fluoro U, Fluoro A, Fluoro G bases. Various 2′OMethyl modifications can also be used with the methods described herein. For example, the following RNA comprising one or more of the following 2′OMethyl modifications can be used with the methods described: 2′-OMe-5-Methyl-rC, 2′-OMe-rT, 2′-OMe-rI, 2′-OMe-2-Amino-rA, Aminolinker-C6-rC, Aminolinker-C6-rU, 2′-OMe-5-Br-rU, 2′-OMe-5-I-rU, 2-OMe-7-Deaza-rG.

In some embodiments, the RNA comprises one or more of the following modifications: phosphorothioates, 2′O-methyls, 2′ fluoro (2′F), DNA. In some embodiments, the RNA comprises 2′OMe modifications at the 3′ and 5′-ends. In some embodiments, the RNA comprises one or more of the following modifications: 2′-O-2-Methoxyethyl (MOE), locked nucleic acids, bridged nucleic acids, unlocked nucleic acids, peptide nucleic acids, morpholino nucleic acids. In some embodiments, the RNA comprises one or more of the following base modifications: 2,6-diaminopurine, 2-aminopurine, pseudouracil, N1-methyl-psuedouracil, 5′ methyl cytosine, N6-methyladenosine, 2′pyrimidinone (zebularine), thymine. Other modified bases include for example, 2-Aminopurine, 5-Bromo dU, deoxyUridine, 2,6-Diaminopurine (2-Amino-dA), Dideoxy-C, deoxyInosine, Hydroxymethyl dC, Inverted dT, Iso-dG, Iso-dC, Inverted Dideoxy-T, 5-Methyl dC, 5-Methyl dC, 5-Nitroindole, Super T®, 2′-F-r(C,U), 2′-NH2-r(C,U), 2,2′-Anhydro-U, 3′-Desoxy-r(A,C,G,U), 3′-O-Methyl-r(A,C,G,U), rT, rI, 5-Methyl-rC, 2-Amino-rA, rSpacer (Abasic), 7-Deaza-rG, 7-Deaza-rA, 8-Oxo-rG, 5-Halogenated-rU, N-Alkylated-rN.

In some embodiments, other chemically modified RNA is used herein. For example, the RNA can comprise a modified base such as, for example, 5′, Int, 3′ Azide (NHS Ester); 5′ Hexynyl; 5′, Int, 3′ 5-Octadiynyl dU; 5′, Int Biotin (Azide); 5′, Int 6-FAM (Azide); and 5′, Int 5-TAMRA (Azide). Other examples of RNA nucleotide modifications that can be used with the methods described herein include for example phosphorylation modifications, such as 5′-phosphorylation and 3′-phosphorylation. The RNA can also have one or more of the following modifications: an amino modification, biotinylation, thiol modification, alkyne modifier, adenylation, Azide (NHS Ester), Cholesterol-TEG, and Digoxigenin (NHS Ester).

In some embodiments, the coding sequence within a circular RNA is modified. In one embodiment, up to 100% of the nucleosides of the coding polynucleotide sequence are modified. The modified nucleosides and nucleotides can include a modified nucleobase. Examples of nucleobases in RNA include, but are not limited to, adenine (A), guanine (G), cytosine (C), and uracil (U).

In one embodiment, at least one nucleoside modification is a uridine modification or an adenosine modification. In one embodiment, at least one nucleoside modification is selected from N6-methyladenosine (m6A), pseudouridine (ψ), N¹-methylpseudouridine (m1ψ), and 5-methoxyuridine (5moU). In one embodiment, the coding polynucleotide sequence is modified with methylpseudouridine (m1ψ). In one embodiment, the coding polynucleotide sequence is modified with 5-methoxyuridine (5moU).

In some embodiments, the coding sequence comprises 100% N6-methyladenosine (m6A) modification. In some embodiments, the coding sequence comprises at least 10%, at least 20%, at least 30%, at least 40%, at least 50%, at least 60%, at least 65%, at least 70%, at least 75%, at least 80%, at least 85%, at least 90%, at least 95%, or at least 99% N6-methyladenosine (m6A) modification.

In some embodiments, the coding sequence comprises 100% pseudouridine (ψ) modification. In some embodiments, the coding sequence comprises at least 10%, at least 20%, at least 30%, at least 40%, at least 50%, at least 60%, at least 65%, at least 70%, at least 75%, at least 80%, at least 85%, at least 90%, at least 95%, or at least 99% pseudouridine (ψ) modification. In some embodiments, the coding sequence comprises 100% N¹-methylpseudouridine (m1ψ) modification. In some embodiments, the coding sequence comprises at least 10%, at least 20%, at least 30%, at least 40%, at least 50%, at least 60%, at least 65%, at least 70%, at least 75%, at least 80%, at least 85%, at least 90%, at least 95%, or at least 99% N¹-methylpseudouridine (m1ψ) modification.

In some embodiments, the coding sequence comprises 100% 5-methoxyuridine (5moU) modification. In some embodiments, the coding sequence comprises at least 10%, at least 20%, at least 30%, at least 40%, at least 50%, at least 60%, at least 65%, at least 70%, at least 75%, at least 80%, at least 85%, at least 90%, at least 95%, or at least 99% 5-methoxyuridine (5moU) modification.

In some embodiments, the coding sequence is codon optimized. Codon optimization methods are known in the art and may be useful in efforts to achieve one or more of several goals. These goals include, but are not limited to, match codon frequencies in target and host organisms to ensure proper folding, alter GC content to increase mRNA stability or reduce secondary structures, minimize tandem repeat codons or base runs that may impair gene construction or expression, customize transcriptional and translational control regions, insert or remove protein trafficking sequences, remove/add post translation modification sites in encoded protein (e.g., glycosylation sites), add, remove or shuffle protein domains, insert or delete restriction sites, modify ribosome binding sites and RNA degradation sites, to adjust translational rates to allow the various domains of the protein to fold properly, or to reduce or eliminate problem secondary structures within the polynucleotide.

### Internal Ribosome Entry Site (IRES)

In some embodiments, a linear and/or circular RNA of the present invention comprises at least one internal ribosome entry site (IRES) element.

As used herein, the term “internal ribosome entry site” or “IRES” refers to an RNA sequence or structural element ranging in size from 10 nucleotides to 1,000 nucleotides or more which is capable of initiating translation of a polypeptide in the absence of a normal RNA cap structure. An IRES element may engage a eukaryotic ribosome for translation, or initiate cap-independent translation and protein synthesis. The internal ribosome entry site (IRES) allows translation to be initiated without an open 5′ capped end. The IRES can be selected from any class of IRES elements, i.e., any one of Types I-V. In some embodiments, the IRES element is a Type I IRES. In some embodiments, the IRES element is a Type II IRES. In some embodiments, the IRES element is a Type III IRES. In some embodiments, the IRES element is a Type IV IRES. In some embodiments, the IRES element is a Type V IRES.

In some embodiments, the IRES is derived from picornavirus cDNA, BiP-encoding DNA, Drosophila Antennapedia DNA, and/or bFGF-encoding DNA.

In some embodiments, the IRES is any one of encephalomyocarditis virus (EMCV) IRES, poliovirus IRES, Kaposi sarcoma-associated herpesvirus (KSHV) vFLIP IRES, or hepatitis C virus (HCV) IRES. In some embodiments, the IRES has a sequence of an IRES from Taura syndrome virus, Triatoma virus, Theiler's encephalomyelitis virus, Simian Virus 40, Solenopsis invicta virus 1, Rhopalosiphum padi virus, Reticuloendotheliosis virus, Human poliovirus 1, Plautia stali intestine virus, Kashmir bee virus, Human rhinovirus 2, Homalodisca coagulata virus 1, Human Immunodeficiency Virus type 1, Homalodisca coagulata virus 1, Himetobi P virus, Hepatitis C virus, Hepatitis A virus, Hepatitis GB virus, Foot and mouth disease virus, Human enterovirus 71, Equine rhinitis virus, Ectropis obliqua picorna-like virus, Encephalomyocarditis virus, Drosophila C Virus, Human coxsackievirus B3, Crucifer tobamovirus, Cricket paralysis virus, Bovine viral diarrhea virus 1, Black Queen Cell Virus, Aphid lethal paralysis virus, Avian encephalomyelitis virus, Acute bee paralysis virus, Hibiscus chlorotic ringspot virus, Classical swine fever virus, Human FGF2, Human SFTPAI, Human AMLI/RUNXI, Drosophila antennapedia, Human AQP4, Human ATIR, Human BAG-I, Human BCL2, Human BiP, Human c-IAPI, Human c-myc, Human eIF4G, Mouse NDST4L, Human LEFI, Mouse HIFI alpha, Human n.myc, Mouse Gtx, Human p27kipl, Human PDGF2/c-sis, Human p53, Human Pim-I, Mouse Rbm3, Drosophila reaper, Canine Scamper, Drosophila Ubx, Human UNR, Mouse UtrA, Human VEGF-A, Human XIAP, Drosophila hairless, S. cerevisiae TFIID, S. cerevisiae Y API, tobacco etch virus, turnip crinkle virus, EMCV-A, EMCV-B, EMCV-Bf, EMCV-Cf, EMCV pEC9, Picobirnavirus, HCV QC64, Human Cosavirus EID, Human Cosavirus F, Human Cosavirus JMY, Rhinovirus NAT00I, HRV14, HRV89, HRVC-02, HRV-A21, Salivirus A SHI, Salivirus FHB, Salivirus NG-JI, Human Parechovirus 1, Crohivirus B, Y c-3, Rosavirus M-7, Shanbavirus A, Pasivirus A, Pasivirus A 2, Echovirus El 4, Human Parechovirus 5, Aichi Virus, Hepatitis A Virus HA16, Phopivirus, CVAI0, Enterovirus C, Enterovirus D, Enterovirus J, Human Pegivirus 2, GBV-C GTI 10, GBV-C KI 737, GBV-C Iowa, Pegivirus A 1220, Pasivirus A 3, Sapelovirus, Rosavirus B, Bakunsa Virus, Tremovirus A, Swine Pasivirus 1, PLV-CHN, Pasivirus A, Sicinivirus, Hepacivirus K, Hepacivirus A, BVDVl, Border Disease Virus, BVDV2, CSFV-PK15C, SF573 Dicistrovirus, Hubei Picorna-like Virus, CRPV, Apodemus Agrarius Picornavirus, Caprine Kobuvirus, Parabovirus, Salivirus A BN5, Salivirus A BN2, Salivirus A 02394, Salivirus A GUT, Salivirus A CH, Salivirus A SZI, Salivirus FHB, CVB3, CVB1, Echovirus 7, CVB5, EVA71, CVA3, CVA12, EV24, o an aptamer to eIF4G, Coxsackievirus B3 (CVB3) or Coxsackievirus A (CVB1/2)

In some embodiments, the IRES is a variant of a wild type IRES sequence. An IRES variant may initiate translation in mammalian cells and requires minimal use of the cellular machinery for translation. The reduced reliance on the cellular machinery means that translation can be more efficient and can occur in the absence of a 5′-cap.

In some embodiments, the IRES is cricket paralysis virus IRES (CrPV-IRES) or Plautia stali intestine virus IRES (PSIV-IRES). Exemplary alternative IRES suitable for use in circular RNA include, but are not limited to, IRES from hepatitis C Virus (HCV), classical swine fever virus (CSFV), foot-and-mouth disease virus (FMDV), encephalomyocarditis virus (EMCV), polio virus, or hepatitis A virus.

In some embodiments, the circular RNA comprises an IRES that comprises a natural sequence. In some embodiments, the circular RNA comprises an IRES that comprises a synthetic sequence.

Different IRES elements (and exonic elements in general) affect the strength of protein expression as well as the cell/tissue specificity. Selection of an IRES element depends on the purpose of protein expression.

In some embodiments, the circular RNA comprises an IRES discussed in the PCT Patent Application Publications WO2021263124 and WO2022/271965; the contents of which are incorporated herein by reference in their entireties.

In some embodiments, the IRES sequence is about 10 to 1,000 nucleotides, or about 20 to 800 nucleotides, or about 50-600 nucleotides, or about 50-200 nucleotides in length. In some embodiments, the IRES element is at least about 5 nucleotides in length, at least about 8 nucleotides in length, at least about 9 nucleotides in length, at least about 10 nucleotides in length, at least about 15 nucleotides in length, at least about 20 nucleotides in length, at least about 25 nucleotides in length, at least about 30 nucleotides in length, or at least about 40 nucleotides in length. In some embodiments, the IRES sequence is about 50 nucleotides in length. In some embodiments, the IRES sequence is about 60 nucleotides in length. In some embodiments, the IRES sequence is about 70 nucleotides in length. In some embodiments, the IRES sequence is about 80 nucleotides in length. In some embodiments, the IRES sequence is about 90 nucleotides in length. In some embodiments, the IRES sequence is about 100 nucleotides in length. In some embodiments, the IRES sequence is about 150 nucleotides in length. In some embodiments, the IRES sequence is about 200 nucleotides in length. In some embodiments, the IRES sequence is about 250 nucleotides in length. In some embodiments, the IRES sequence is about 300 nucleotides in length. In some embodiments, the IRES sequence is about 350 nucleotides in length. In some embodiments, the IRES sequence is about 400 nucleotides in length. In some embodiments, the IRES sequence is about 500 nucleotides in length.

In one non-limiting example, the IRES is an IRES sequence from Coxsackievirus B3 (CVB3), or a variant thereof.

In some embodiments, the IRES element is operably linked to a coding sequence. In some embodiments, the IRES is operably linked to at least two coding sequences. In some embodiments, the circular RNA further includes a second IRES operably linked to a second coding sequence.

The IRES can increase the expression level of the protein of interest; preferably, the IRES element can increase the expression level of the protein of interest in eukaryotic cells.

In some embodiments, the IRES does not comprise a chemical modification. For example, the IRES may lack N1m-modification.

In some embodiments, the IRES element may be modified without impacting circular RNA functional features (e.g., translation efficiency, stability and reduced immunogenicity, etc.). The IRES element can comprise 1-20% modified nucleotides, or 1-10% modified nucleotides, or 5-20% modified nucleotides, or 5-10% modified nucleotides. In some embodiments, the IRES element comprises 1%, 2%, 3%, 4%, 5%, 6%, 7%, 8%, 9%, 10%, 11%, 12%, 13%, 14%, 15%, 16%, 17%, 18%, 19%, or 20% modified nucleotides. For example, the IRES element comprises 1%, 2%, 3%, 4%, 5%, 6%, 7%, 8%, 9%, or 10% modified nucleotides. As non-limiting examples, the IRES elements comprises 1%, 2%, 3%, 4%, 5%, 6%, 7%, 8%, 9%, or 10% N1-methyl-pseudouridine (N1m-ψ).

### Flanking Sequences-Untranslated Regions (UTRs)

In some embodiments, the linear and/or circular RNA comprises at least one untranslated region (UTR), such as a 5′ UTR and/or 3′ UTR. Untranslated regions (UTRs) of a gene are transcribed but not translated. The 5′ UTR starts at the transcription start site and continues to the start codon but does not include the start codon; whereas the 3′ UTR starts immediately following the stop codon and continues until the transcriptional termination signal. There is growing body of evidence about the regulatory roles played by the UTRs in terms of stability of the nucleic acid molecule and translation. The regulatory features of a UTR can be incorporated into the polynucleotides, primary constructs and/or mRNA of the present invention to enhance the stability of the molecule. The specific features can also be incorporated to ensure controlled down-regulation of the transcript in case they are misdirected to undesired organs sites.

In some embodiments, the linear and/or circular RNA comprises a 5′ UTR. The 5′ UTR is a natural 5′ UTR. Natural 5′ UTRs bear features which play roles in for translation initiation. They harbor signatures like Kozak sequences which are commonly known to be involved in the process by which the ribosome initiates translation of many genes. Kozak sequences have the consensus CCR (A/G) CCAUGG, where R is a purine (adenine or guanine) three bases upstream of the start codon (AUG), which is followed by another ‘G’, In some embodiments, the 5′ UTR is an engineered 5′ UTR. By engineering the features typically found in abundantly expressed genes of specific target organs, one can enhance the stability and protein production of the linear and/or circular RNA of the present disclosure. Other non-UTR sequences may be incorporated into the 5′ (or 3′ UTR) UTRs.

In some embodiments, the linear and/or circular RNA comprises a 5′ spacer. In some embodiments, the 5′ spacer sequence is about 10-100 nucleotides in length.

In some embodiments, the nucleic acid molecule provide herein comprises 3′ UTR. 3′ UTRs are known to have stretches of adenosines and uridines embedded in them. These AU rich signatures are particularly prevalent in genes with high rates of turnover. Based on their sequence features and functional properties, the AU rich elements (AREs) can be separated into three classes (Chen et al, 1995): Class I AREs contain several dispersed copies of an AUUUA motif within U-rich regions. C-Myc and MyoD contain class I AREs. Class II AREs possess two or more overlapping UUAUUUA (U/A) (U/A) nonamers. Molecules containing this type of AREs include GM-CSF and TNF-a. Class III ARES are less well defined. These U rich regions do not contain an AUUUA motif. Introduction, removal or modification of 3′ UTR AU rich elements (AREs) can be used to modulate the stability of the linear RNA and/or circular RNA of the present disclosure.

In some embodiments, the 3′ UTR may be derived from human beta-globin, human alpha-globin, xenopus beta-globin, xenopus alpha-globin. In other embodiments, the 3′ UTR is an engineered UTR sequence.

In some embodiments, one or more miRNA binding sites, and microRNA sequences may be included in the linear RNA and/circular RNA of the present disclosure. MicroRNAs (or miRNA) are 19-25 nucleotide long noncoding RNAs that bind to the 3′ UTR of nucleic acid molecules and down-regulate gene expression either by reducing nucleic acid molecule stability or by inhibiting translation. Such sequences may correspond to any known microRNA such as those taught in US Publication US2005/0261218 and US Publication US2005/0059005, the contents of which are incorporated herein by reference in their entireties.

Start Codon, Stop Codon, and polyA Tail

In some embodiments, the linear and/or circular RNA includes a region to initiate translation. In some embodiments, the region includes any translation initiation sequence or signal including a start codon. As a non-limiting example, the region includes a start codon. In some embodiments, the start codon is “ATG,” “ACG,” “AGG,” “ATA,” “ATT,” “CTG,” “GTG,” “TTG,” “AUG,” “AUA,” “AUU,” “CUG,” “GUG,” or “UUG”.

In some embodiments, the linear and/or circular RNA includes a region to stop translation. This region may include any translation termination sequence or signal including a stop codon. As a non-limiting example, the region includes a stop codon. In some embodiments, the stop codon may be “TGA,” “TAA,” “TGA,” “TAG,” “UGA,” “UAA,” “UGA” or “UAG.”

In some embodiments, the regions to initiate or terminate translation independently range from 3 to 40 nucleotides, e.g., at least 4, 5-30, 10-20, 10-15, or 4-30 nucleotides in length. Additionally, in some embodiments, these regions comprise, in addition to a start and/or stop codon, one or more signal and/or restriction sequences.

In some embodiments, a masking agent is used to mask a first start codon or alternative start codon in order to increase the chance that translation will initiate on a start codon or alternative start codon downstream to the masked start codon or alternative start codon. In some embodiments, the start codon is removed from the polynucleotide sequence in order to have the translation of the polynucleotide begin on a codon which is not the start codon. Translation of the polynucleotide begins on the codon following the removed start codon or on a downstream start codon or an alternative start codon. The polynucleotide sequence where the start codon is removed, in some embodiments, further comprises at least one masking agent for the downstream start codon and/or alternative start codons in order to control or attempt to control the initiation of translation, the length of the polynucleotide and/or the structure of the polynucleotide.

### RNA Sequence of Interest

In some embodiments, the linear and/or circular RNA does not encode a polypeptide or protein and functions as regulatory RNA. In some embodiments, the linear and/or circular RNA is a RNA therapeutic. In some embodiments, the linear and/or circular RNA is a RNA vaccine. In some embodiments, the RNA vaccine is an mRNA vaccine targeting cancer or infectious disease.

In some embodiments, linear and/or circular polynucleotides comprise a nucleic acid sequence element with a biological function, including but not limited to encoding a polypeptide of interest, a regulatory element of gene expression, a therapeutic nucleic acid molecule, a guide RNA, etc.

In some embodiments, the polypeptide may comprise two or more biological functions. Accordingly, linear and/or circular polynucleotides may be bifunctional. As the name implies, bifunctional circular polynucleotides, such as bifunctional circRNAs are those having or capable of at least two functions. In some embodiments, circular polynucleotides are multi-functional (e.g., have more than two functions).

In some embodiments, the linear and/or circular polynucleotides comprise a nucleic acid sequence element that encodes a polypeptide of interest. In some embodiments, the nucleic acid sequence element encodes two or more polypeptides. In some embodiments, the two or more polypeptides have the same amino acid sequences. In some embodiments, the two or more polypeptides have different amino acid sequences. In some embodiments, the polypeptide of interest may be one known in the art and/or described herein. In some embodiments, the circular polynucleotide acts as a messenger RNA (mRNA), having the same encoding function as its linear RNA counterpart.

The coding polynucleotide sequence can be unmodified, partially modified or completely modified. The modifications may be various distinct modifications. The modifications which render the nucleic acid molecules, when introduced to a cell, more resistant to degradation in the cell and/or more stable in the cell as compared to unmodified polynucleotides. The modifications may also increase the biological functions of nucleic acid molecules as compared to unmodified polynucleotides, such as binding to an RBP or another polynucleotide. The modifications may be structural and/or chemical modifications. The chemical modification may be a nucleotide and/or nucleoside modification including a nucleobase modification and/or a sugar modification, and a backbone linkage modification (i.e., the internucleoside linkage, e.g., a linking phosphate, a phosphodiester linkage, and a phosphodiester backbone). The structural modification may include a secondary structural modification, and a tertiary structural modification.

In one embodiment, the coding polynucleotide sequence contains at least one nucleoside modification. In some embodiments, circular RNA of the present disclosure may contain from about 1% to about 100% modified nucleotides (either in relation to overall nucleotide content, or in relation to one or more types of nucleotide, i.e. any one or more of A, G, T/U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 85% to 95%, from 85% to 100%, from 90% to 95%, from 90% to 100%, and from 95% to 100%).

In some embodiments, the polynucleotides are at least 10% modified in only one component of the nucleotide, with such component being the nucleobase, sugar, or linkage between nucleosides. For example, modifications may be made to at least 10%, 15%, 20%, 25%, 30%, 35%, 40%, 45%, 50%, 55%, 60%, 65%, 70%, 75%, 80%, 85%, 90%, 95% or 100% of the nucleobases, sugars, or linkages of a polynucleotide described herein.

In some embodiments, the polypeptide encompasses a plurality of polypeptides, full-length polypeptides and/or fragments of polypeptides, which are independently encoded by one or more nucleic acids, a plurality of nucleic acids, fragments of nucleic acids or variants of any of the aforementioned.

In some embodiments, the coding nucleic acid sequence element within the linear and/or circular polynucleotide is sufficient to encode a polypeptide of at least 10 amino acids in length, e.g., 10 to 5000 amino acids in length, or 10-1000 amino acids in length, or 50-2000 amino acids in length, or 30-3000 amino acids in length, or 100-1000 amino acids in length, or 100-3000 amino acids in length, or 200-1000 amino acids in length, or 200-500 amino acids in length, or 500-5000 amino acids in length, or 500-4000 amino acids in length, or 500-1500 amino acids in length, or 1000-5000 amino acids in length.

In some embodiments, the coding nucleic acid sequence element within the linear and/or circular polynucleotide may be greater than 30 nucleotides in length, e.g., at least or greater than about 35, 40, 45, 50, 55, 60, 70, 80, 90, 100, 120, 140, 160, 180, 200, 250, 300, 350, 400, 450, 500, 600, 700, 800, 900, 1000, 1100, 1200, 1300, 1400, 1500, 1600, 1700, 1800, 1900, 2,000, 2250, 2500, 3000, 3500, 4000, 4500, 5000, 5500, 6000, 6500, 7000, 7500, 8000, 8500, 9000, 9500, 10000, 20000, 30000, 40000, 50000, 60000, 70000, 80000, 90000, or 100000 nucleotides.

In some embodiments, the linear and/or circular polynucleotide of the present invention has a translation efficiency of at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, at least 2 fold, at least 3 fold, at least 4 fold, at least 5 fold, at least 6 fold, at least 7 fold, at least 8 fold, at least 9 fold, at least 10 fold, at least 15 fold, at least 20 fold, at least 25 fold, at least 30 fold, at least 35 fold, at least 40 fold, at least 45 fold, at least 50 fold, at least 55 fold, at least 60 fold, at least 65 fold, at least 70 fold, at least 75 fold, at least 80 fold, at least 85 fold, at least 90, at least 95 fold, or at least 100 fold greater than a linear counterpart.

In some embodiments, the encoded polypeptide is an antibody, a heavy chain of an antibody, a light chain of an antibody, a variable region of a heavy chain of an antibody, a variable region of a light chain of an antibody, a Fab fragment, and the like.

In some embodiments, the encoded polypeptide is an antigen. In some embodiments, the antigen is an antigen that causes infection such as viral antigen, a bacterial antigen. In some embodiments, the antigen is a cancer antigen such as a neoantigen and an antigen that is specific or associated with a cancer.

In some embodiments, the encoded antigen is a viral antigen. Once expressed in vivo, the antigen is recognized by the immune system to induce the desired immune response against the virus. In some embodiments, the virus is a respiratory virus, including but not limited to influenza, parainfluenza, rhinovirus, coronavirus (e.g., SARS-COV-2, MERS), Respiratory Syncytial Virus, parvovirus B19.

In some embodiments, the encoded polypeptide is a therapeutic protein or polypeptide. In some embodiments, the therapeutic protein is for example, an enzyme or a replacement therapy protein.

In some embodiments, the nucleic acid sequence element that encodes a polypeptide may be operatively linked to the IRES element of a circular polynucleotide. In some embodiments, the polypeptides of interest but are not limited to, biologics, cell penetrating peptides, secreted proteins, plasma membrane proteins, cytoplasmic or cytoskeletal proteins, intracellular membrane bound proteins, nuclear proteins, proteins associated with human disease, targeting moieties or any other protein encoded by the human genome.

In some embodiments, the linear and/or circular RNA may encode additional features which facilitate trafficking of the polypeptides to therapeutically relevant sites. One such feature which aids in protein trafficking is the signal sequence. As used herein, a “signal sequence” or “signal peptide” is a polynucleotide or polypeptide, respectively, which is from about 9 to 200 nucleotides (3-60 amino acids) in length which is incorporated at the 5′ (or N-terminus) of the coding region or polypeptide encoded, respectively. Addition of these sequences result in trafficking of the encoded polypeptide to the endoplasmic reticulum through one or more secretory pathways. Some signal peptides are cleaved from the protein by signal peptidase after the proteins are transported.

In some embodiments, the coding nucleic acid sequences are codon optimized.

### Codon Optimization

The linear and/or circular polynucleotides of the present invention such as circRNAs, their regions or parts or subregions may be codon optimized. In some embodiments, the coding sequences of the linear and/or circular polynucleotides are codon optimized. Codon optimization methods are known in the art and may be useful in efforts to achieve one or more of several goals. These goals include, but are not limited to, match codon frequencies in target and host organisms to ensure proper folding, alter GC content to increase mRNA stability or reduce secondary structures, minimize tandem repeat codons or base runs that may impair gene construction or expression, customize transcriptional and translational control regions, insert or remove protein trafficking sequences, remove/add post translation modification sites in encoded protein (e.g. glycosylation sites), add, remove or shuffle protein domains, insert or delete restriction sites, modify ribosome binding sites and mRNA degradation sites, to adjust translational rates to allow the various domains of the protein to fold properly, or to reduce or eliminate problem secondary structures within the polynucleotide.

In some embodiments, a codon optimized sequence may be one in which codons in a polynucleotide encoding a polypeptide have been substituted in order to increase the expression, stability and/or activity of the polypeptide. Factors that influence codon optimization include, but are not limited to one or more of: (i) variation of codon biases between two or more organisms or genes or synthetically constructed bias tables, (ii) variation in the degree of codon bias within an organism, gene, or set of genes, (iii) systematic variation of codons including context, (iv) variation of codons according to their decoding tRNAs, (v) variation of codons according to GC %, either overall or in one position of the triplet, (vi) variation in degree of similarity to a reference sequence for example a naturally occurring sequence, (vii) variation in the codon frequency cutoff, (viii) structural properties of mRNAs transcribed from the DNA sequence, (ix) prior knowledge about the function of the DNA sequences upon which design of the codon substitution set is to be based, and/or (x) systematic variation of codon sets for each amino acid.

In some embodiments, a codon optimized polynucleotide may minimize ribozyme collisions and/or limit structural interference between the expression sequence and the IRES.

Codon optimization tools, algorithms and services are known in the art, non-limiting examples include, but are not limited to, software from GeneArt (Life Technologies), DNA2.0 (Menlo Park Calif.) and/or proprietary methods.

In some embodiments, the ORF sequence is optimized using optimization algorithms. Codon options for each amino acid are given in Table 2.

TABLE 2
Codon Options

| Single | | |
|---|---|---|
| Letter | | |
| Nomen- | Amino Acid | |
| clature | Name | Codon Options |
| A | Alanine | GCT, GCC, GCA, GCG |
| C | Cysteine | TGT, TGC |
| D | Aspartic acid | GAT, GAC |
| E | Glutamic acid | GAA, GAG |
| F | Phenylalanine | TTT, TTC |
| G | Glycine | GGT, GGC, GGA, GGG |
| H | Histidine | CAT, CAC |
| I | Isoleucine | ATT, ATC, ATA |
| K | Lysine | AAA, AAG |
| L | Leucine | CTT, CTC, CTA, CTG, TTA, TTG |
| M | Methionine | ATG |
| N | Asparagine | AAT, AAC |
| P | Proline | CCT, CCC, CCA, CCG |
| Q | Glutamine | CAA, CAG |
| R | Arginine | CGT, CGC, CGA, CGG, AGA, AGG |
| s | Serine | TCT, TCC, TCA, TCG, AGT, AGC |
| Sec | Selenocysteine | UGA in mRNA in presence of |
| | | Selenocysteine insertion element |
| | | (SECTS) |
| Stop | Stop codons | TAA, TAG, TGA |
| T | Threonine | ACT, ACC, AC A, ACG |
| V | Valine | GTT, GTC, GTA, GTG |
| w | Tryptophan | TGG |
| γ | Tyrosine | TAT, TAC |

### Signaling Nucleotides

In some embodiments, the linear and/or circular polynucleotides encode additional for example, a signal sequence which facilitates the trafficking of the polypeptides to therapeutically relevant sites.

As described herein, the linear and/or circular polynucleotides of the present disclosure comprise regions that are partially or substantially not translatable, e.g., having a noncoding region. Such noncoding regions are different from the non-coding functional sequences and are located in any region of the linear and/or circular polynucleotide. The non-coding regions include but are not limited to the linker, the spacer and/or the flanking regions. In some embodiments, the noncoding regions are located in more than one region of the linear and/or circular polynucleotide.

### Non-Coding Functional Sequences

In some embodiments, the linear and/or circular polynucleotide of the present invention comprises a nucleic acid sequence element that has a regulatory function, i.e., the sequence is a non-coding sequence but has a biological function and/or activity (e.g., a non-coding function). The non-coding functions of the linear and/or circular RNA includes but is not limited to interactions with other types of non-coding RNA molecules, microRNAs, long noncoding RNAs, and RNA-binding proteins. In some embodiments, the linear and/or circular polynucleotide encodes a guide RNA used in CRISPR gene editing.

In some embodiments, the linear and/or circular polynucleotide of the present invention comprises a nucleic acid sequence acting as a miRNA sponge, which competes with endogenous RNA. In some embodiments, the miRNA sponge sequence comprise at least one miRNA response element (MRE) that binds to a miRNA and negatively regulates its activity. In some embodiments, circular polynucleotides such as circular RNAs described herein down-regulate miRNA activity and/or up-regulate the expression of miRNA target genes.

In some embodiments, the miRNA sponge sequence may comprise 1-150 conserved miRNA target sites to bind to a miRNA and to sponge miRNA, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 95, 100, or 150 miRNA target sites to sponge the miRNA.

In some embodiments, the linear and/or circular polynucleotide described herein may regulate more than one miRNA, e.g., 2, 3, 4, 5, 6, 7, 8, 9, 10 or more miRNAs.

In some embodiments, the linear and/or circular polynucleotide comprises a nucleic acid sequence that binds to one or more RNA binding proteins (RBPs) acting as a protein sponge.

In some embodiments, the linear and/or circular polynucleotide comprises a nucleic acid sequence that interacts with one or more protein to enhance protein function.

In some embodiments, the linear and/or circular polynucleotide comprises a nucleic acid sequence that acts as scaffold to mediate complex formation between specific enzymes and substrates.

In some embodiments, the linear and/or circular polynucleotides comprises a nucleic acid sequence that binds to one or more protein to recruit proteins to specific locations.

In some embodiments, the linear and/or circular polynucleotide comprises one or more long noncoding RNA (lncRNA, or lincRNA), a small nucleolar RNA (sno-RNA), microRNA (miRNA), small interfering RNA (siRNA) or Piwi-interacting RNA (piRNA) and/or a portion thereof.

In some embodiments, the functional non-coding sequences are included in the linear and/or circular polynucleotide alone and used for their functions. In some embodiments, the functional non-coding sequences are included in the circular polynucleotide encoding a polypeptide of interest.

Genome Manipulation Using gRNA

In some embodiments, circular guide RNA is used with a suitable gene editing system, e.g., CRISPR-Cas for targeted gene editing leading to increasing or decreasing the expression level of a desired target gene. In some embodiments, the circular gRNA is used for targeted transcription activation, targeted transcription repression, targeted epigenome modification, or targeted genome modification through specific interactions between gRNA and CRISPR-Cas and a target sequence.

In some embodiments, the gene editing protein is capable of binding to the RNA guide and causing a break in the target nucleic acid sequence complementary to the RNA guide, and/or editing the target nucleic acid sequence complementary to the RNA guide.

In some embodiments, the gene editing method or system comprises a fusion protein with an effector that modifies target DNA in a site-specific manner, where the modifying activity includes methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, demyristoylation activity, integrase activity, transposase activity, recombinase activity, polymerase activity, ligase activity, helicase activity, or nuclease activity, any of which can modify DNA or a DNA-associated polypeptide (e.g., a histone or DNA binding protein).

In some embodiments, the gene editing method or system comprises a fusion protein with enzymes that can edit DNA sequences by chemically modifying nucleotide bases, including deaminase enzymes that can modify adenosine or cytosine bases and function as site-specific base editors.

In some embodiments, the synthetic circular guide RNA described herein is used in a gene editing method or system to modulate transcription of target DNA, or target non-coding RNA, including tRNA, rRNA, snoRNA, siRNA, miRNA, and long ncRNA.

In some embodiments, the synthetic circular guide RNA is used for targeted engineering of chromatin loop structures using a suitable gene editing system. Targeted engineering of chromatin loops between regulatory genomic regions provides a means to manipulate endogenous chromatin structures and enable the formation of new enhancer-promoter connections to overcome genetic deficiencies or inhibit aberrant enhancer-promoter connections.

In some embodiments, the synthetic circular guide RNA is used in conjunction with a gene editing system for correction of pathogenic mutations by insertion of beneficial clinical variants or suppressor mutations.

### Delivery of RNA Therapeutics

### Lipid Based Carriers

In some embodiments, RNA based drug delivery is carried out through lipid nanoparticle (LNP) delivery.

In some embodiments, LNPs are used to target specific cells using endogenous or exogenous ligands by encapsulating siRNA, mRNA, and circular RNA by methods known in the art. Endocytosis of LNPs destabilizes the endosomal membrane and release linear and/or circular RNAs or siRNAs into the cytoplasm.

In general, LNPs can be characterized as small solid or semi-solid particles possessing an exterior lipid layer with a hydrophilic exterior surface that is exposed to the non-LNP environment, an interior space which may aqueous (vesicle like) or non-aqueous (micelle like), and at least one hydrophobic inter-membrane space. LNP membranes may be lamellar or non-lamellar and may be comprised of 1, 2, 3, 4, 5 or more layers.

The LNPs for formulating the circular polynucleotides of the present disclosure may have a diameter from 10-1000 nm. The nanoparticle may be 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, or 1000 nm, or greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm. In some embodiments, a LNP may have a diameter from about 1 to about 100 nm, such as but not limited to, from about 1 nm to about 10 nm, about 1 nm to about 20 nm, from about 1 nm to about 30 nm, from about 1 nm to about 40 nm, from about 1 nm to about 50 nm, from about 1 nm to about 60 nm, from about 1 nm to about 70 nm, from about 1 nm to about 80 nm, from about 1 nm to about 90 nm, from about 5 nm to about from 100 nm, from about 5 nm to about 10 nm, about 5 nm to about 20 nm, from about 5 nm to about 30 nm, from about 5 nm to about 40 nm, from about 5 nm to about 50 nm, from about 5 nm to about 60 nm, from about 5 nm to about 70 nm, from about 5 nm to about 80 nm, from about 5 nm to about 90 nm, and/or from about 5 nm to about 100 nm.

LNPs useful herein are known in the art and generally comprise cholesterol (aids in stability and promotes membrane fusion), a helper lipid (e.g., a phospholipid which provides structure to the LNP bilayer and also may aid in endosomal escape), a polyethylene glycol (PEG) derivative (which reduces LNP aggregation and “shields” the LNP from non-specific endocytosis by immune cells and reduce opsonization by serum proteins and reticuloendothelial clearance), and an ionizable lipid (complexes negatively charged RNA and enhances endosomal escape), which form the LNP-forming composition. The components of the LNP may be selected based on the desired target, tropism, cargo (e.g., a circular polynucleotide), size, or other desired feature or property. The relative amounts (ratio) of ionizable lipid, helper lipid, cholesterol and PEG substantially affect the efficacy of lipid nanoparticles and may be optimized for a given application and administration route.

The LNP for formulating the circular polynucleotides of the present disclosure comprises at least one cationic lipid. The cationic lipid may be selected from any lipid known in the art such as, but not limited to, DLin-MC3-DMA, DLin-DMA, C12-200 and DLin-KC2-DMA. The LNP for formulating the circular polynucleotides of the present disclosure may comprise at least one helper lipid. The helper lipids in LNPs may contribute to their stability and delivery efficiency, and/or mitigate the toxicity owing to the cationic lipids. In some embodiments, the helper lipid is a lipid having cone-shape geometry, e.g., dioleoylphosphatidylethanolamine (DOPE). In some embodiments, the helper lipid is a cylindrical-shaped lipid such as phosphatidylcholine.

The LNP for formulating the circular polynucleotides of the present disclosure comprises a cholesterol, a naturally occurring cholesterol analogue, or a synthetic cholesterol like compound and the cholesterol derivatives. In some embodiments, a naturally occurring cholesterol analog may be selected from those by Patel et al., (Nature Communications, 2020; 983: doi.org/10.1038/s41467-020-14527-2); the contents of which are incorporated herein by reference in their entirety. In some embodiments, the LNPs comprise one or more cholesterol derivatives, e.g., PtdChol.

The LNP for formulating the circular polynucleotides of the present disclosure comprises at least a PEGylated compound, such as a PEG polymer and a PEGylated lipid.

In some embodiments, the circular polynucleotides (circRNAs) and circRNA compositions of the present disclosure may be formulated using one or more lipids and/or lipidoids. As used herein, the term “lipidoid” refers to any material having characteristics of a lipid. Lipidoids can be lipid-like structures containing multiple secondary and tertiary amine functionalities, which confer highly efficient interaction with nucleic acid molecules.

The synthesis of lipids and lipidoids has been extensively discussed and formulations containing the lipids and lipidoids are particularly suitable for delivery of nucleic acids. Use of the lipids and lipidoids to formulate and effectively deliver double stranded small RNAs (siRNAs), singled stranded mRNAs and gene therapy has been described in mice and non-human primates (e.g., Levins et al., 2010); Akinc et al., Nat Biotechnol. 2008 26:561-569; Love et al., Proc Natl Acad Sci USA. 2010, 107:1864-1869; Siegwart et al., Proc Natl Acad Sci USA. 2011, 108:12996-3001; Leuschner et al., Nat Biotechnol. 2011, 29:1005-1010; Roberts et al., Methods Mol. Biol., 2016, 1364:2991-310; Ball et al. Nato. Lett. 2018, 18 (6): 3814-3822; Lokras et al., Methods Mol. Biol., 2021, 2282:137-157; Schrom et al., Mol. Ther. Nucleic Acids, 2017, 7:350-365; the contents of all of which are incorporated herein by references in their entirety.)

The lipids and lipidoids can be cationic lipids and lipidoids. Cationic lipids typically features a positively charged head group followed by hydrophobic tails of varying compositions, wherein the head and tail are connected by a linker, such as an ether, ester or amide. Without wishing to be bound by any theory, their cationic head groups neutralize the anionic charges of the nucleic acids that they transport.

In some embodiments, ionizable cationic lipids can be used for formulations. Exemplary ionizable lipids such as Dlin-MC3-DMA (MC3), Dlin-KC2-DMA (KC2), and cKK-E12 may be used for package circular nucleic acid molecules.

In some embodiments, the lipids can be anionic lipids. In other embodiments, the lipids and lipidoids can be neutral lipids. In some embodiments, the LNP comprise a cholesterol lipid.

In some embodiments, the circular polynucleotides (e.g., circRNAs) compositions of the present disclosure may be formulated using one or more polymers, or polymer containing nanoparticles (NPs). In some embodiments, the polymer may be biocompatible and biodegradable.

The physicochemical properties of polymers (e.g., composition, molecular weight, and polydispersity) can be modified to achieve specialized formulations for nucleic acid delivery. Polymers may be naturally derived or synthetic. In some embodiments, the polymers used in the present disclosure have undergone processing to reduce and/or inhibit the attachment of unwanted substances such as, but not limited to, bacteria, to the surface of the polymer. The polymer may be processed by methods known and/or described in the art and/or described in PCT Patent Application Publication No. WO2012150467; the contents of which are herein incorporated by reference in their entirety.

In some embodiments, the circular polynucleotides of the present disclosure may be formulated using naturally derived polymers, structural proteins and polysaccharides, such as cationic collagen derivatives and chitosan. Cationic collagenous proteins have been used for nucleic acid delivery to articular cartilage and bone for regenerative medicine and metastatic tumor treatment (Capito et al., Gene Ther., 2007, 14:721-732; Curtin et al., Adv. Healthc. Mater., 2015, 4:223-227). Chitosan, a linear cationic polysaccharide, is produced by the deacetylation of chitin (poly-d-glucosamine), which is non-toxic even at a high concentration and can be formulated into polyplexes. A non-limiting example of chitosan-based formulation includes a core of positively charged chitosan and an outer portion of negatively charged substrate (U.S. Pub. No. US20120258176; the contents of which are herein incorporated by reference in their entirety). Chitosan includes, but is not limited to N-trimethyl chitosan, mono-N-carboxymethyl chitosan (MCC), N-palmitoyl chitosan (NPCS), EDTA-chitosan, low molecular weight chitosan, chitosan derivatives, or combinations thereof.

The circular polynucleotides of the present disclosure may be formulated using synthetic polymers which may incorporate versatile chemistries in a controlled manner providing flexibility and more options for polynucleotide formulations. Various synthetic strategies exist in the art to control polymerization reactions and, therefore, the properties of the resulting polymer. Examples of methods include controlled free-radical polymerizations such as reversible addition-fragmentation chain transfer (RAFT) polymerization and atom transfer radical polymerization (ATRP) (Boyer et al., Chem. Rev., 2009, 109:5402-5436). The polymers formulated with the circular polynucleotide compositions of the present disclosure may be synthesized by the methods described in PCT Patent Application Publication Nos. WO2012082574 or WO2012068187; the contents of each of which are herein incorporated by reference in their entirety.

In some embodiments, the circular polynucleotide compositions of the present disclosure may comprise at least one polymeric compound such as but not limited to, polyethenes, polyethylene glycol (PEG), poly(l-lysine) (PLL), PEG grafted to PLL, cationic lipopolymer, biodegradable cationic lipopolymer, polyethyleneimine (PEI), cross-linked branched poly(alkylene imines), a polyamine derivative, a modified poloxamer, a biodegradable polymer, elastic biodegradable polymer, biodegradable block copolymer, biodegradable random copolymer, biodegradable polyester copolymer, biodegradable polyester block copolymer, biodegradable polyester block random copolymer, multiblock copolymers, linear biodegradable copolymer, poly[α-(4-aminobutyl)-L-glycolic acid) (PAGA), biodegradable cross-linked cationic multi-block copolymers, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester), acrylic polymers, amine-containing polymers, dextran polymers, dextran polymer derivatives or combinations thereof.

The synthetic polymers are biodegradable. Synthetic biodegradable polymers may be generated by assembling low molecular weight monomers into polymers via bioreversible linkages such as sulfide or ester bonds. Examples of synthetic biodegradable polymers include, but are not limited to, poly(lactic acid) (PLA), poly(glycolic acid) (PGA), poly(lactic-co-glycolic acid) (PLGA), poly(beta amino) esters (PBAEs), Poly(amine-co-esters) (PACEs).

Biodegradable polymers have been previously used to protect nucleic acids from degradation and been shown to result in sustained release of payloads in vivo (Rozema et al., Proc Natl Acad Sci USA. 2007, 104:12982-12887; Sullivan et al., Expert Opin Drug Deliv. 2010, 7:1433-1446; Convertine et al., Biomacromolecules. 2010 October 1; Chu et al., Acc Chem Res. 2012 January 13; Manganiello et al., Biomaterials. 2012, 33:2301-2309; Benoit et al., Biomacromolecules. 2011, 12:2708-2714; Singha et al., Nucleic Acid Ther. 2011, 2:133-147; de Fougerolles Hum Gene Ther. 2008, 19:125-132; Schaffert and Wagner, Gene Ther. 2008, 16:1131-1138; Chaturvedi et al., Expert Opin Drug Deliv. 2011, 8:1455-1468; Davis, Mol Pharm. 2009, 6:659-668; Davis, Nature, 2010, 464:1067-1070; the contents of each of which are herein incorporated by reference in their entirety.

The biodegradable polymers may be polymers comprising a polyethylenimine group as described in U.S. Pat. No. 7,700,542. The polymers may be the biodegradable cationic lipopolymer made by methods described in U.S. Pat. No. 6,696,038, and U.S. Pub. Nos. US20030073619 and US20040142474; the contents of each of which are incorporated herein by reference in their entirety.

The circular polynucleotides and circular polynucleotide compositions of the disclosure can be formulated using one or more liposomes. As used herein, the term “liposome” refers to an artificially prepared vesicle which may primarily be composed of one or several lipid bilayers and may be used as a delivery vehicle. Liposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 and 500 nm in diameter.

Liposomes may be lipid-based liposomes, polymer-based liposomes, or hybrids. Liposomes can be cationic liposomes, neutral liposomes. Cationic liposomes have been used to deliver siRNA to various cell types (e.g., US Patent Application Publication No.: US2004/0204377).

The compositions of the present disclosure can be delivered using one or more lipoplexes.

In some embodiments, gold nanoparticles are used to deliver linear and/or circular RNAs due to their high stability, purity, and easy surface modification.

### Engineered Exosomes

Exosomes are tiny vesicles smaller than 50 nm secreted by mature reticulocytes, which are associated with transferrin receptors and function in antigen presentation during the regulation of immune cells. In some embodiments, engineered exosomes act as cargo carriers and deliver small hydrophilic or lipophilic molecules, including some therapeutic drugs to cells, participating in the regulation of many major diseases. Exosomes can improve bioavailability of some drugs when taken orally, reducing the total dose required for administration, and minimizing side effects.

### Viral Like Particles (VLPs)

In some embodiments, RNA therapeutics discussed herein are delivered using viral delivery particles. Viral particles include recombinant viruses and viral like particles (VLPs). As used herein, the term Virus-like particles (VLPs) are molecules that closely resemble viruses, but are non-infectious because they contain no viral genetic material. They can be naturally occurring or synthesized through the individual expression of viral structural proteins, which can then self-assemble into the virus-like structure. Combinations of structural capsid proteins from different viruses can be used to create recombinant VLPs. VLPs can be produced from different viruses, such as adeno-associated viruses, retroviruses, lentiviruses and vesiculoviruses. VLPs can be produced in multiple cell culture systems including bacteria, mammalian cell lines, insect cell lines, yeast and plant cells. VLPs possess diverse applications in therapeutics, immunization, and diagnostics. VLPs have been synthesized in a wide range of ESs, including prokaryotic (bacteria) and eukaryotic (insect cells, mammalian cell lines, plant cells, or yeast). The functionality of VLPs can be increased through modifying their exterior or interior surface by displaying the heterologous epitopes of interest using different methods like peptide conjugation, genetic fusion, and chemical crosslinking.

In some embodiments, the VLP is derived from a Vesiculovirus. In some embodiments, the VLP is derived from VSV (Indiana vesiculovirus, formerly Vesicular stomatitis Indiana virus (VSIV or VSV). In some embodiments, the virus like particle comprises a mutated VSV-G protein. VSV-G protein is a single transmembrane glycoprotein (G) which plays a critical role during the initial steps of virus infection. it is responsible for virus attachment to specific receptor, LDL-R. In the cell, G protein triggers the fusion between the viral and endosomal membranes, which releases the viral genome in the cytosol for the subsequent steps of infection. In some embodiments, VSV-G protein is mutated to abolish its binding to LDL-R receptor. For example, a VSV-G envelope protein may be a mutated at one or more of any one of H8, K47, Y209, and/or R354. In some embodiments, a VLP may comprise a mutated VSV-G protein described in the PCT patent application Publication No. WO2019057974; the contents of which are incorporated herein by reference in their entireties. In some aspects, the VLP for delivery RNA therapeutics is a viral particle disclosed in the PCT Publication Nos. WO2020236263 and WO2023107886; the contents of each of which are incorporated herein by reference in their entireties.

In some embodiments, the virus like particle is pseudotyped. As a non-limiting example, the virus like particle is VSV-G-pseudotyped lentiviruses (VSV-G-LVs).

In some embodiments, the viral particle for delivering RNA therapeutics is a retrovirus, a recombinant AAV, or an adenovirus.

### Pharmaceutical Compositions

In one aspect of the present disclosure, the linear and/or circular RNA described herein may be provided in compositions, e.g., pharmaceutical compositions.

In some embodiments, the present disclosure provides compositions, e.g., compositions comprising a linear and/or circular RNA and a pharmaceutically acceptable carrier. In one aspect, the present disclosure provides pharmaceutical compositions comprising an effective amount of a circular RNA described herein and one or more pharmaceutically or physiologically acceptable carriers, excipients or diluents. In some embodiments, pharmaceutical compositions of the present disclosure may comprise a circular RNA expressing cell, e.g., a plurality of circular RNA-expressing cells, as described herein, in combination with one or more pharmaceutically or physiologically acceptable carriers, excipients or diluents.

In some embodiments, a pharmaceutically acceptable carrier can be an ingredient in a pharmaceutical composition, other than an active ingredient, which is nontoxic to the subject.

A pharmaceutically acceptable carrier can include, but is not limited to, a buffer, excipient, stabilizer, or preservative. Examples of pharmaceutically acceptable carriers are solvents, dispersion media, coatings, antibacterial and antifungal agents, isotonic and absorption delaying agents, and the like that are physiologically compatible, such as salts, buffers, saccharides, antioxidants, aqueous or non-aqueous carriers, preservatives, wetting agents, surfactants or emulsifying agents, or combinations thereof. The amounts of pharmaceutically acceptable carrier(s) in the pharmaceutical compositions may be determined experimentally based on the activities of the carrier(s) and the desired characteristics of the formulation, such as stability and/or minimal oxidation.

In some embodiments, the pharmaceutically acceptable carrier include buffers such as acetic acid, citric acid, histidine, boric acid, formic acid, succinic acid, phosphoric acid, carbonic acid, malic acid, aspartic acid, Tris buffers, HEPPSO, HEPES, neutral buffered saline, phosphate buffered saline and the like; carbohydrates such as glucose, sucrose, mannose, or dextrans, mannitol; proteins; polypeptides or amino acids such as glycine; antioxidants; chelating agents such as EDTA or glutathione; adjuvants (e.g., aluminum hydroxide); antibacterial and antifungal agents; and preservatives.

Pharmaceutical compositions of the present disclosure can be formulated for a variety of means of parenteral or non-parenteral administration. In one embodiment, the compositions can be formulated for infusion or intravenous administration. Compositions disclosed herein can be provided, for example, as sterile liquid preparations, e.g., isotonic aqueous solutions, emulsions, suspensions, dispersions, or viscous compositions, which may be buffered to a desirable pH. Formulations suitable for oral administration can include liquid solutions, capsules, sachets, tablets, lozenges, and troches, powders liquid suspensions in an appropriate liquid and emulsions.

In some embodiments, circular RNAs of the present disclosure are encapsulated in or complexed with lipids, lipidoids, liposomes, lipid nanoparticles (LNPs), polymer based nanoparticles, natural and synthetically-derived exosome.

In some embodiments, a composition may comprise about 0.001 μg to 100 μg of circular RNA described herein. In some embodiments, a composition may comprise about 0.001 μg, 0.01 μg, 0.1 μg, 1.0 μg, 1.5 μg, 2.0 μg, 2.5 μg, 3.0 μg, 3.5 μg, 4.0 μg, 4.5 μg, 5.0 μg, 6.0 μg, 7.0 μg, 8.0 μg, 9.0 μg, 10.0 μg, 15.0 μg, 20.0 μg, 25.0 μg, 30 μg, 40 μg, 50 μg, 60 μg, 70 μg, 80 μg, 80 μg, 100 μg or more circular RNA described herein.

### Methods of Treatment

In another aspect, the present invention provides methods of uses of linear and/or circular RNAs, and compositions comprising RNA molecules of the present invention.

In some embodiments, the present disclosure is directed to a method of expressing a target protein in a cell, said method comprising transfecting a circular RNA encoding the target protein into the cell. In one embodiment, the method comprises transfecting using lipofection or electroporation. In another embodiment, the circular RNA is transfected into a cell using a nanocarrier. In yet another embodiment, the nanocarrier is a lipid, polymer or a lipo-polymeric hybrid.

In some embodiments, the cell is an in vitro cell such as a cell line, or an in vivo cell. In some embodiments, the cell is a eukaryotic cell.

In one embodiment, cells can transiently express the circular RNA described herein for 1, 2, 3, 4, 5, 6, 7, 8, 9, 10, 11, 12, 13, 14, 15 days or more after introduction.

Transient expression of the circular RNA can be affected by the method of delivery. In one embodiment, the circular RNA is transduced into the cell by electroporation. In one embodiment, the circular RNA is introduced into the cell by lipid transfection methods known in the art.

In one embodiment, the present disclosure provides a method of treating a disease in a subject with a circular RNA described herein that encodes a therapeutic protein. Accordingly, the method comprises administering a therapeutically effective amount of a composition comprising a circular RNA described herein to a subject for the treatment of a subject having, or at risk of developing, a disease or disorder, e.g., cancer. In another embodiment, the disclosure relates to administering a therapeutically effective amount of a composition comprising a circular RNA described herein for the treatment of a subject having a disease involving loss of a functional gene.

Any suitable route of administration can be used. In certain embodiments, the administration of the compositions may be carried out in any manner, e.g., by parenteral or nonparenteral administration, including by aerosol inhalation, injection, infusions, ingestion, transfusion, implantation or transplantation. For example, the compositions described herein may be administered to a patient trans-arterially, intradermally, subcutaneously, intratumorally, intramedullary, intranodally, intramuscularly, by intravenous (i.v.) injection, intranasally, intrathecally or intraperitoneally. In one aspect, the compositions of the present disclosure are administered intravenously. In one aspect, the compositions of the present disclosure are administered to a subject by intradermal or subcutaneous injection. The compositions may be injected, for instance, directly into a tumor, lymph node, tissue, organ, or site of infection.

In some embodiments, a circular RNA as described herein may be used in combination with other known agents and therapies. Administered “in combination”, as used herein, means that two (or more) different treatments are delivered to the subject during the course of the subject's treatment e.g., the two or more treatments are delivered after the subject has been diagnosed with the disease and before the disease has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In some embodiments, circular RNA molecules described herein are administered to an individual to stimulate an immune response. The immune response can comprise a humoral immune response, a cell-mediated immune response, or both. Cellular immune response relates typically to the activation of macrophages, natural killer cells (NK), Helper T-cell (Tₕ) response, antigen-specific cytotoxic T-lymphocytes, and the release of various cytokines in response to an antigen. In more general terms, cellular immune response is based on the activation of cells of the immune system. Typically, a cellular immune response may be characterized e.g., by activating antigen-specific cytotoxic T-lymphocytes that are able to induce apoptosis in cells, e.g., specific immune cells like dendritic cells or other cells, displaying epitopes of foreign antigens on their surface. Such cells may be virus-infected or infected with intracellular bacteria, or cancer cells displaying antibodies. Further characteristics may be activation of macrophages and natural killer cells, enabling them to destroy pathogens and stimulation of cells to secrete a variety of cytokines that influence the function of other cells involved in adaptive immune responses and innate immune responses. Humoral immune response refers typically to antibody production and optionally to accessory processes accompanying antibody production. A humoral immune response may be typically characterized, e.g., by Th2 activation and cytokine production, germinal center formation and isotype switching, affinity maturation and memory cell generation. Humoral immunity also typically may refer to the effector functions of antibodies, which include pathogen and toxin neutralization, classical complement activation, and opsonin promotion of phagocytosis and pathogen elimination.

A cell-mediated immune response can comprise, a Helper T-cell (Tₕ) response, a CD8+ cytotoxic T-cell (CTL) response, or both. In some embodiments the immune response comprises a humoral immune response, and the antibodies are neutralizing antibodies.

Pharmaceutical compositions of the present disclosure may be administered in a manner appropriate to the disease to be treated (or prevented). The quantity and frequency of administration will be determined by such factors as the condition of the subject, and the type and severity of the subject's disease, although appropriate dosages may be determined by clinical trials.

### Kits

The present disclosure provides a kit comprising one or more circular RNA molecules described herein.

In some embodiments, the present disclosure provides a kit comprising agents for making a circular RNA. The agents include, but are not limited to, agents for synthesizing liner RNA fragments, one or more DNA splint fragments and/or one or more RNA ligases.

All publications, patent applications, patents, and other references mentioned herein are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

### EXAMPLES

Various aspects of the invention are described in further detail in the following examples. The following examples describe some of the preferred modes of making and practicing the present invention. However, it should be understood that these examples are for illustrative purposes only and are not meant to limit the scope of the invention.

### Example 1: Generation of Circular RNA by Ligation

This example illustrates RNA circularization using DNA splint and an exemplary single strand (ss) RNA ligase, T4 RNA ligase 1. As illustrated in FIGS. 1A and 1B, a linear precursor RNA (labeled as “linear RNA”) was annealed with a DNA “gap” splint (FIG. 1A) or a DNA no-“gap” splint (FIG. 1B) comprising regions complementary to the 5′ and 3′ ends of the linear RNA. The splint secured the two ends of linear RNA in place to be ligated by a ssRNA ligase, for example, T4 RNA ligase I. Following subsequent DNase treatment to remove the splint, a circular RNA was formed.

Generation of RNA with a 5′ Monophosphate

A linear RNA sequence was synthesized in vitro, for example, using in vitro transcription (IVT). Plasmid containing an upstream T7 promoter followed by a desired RNA sequence (e.g., 5′ UTR—IRES—coding sequence—3′ UTR) was cut by restriction enzyme digest to produce a template with a free 3′ end for in vitro transcription. The DNA template was mixed with T7 polymerase, T7 buffer and free nucleotides (ATP, CTP, GTP, UTP) to allow for template-mediated transcription in vitro to produce a desired RNA molecule. After in vitro transcription, the reaction was treated with DNase to remove the template DNA. The RNA from the IVT was treated with Antarctic phosphatase (AnaP) to remove the 5′ triphosphate (i.e., dephosphorylate to 5′-OH), and then was treated with poly nucleotide kinase (PNK) to put a monophosphate on the 5′ end to generate a 5′-monoP end.

Alternatively, a 5× excess of GMP (relative to GTP) was included in the in vitro transcription reaction mixture. GMP can only incorporate at the 5′ start site, so this allowed for generation of an RNA with a 5′ monophosphate.

RNA from the IVT was then purified by silica column clean up.

### Splint Mediated Ligation of Circular RNAs

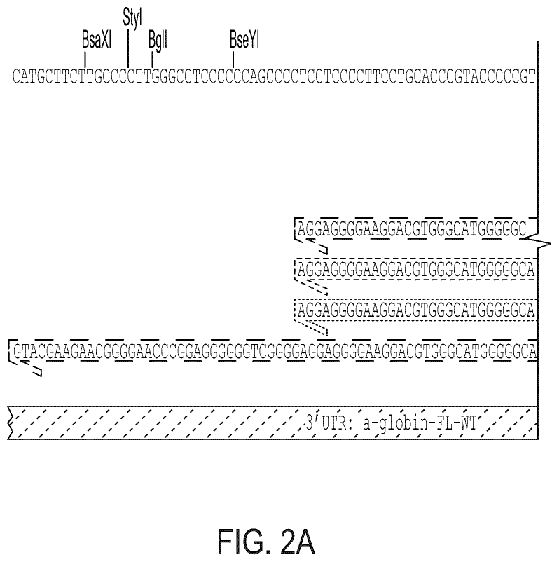

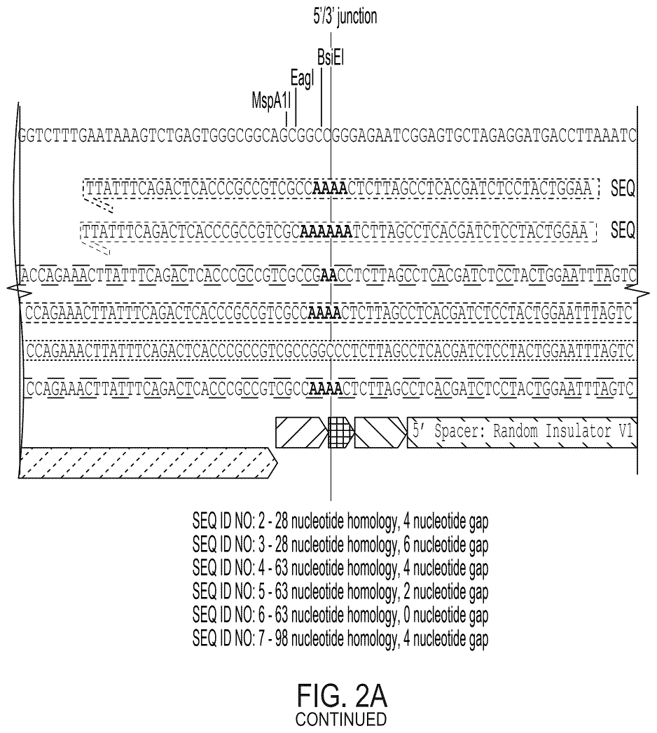

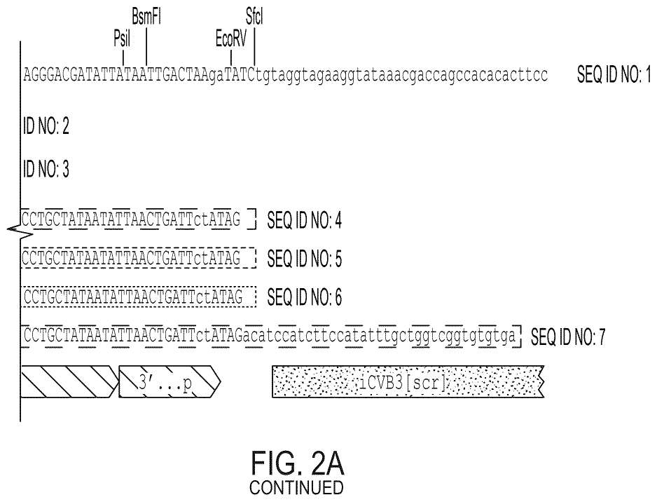

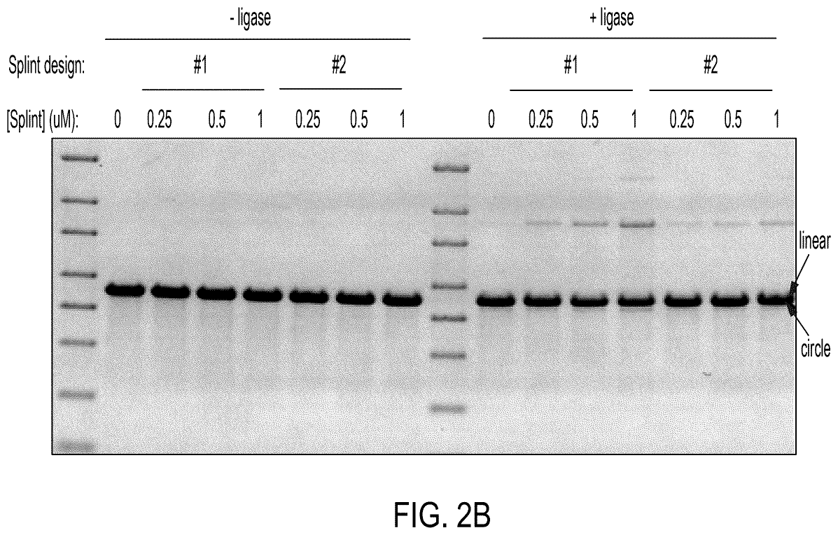

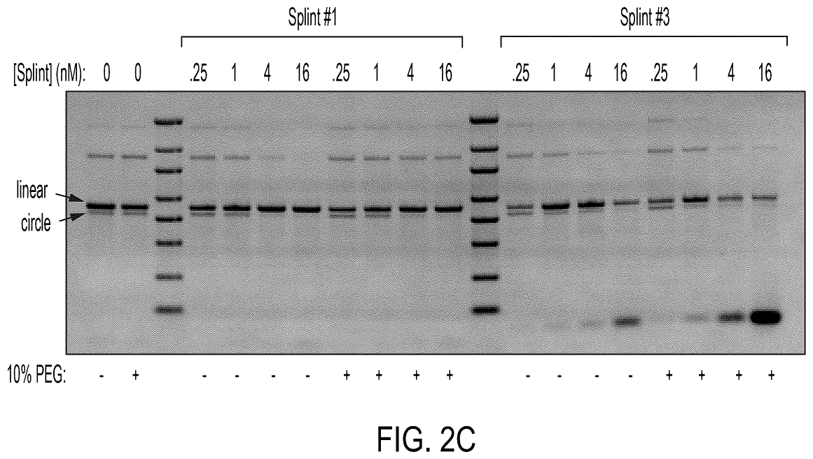

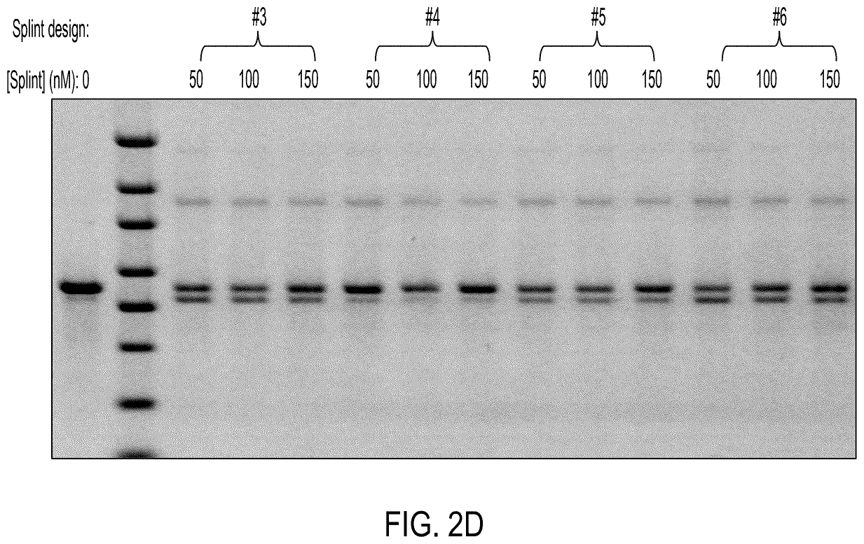

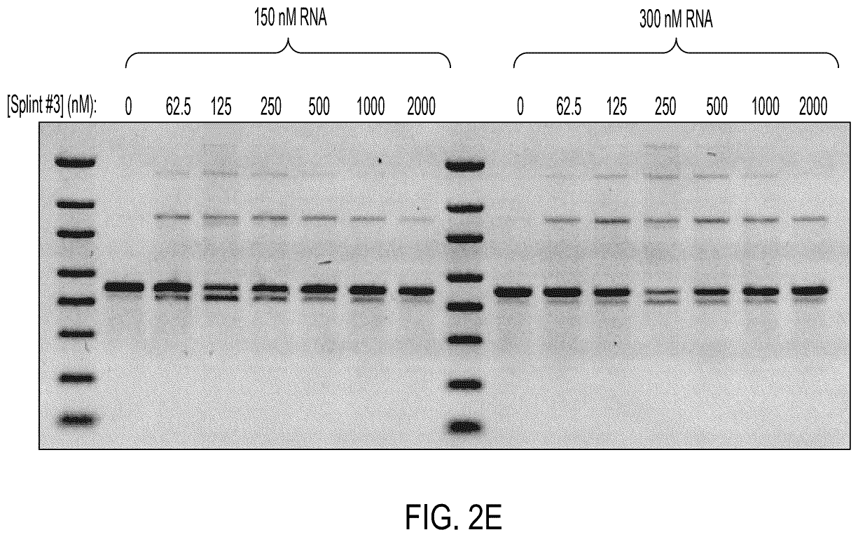

In vitro transcribed linear RNA with a 5′ monophosphate (5′monoP) was mixed with indicated splints (FIG. 2A) at specified concentrations, heated up to 75° C. for 3 minutes and slowly cooled to 25° C. (cooling rate of ~0.1° C. per second). This heat/cool step was performed in refolding buffer (50 mM Tris pH 7, 0.5 mM EDTA, 50 mM NaCl). This allowed for heat denaturation of RNA and splint annealing to desired sites on RNA. The RNA concentration was kept relatively low, generally below 500 nM. This low concentration favored circularization and decreased the amount of inter-molecular ligations (between two RNA molecules). The results indicated that a 1:1 molar ratio of RNA to splint was optimal for ligation. After the splint annealing step, T4 RNA ligase buffer was added (50 mM Tris pH 7.5, 10 mM MgCl2, 1 mM DTT, 100 μM ATP, 0.02 U/μl SuperaseIn), followed by addition of 1-1.5 U/ul T4 RNA ligase I. The ligase reaction was incubated at 25° C. for 2 hours. The RNA was concentrated by centrifugal filtration and purified by silica column clean up.

### Optimization of Splints

Splint design for optimal RNA circularization was extensively explored. As shown in FIG. 2A, splint fragments with different homology sizes and gaps were designed. The optimal splint design was a “gap” splint with over 33 nucleotides of homology to both the 5′ and 3′ ends of the RNA with a spacer of four ‘A’ nucleotides in the direct middle of the splint. That is, splints contained over 33 nucleotides of homology to the 5′ end, followed by two As that were specifically designed to not be complementary to the 5′ end and instead leave the terminal two nucleotides on the 5′ end of the RNA “unpaired.” The same design was followed for the 3′ end, with over 33 nucleotides of homology to the 3′ end with the final two nucleotides left intentionally unpaired by replacing the complementary nucleotides in the splint with two A nucleotides. The splint brings the 5′ and 3′ ends into proximity, but leaves the terminal two nucleotides on either end un-base paired. The unpaired overhangs create an ideal substrate for T4 RNA ligase I, which is a single stranded RNA ligase. In general, any “gap” in the splint or any splint design that resulted in one or more unpaired nucleotides (even without a “gap” in the splint) at the 5′ or the 3′ end resulted in some level of circularization.

As shown in FIG. 1B, an alternative splint design includes no gap between the two complementary regions that hybridize to the 5′ and 3′ ends of the RNA. The gapless splint after binding to the RNA creates overhangs at the 5′ end and 3′ end of the RNA, forming a single stranded region for ligase I.

### Purification of Ligated Circular RNAs

After ligation, the splint was removed by adding excess of another splint that was exactly complimentary to the ‘gap’ splint used for ligation. The reaction was then heated to 75° C. for 3 minutes and slowly cooled to 25° C. (cooling rate of ~0.1° C. per second) in refolding buffer (50 mM Tris pH 7, 0.5 mM EDTA, 50 mM NaCl). The mixture was then incubated with DNase to remove the splint. Ligated circular RNAs were then further purified by reverse phase HPLC to enrich for the ligated circular RNA over the un-ligated RNA.

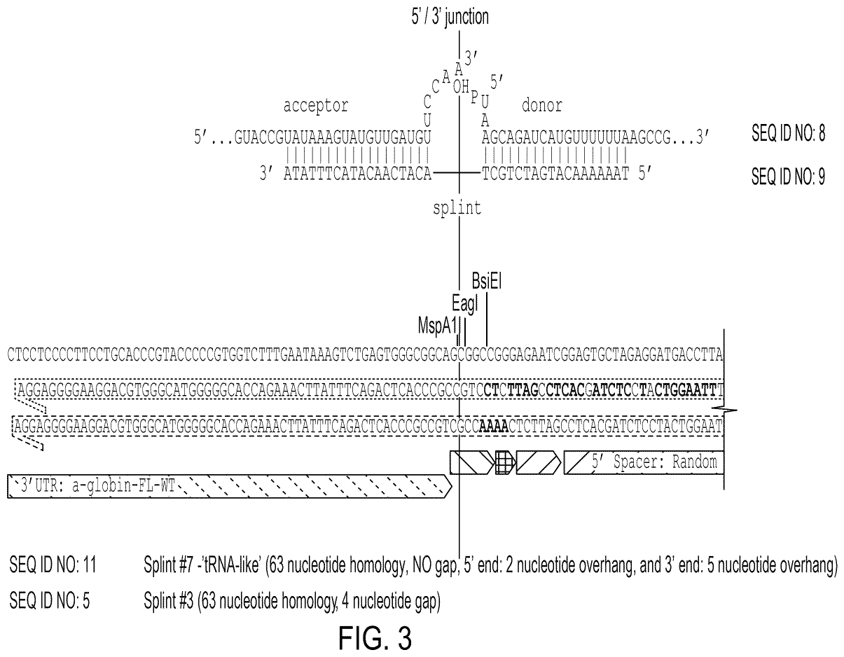

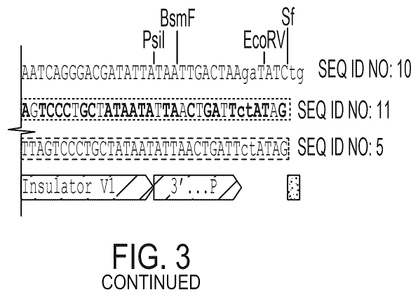

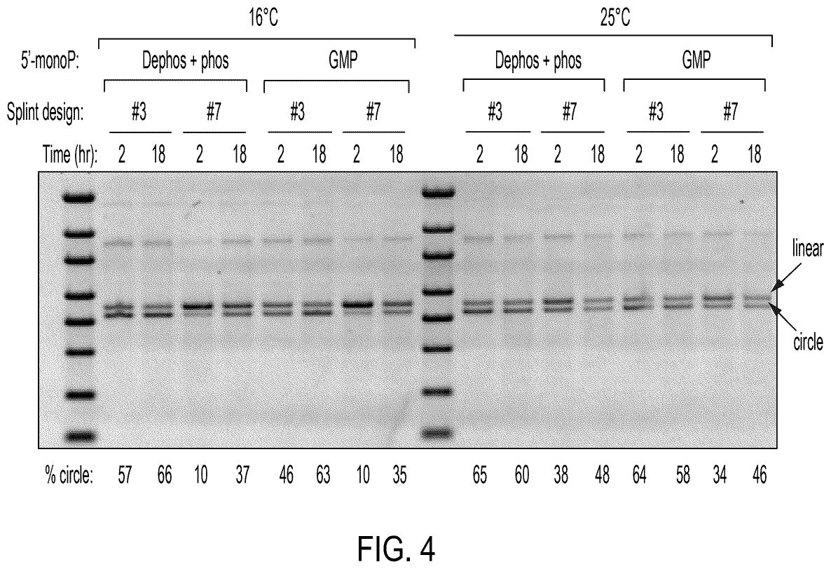

The Results as shown in FIGS. 2-4 indicated that the design using a DNA splint with a gap of 4 nucleotides had higher percentage of circularization. The results also showed that the DNA splint with a longer complementary region on each side had greater circularization, e.g., Splint #3.

### Example 2: Generation of Circular RNA with 100% N1m-Ψ and 5Mo-U Modifications

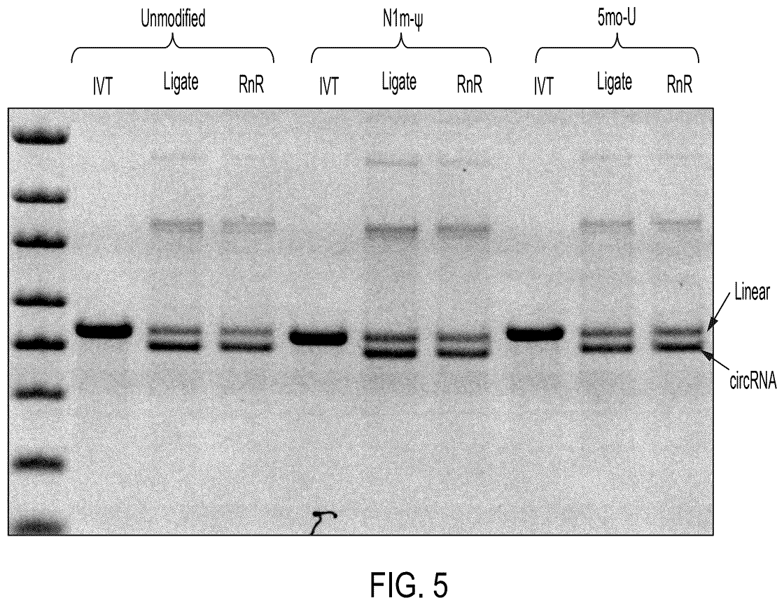

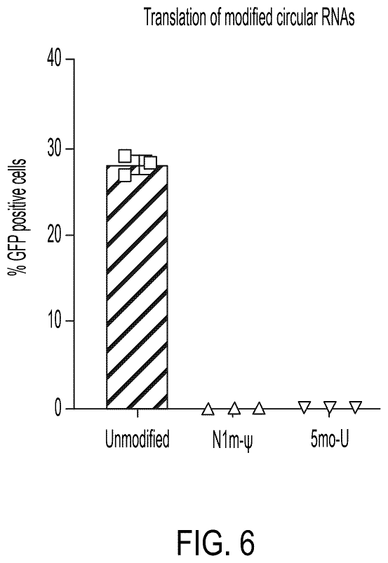

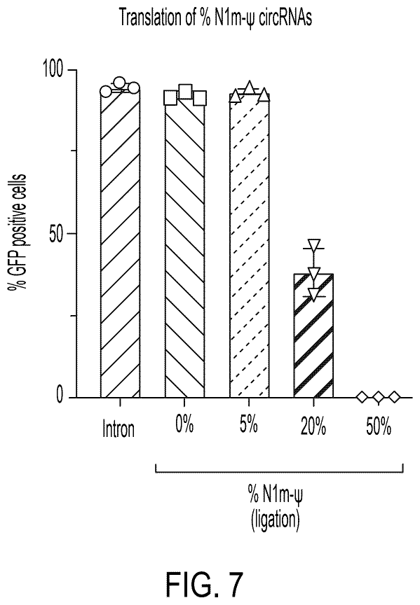

Linear RNA precursors comprising 100% N1m-Ψ or 5mo-U modifications were transcribed in vitro. The RNA encodes a Green Fluorescent Protein (GFP) reporter. The modified linear RNA was mixed with a DNA splint and a T4 RNA ligase 1 and incubated. The ligated circular RNA molecules were purified, and denaturing gel electrophoresis was performed. The gel image (FIG. 5) confirmed that the ligation method generated circular RNA with 100% N1m-Y′ or 5mo-U modifications. The circular RNA molecules were transfected into HEK293 cells and analyzed for GFP fluorescence by FACS analysis 24 hours after transfection. An unmodified circular RNA comprising the same sequence was generated using the same method and was transfected into cells as control. As shown in FIG. 6, unmodified circular RNAs generated by ligation were translated in cells (unmodified lane), while circular RNAs generated by ligation with 100% N1m-Ψ or 5mo-U modifications were not translated in cells. Circular RNAs having different percentage of modifications, e.g., 5%, 20% and 50% were ligated and transfected into cells for expression. As shown in FIG. 7, circular RNAs generated by ligation with less than 20% N1m-Ψ ere translated efficiently in cells, while circular RNAs generated by ligation with more than 20% N1m-Ψ ere not translated efficiently in cells.

Circular RNAs generated using the ligation method described in Example 1 and circular RNAs comprising the same sequence that were generated using T4 phage td gene intron mediated self-splicing were tested and compared. Circular RNAs generated by ligation were comparable in translatability relative to circular RNAs made by intron-mediated splicing (compare intron to 0% lanes in FIG. 7).

### Example 3: Generation of Chimeric Circular RNA from Two or More Linear RNA Fragments

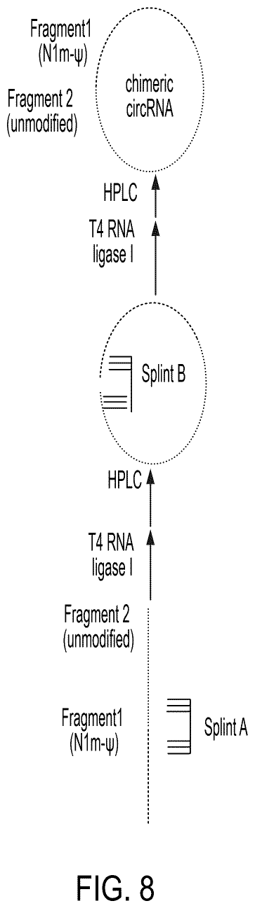

This example illustrates a two-step ligation process to circularize two linear RNA fragments. FIG. 8 illustrates the process.

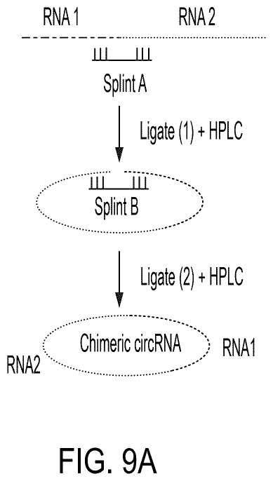

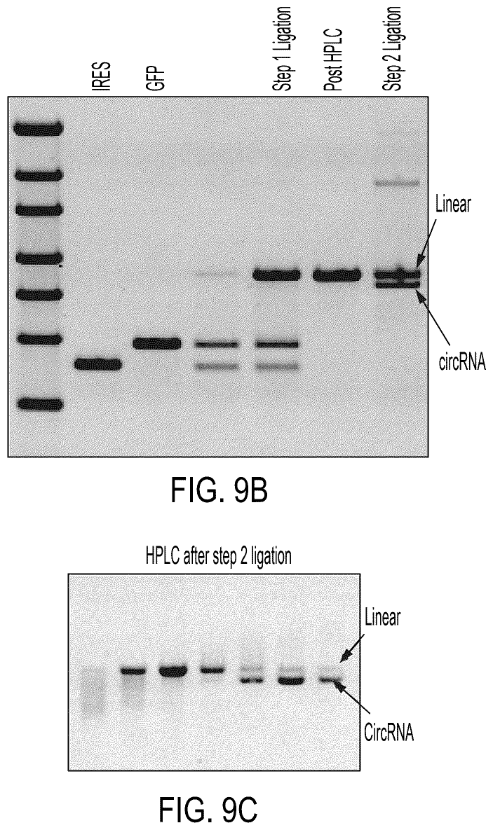

In one experiment, a separate and unmodified IRES and a 100% N1m-ψ modified GFP coding sequence were in vitro transcribed. The IRES and the coding sequence were ligated to form a linear RNA molecule using a first DNA splint with a RNA ligase. The IRES-coding sequence that encodes a GFP reporter was purified using reverse phase HPLC and circularized using a second DNA splint with an RNA ligase as described in Example 1 (FIG. 9A). As shown in FIG. 9B-9C, chimeric circular RNAs can be generated using the two-step process shown in FIG. 9A. The results showed that circular RNA could be made by multistep ligation using two or more splints and ligation steps.

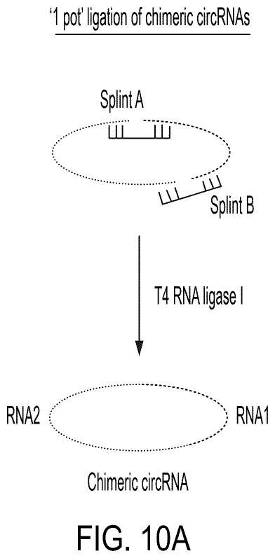

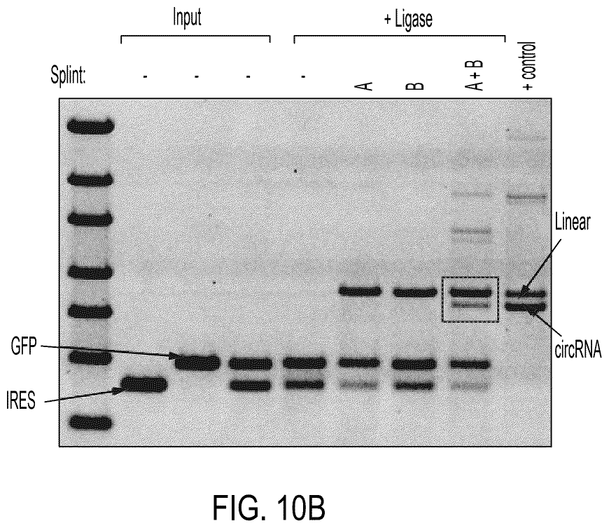

In another experiment, a separate unmodified IRES and a 100% N1m-Ψ modified GFP coding sequence were in vitro transcribed. The IRES, GFP coding sequence, 2 splints, and RNA ligase were mixed in one reaction (FIG. 10A). The circular RNA was purified from other un-ligated products using HPLC. As shown in the gel image of FIG. 10B, circular RNA was made by one-step ligation using two or more splints in one reaction.

### Example 4: Concentrating the Annealed DNA Splint: RNA Before Ligation

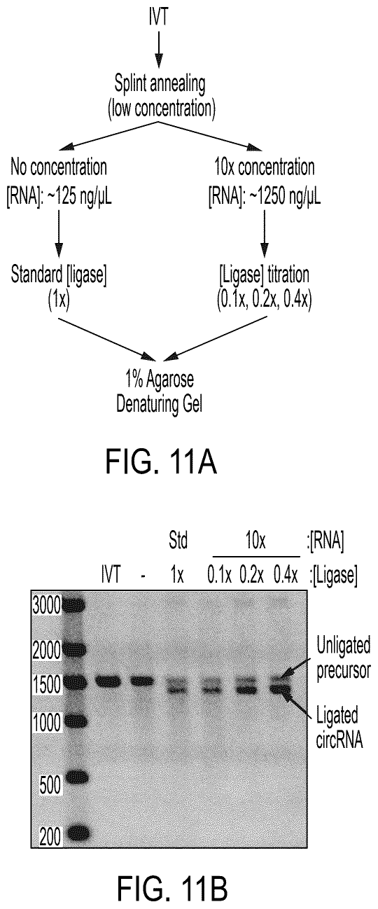

As discussed in Example 1, to minimize the inter-molecular ligations, the RNA concentration was kept low during the ligation. This example demonstrated that after annealing the DNA splint and RNA at a low concentration, the mixture was concentrated before ligation using ligase. As shown in FIG. 11A, following splint annealing, the reaction was concentrated up to about 1250 ng/μl RNA; three different concentrations of RNA ligase I (0.1×, 0.2×, 0.4× the amount of total ligase used relative to the standard (1× ligase) method without concentration (for example, 0.1× indicates 10% of ligase was used) were used and compared for ligation efficiency. As shown in FIG. 11B, the ligation efficiency was increased significantly (resulting in more than 75% RNA circularization).

### Example 5: Removing the Splint with Reverse Splint

This example tested the use of reverse splint after the splinted ligation reaction to remove un-ligated RNA.

After incubating with ligase I, 10× concertation of the reverse splint (relative to the concentration of the splint) was added to the reaction. The mixture was heated to remove the DNA splint from the RNA and bind to the reverse splint. As described herein, the reverse splint comprises a sequence complementary to the splint and a poly(A) sequence at either 5′ end or 3′ end. The splint:reverse splint duplex was then treated with DNase. The ligated RNA was effectively isolated after removing the splint using Reverse Phase HPLC or SEC. The splint:reverse splint duplex was also removed by oligo dT purification, whereby the poly(A) sequence on the reverse splint was bound to the oligo dT resin. In contrast, circRNA did not bind the column and was then isolated.

### Example 6: Polyadenylating Un-Ligated RNA by polyA Polymerase

This example tested polyadenylation of un-ligated RNA after the splinted ligation reaction to remove un-ligated RNA.

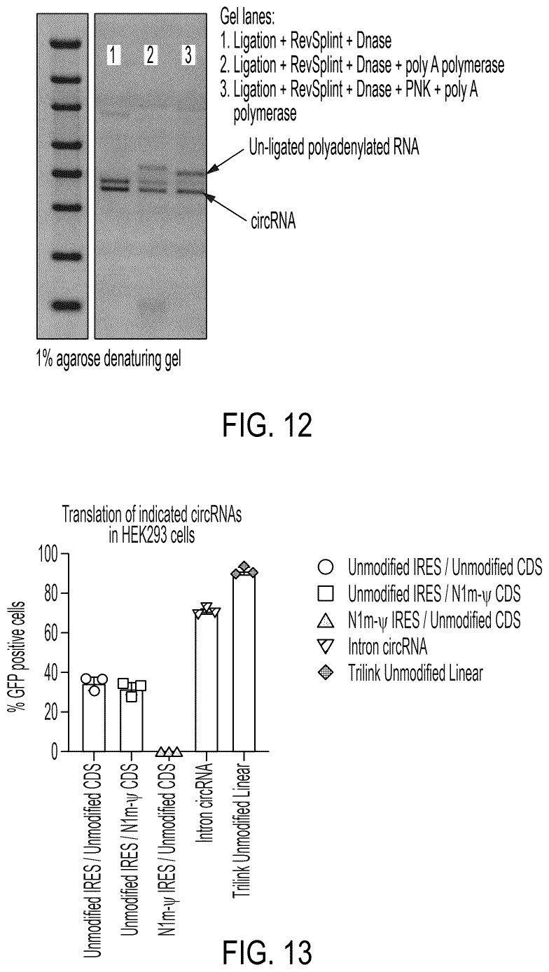

After incubating with ligase I, in addition to removing the reverse splint described in Example 5, the reaction was further treated with a polyA polymerase, which added a poly (A) tail to a free 3′OH of an un-ligated RNA, while the circular RNA was not polyadenylated due to the absence of a 3′-OH end. FIG. 12 is a gel image showing efficient polyadenylation of the linear, un-ligated RNA species in the presence of poly A polymerase. PNK treatment further enhanced the polyadenylation of the un-ligated RNA molecule.

The poly(A) tailed un-ligated RNA was removed using oligo-dT, e.g., a dT-conjugated resin.

### Example 7: Expression of Circular RNA Including an Unmodified or Modified Internal Ribosome Entry Site (IRES)

The flexibility in synthesizing linear RNA fragments separately before ligating them using splint fragments described herein provided the opportunity to test different modifications on IRES and/or coding sequences.

Circular RNAs were generated using a ligation reaction with DNA splint fragments and RNA ligase.

Two linear RNA sequences, an IRES element (modified or unmodified) and a coding sequence (modified or unmodified), were synthesized in vitro, for example, using in vitro transcription (IVT). The two linear RNA sequences (IRES and coding sequences) formed a linear RNA molecule using a first DNA splint with an RNA ligase. The IRES-coding sequence that encodes a GFP reporter was purified using RP-HPLC and circularized using a second DNA splint with an RNA ligase. The circular RNAs were transfected into HEK293 cells and tested for GFP expression in the transfected cells by FACS 24 hours after transfection. As shown in FIG. 13, chimeric circular RNAs comprising unmodified IRES expressed GFP. No GFP expression from chimeric circular RNAs comprising modified IRES was observed, indicating that modification of IRES (e.g., IRES modified with N1m-ψ) inhibited the expression of the protein encoded by the circular RNA.

### Example 8: Immunogenicity of 100% N1m-Ψ Circular RNAs in Mice

In this example, 7-10 weeks old CD1 mice were administered with circular RNAs made with 100% N1m-Ψ or unmodified sequences. Circular RNAs made with a scrambled IRES (non-functional) were administered to mice as a control to compare unmodified and 100% N1m-ψ modified circular RNAs. Each circular RNA molecule was administered to the tail vein via intravenous injection (IV) at 4.5 μg/mouse and 100 μl/mouse. The study design is shown in Table 3.

TABLE 3
Study Design

| Group # | |
|---|---|
| (N = 3) | Circular RNA |
| 1 | CVB3 Unmodified circ_hEPO |
| 2 | |
| 3 | Scrambled Unmodified circ_hEPO |
| 4 | |
| 5 | Scrambled N1mΨ |
| 6 | circ_hEPO |
| 7 | Linear 5moU |
| 8 | lin_hEPO |
| 9 | Linear unmodified (FLuc) |
| 10 | |
| 11 | Vehicle |

The blood samples were collected 6 hours post-injection and subjected to serum cytokine profiling. Blood and tissue (liver and spleen) samples were collected 6 hours post-injection for serum cytokine profiling and qPCR to assess the durability of circular RNA.

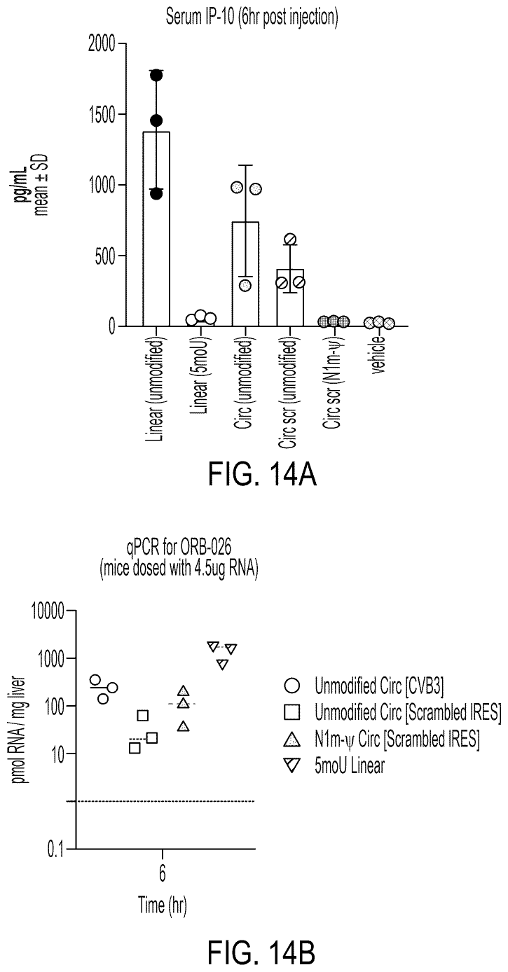

Serum cytokine IP-10 levels 6 hours post-injection were elevated in unmodified circular RNAs but substantially reduced in 100% modified circular RNAs, as shown in FIG. 14A. The results indicated that unmodified uridines in circular RNA may have caused an immune response in mice, while replacement of the uridines with N1m-ψ substantially reduced the immune response in mice (FIG. 14A).

Liver samples collected 6 hours post-injection were also assayed for circular RNA abundance using qPCR assay. As shown in FIG. 14B, circular RNAs made with 100% N1m-w modification tended to be more abundant in mice compared to the unmodified circular RNAs.

### EQUIVALENTS AND SCOPE

While several inventive embodiments have been described and illustrated herein, those of ordinary skill in the art will readily envision various other means and/or structures for performing the function and/or obtaining the results and/or one or more of the advantages described herein. Each of such variations and/or modifications is deemed within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of examples only and that, within the scope of the appended claims and equivalents thereto, inventive embodiments may be practiced otherwise than as specifically described and claimed. Inventive embodiments of the present disclosure are directed to each feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

What is claimed is:

1. A method of making circular RNA, the method comprising:
(i) contacting a linear RNA comprising a 5′ end and a 3′ end with a RNA ligase and a DNA splint comprising a region complementary to the 5′ end of the linear RNA and a region complementary to the 3′ end of the linear RNA, wherein the DNA splint binds the linear RNA and the ligase joins the 5′ end and the 3′ end; and
(ii) removing the DNA splint;
thereby generating circular RNA.

2. A method of joining two linear RNA molecules by ligating with a DNA splint, the method comprising:
(i) contacting the two linear RNA molecules, each comprising a 5′ end and a 3′ end with a RNA ligase and a DNA splint comprising a first region complementary to the 3′ end of the first linear RNA and a second region complementary to the 5′ end of the second linear RNA, wherein the DNA splint binds the linear RNA and the ligase joins the 3′ end of the first linear RNA and the 5′ end of the second linear RNA; and
(ii) removing the DNA splint;
thereby generating a single linear RNA.

3. The method of claim 1 or 2, wherein the linear RNA and the DNA splint are contacted at a 1:1 ratio, a 1:0.5 ratio, or a 1:2 ratio.

4. The method of any one of the preceding claims, wherein the DNA splint is biotinylated.

5. The method of any one of the preceding claims, wherein the DNA splint is single-stranded.

6. The method of any one of the preceding claims, wherein the DNA splint comprises a gap.

7. The method of any one of the preceding claims, wherein the gap is between 1-20, 2-15, or 2-10, or 2-6 nucleotides in length, and wherein the splint anneals to the RNA to form overhangs comprising at least 1-20 non-complementary nucleotides at the 5′ and/or 3′ ends.

8. The method of any one of claims 1-5, wherein the DNA splint comprises no gap, and wherein the splint anneals to the RNA to form overhangs comprising at least 1-20 non-complementary nucleotides at the 5′ and/or 3′ ends.

9. The method of any one of the preceding claims, wherein the gap is 4 nucleotides in length.

10. The method of any of the preceding claims, wherein the gap comprises adenine, thymine, uridine, guanine and/or cytosine nucleotides.

11. The method of any one of the preceding claims, wherein the gap is a homopolymeric stretch of nucleotides.

12. The method of claim 10, wherein the nucleotide is adenine.

13. The method of claim 10, wherein the nucleotide is thymine.

14. The method of claim 10, wherein the nucleotide is guanine.

15. The method of claim 10, wherein the nucleotide is cytosine.

16. The method of claim 10, wherein the nucleotide is uridine.

17. The method of any one of the preceding claims, wherein the gap is comprised of one or more modified nucleotides and/or abasic sites.

18. The method of any one of the preceding claims, wherein the ligase is an RNA ligase.

19. The method of claim 18, wherein the ligase is T4 RNA ligase 1.

20. The method of claim 18, wherein the ligase is T4 RNA ligase 2.

21. The method of claim 18, wherein the ligase is T4 RNA ligase 2 truncated KQ.

22. The method of any one of the preceding claims, wherein a region complementary to the 5′ end of the linear RNA and/or to the 3′ end of the linear RNA is at least 50 nucleotides in length.

23. The method of claim 22, wherein the region complementary to the 5′ end of the linear RNA and/or to the 3′ end of the linear RNA is between 10 to 500 nucleotides in length.

24. The method of claim 22, wherein the region complementary to the 5′ end of the linear RNA and/or to the 3′ end of the linear RNA is between 20 to 120 nucleotides in length.

25. The method of any one of the preceding claims, wherein the region complementary to the 5′ end of the linear RNA and/or to the 3′ end of the linear RNA is between 1% to 10% of the length of the linear RNA.

26. The method of any one of the preceding claims, wherein the region complementary to the 5′ end of the linear RNA and/or to the 3′ end of the linear RNA has a 5′ overhang and/or a 3′ overhang.

27. The method of claim 26, wherein the 5′ overhang is between 2-20 or 2-20 nucleotides in length.

28. The method of claim 26, wherein the 3′ overhang is between 2-20 or 2-10 nucleotides in length.

29. The method of claim 1 or 2, wherein step (ii) comprises digesting with a DNA nuclease.

30. The method of claim 29, wherein the DNA nuclease is a DNA exonuclease.

31. The method of claim 29, wherein the DNA nuclease is DNase I.

32. The method of claim 4, wherein step (ii) comprises denaturing and binding a biotinylated splint to streptavidin.

33. The method of claim 1 or 2, wherein step (ii) comprises annealing the DNA splint with a reverse splint and digesting with a DNA nuclease.

34. The method of claim 33, wherein the DNA nuclease is a DNA exonuclease.

35. The method of claim 33, wherein the DNA nuclease is DNase I.

36. The method of claim 4, wherein step (ii) comprises denaturing and binding a biotinylated reverse splint to streptavidin.

37. The method of claim 1 or 2, wherein step (ii) comprises annealing the splint with a reverse splint comprising a homopolymer of 10-50 adenine nucleotides, thereby forming splint:reverse splint duplex that is removed by a homopolymer of thymine nucleotides.

38. The method of claim 1 or 2, wherein the splint contains a homopolymer of 10-50 adenines removed by a homopolymer of thymine nucleotides after ligation.

39. The method of any one of the preceding claims, wherein the method further comprises digesting the un-ligated RNA with an RNA exonuclease.

40. The method of claim 39, wherein the RNA nuclease is RNase R.

41. The method of claim 39, wherein the RNA nuclease is Xrn1.

42. The method of any one of claims 1-39, wherein the method further comprises treating the ligation reaction with a polyA polymerase to polyadenylate un-ligated RNA, thereby removing the poly(A)+ RNA from the reaction using a homopolymer of thymine nucleotides.

43. The method of claim 42, wherein the poly A polymerase is Escherichia coli poly A polymerase.

44. The method of claim 42, wherein the poly A polymerase is yeast poly A polymerase.

45. The method of any one of the preceding claims, wherein the linear RNA is synthetic RNA.

46. The method of any one of the preceding claims, wherein the linear RNA is in vitro transcribed RNA.

47. The method of claim 46, wherein the in vitro transcription is in the presence of GMP.

48. The method of claim 46, wherein the in vitro transcribed RNA is treated with phosphatase.

49. The method of claim 48, wherein the in vitro transcribed RNA is treated with kinase.

50. The method of claim 46, wherein RppH converts triphosphate to monophosphate.

51. The method of any one of the preceding claims, wherein the 5′ end is a 5′ monophosphate.

52. The method of any one of the preceding claims, wherein the 5′ end is pre-adenylated.

53. The method of any one of the preceding claims, wherein the 3′ end is a 3′ hydroxyl.

54. The method of any one of the preceding claims, wherein the linear RNA is messenger RNA.

55. The method of any one of the preceding claims, wherein the linear RNA and/or circular RNA comprises one or more modified nucleotides.

56. The method of claim 55, wherein the modified nucleotide is one or more of N1-methylpseudouridine, 5-methoxyuridine, N6-methyladenosine, pseudouridine or 5-methylcytosine.

57. The method of claim 56, wherein the modified nucleotide is N1-methylpseudouridine.

58. The method of claim 56, wherein the modified nucleotide is 5-methoxyuridine.

59. The method of claim 56, wherein the modified nucleotide is N6-methyladenosine.

60. The method of claim 56, wherein the modified nucleotide is pseudouridine.

61. The method of claim 56, wherein the modified nucleotide is 5-methylcytosine.

62. The method of any one of claims 55 to 61, wherein all the nucleotides of the circular RNA or linear RNA are modified nucleotides.

63. The method of any one of claims 55 to 61, wherein less than 50% of the nucleotides of the circular RNA or linear RNA are modified nucleotides.

64. The method of any one of claims 55 to 61, wherein less than 20% of the nucleotides of the circular RNA or linear RNA are modified nucleotides.

65. The method of any one of claims 55 to 61, wherein less than 10% of the nucleotides of the circular RNA or linear RNA are modified nucleotides.

66. The method of any one of the preceding claims, wherein the linear RNA and/or circular RNA is between 100 base pairs to 10 kilobases in length.

67. The method of claim 66, wherein the linear RNA and/or circular RNA is between 100 base pairs to 500 base pairs in length.

68. The method of claim 66, wherein the linear RNA and/or circular RNA is between 500 base pairs to 10 kilobases in length.

69. The method of any one of the preceding claims, wherein the linear RNA and/or circular RNA encodes a therapeutic protein.

70. The method of any one of the preceding claims, wherein the linear RNA and/or circular RNA is an RNA vaccine.

71. A circular RNA is generated by the method of any one of the preceding claims.

72. The circular RNA of claim 71, wherein the modified nucleotide is N1-methylpseudouridine or 5-methoxyuridine.

73. The circular RNA of claim 72, wherein the modified nucleotide is N1-methylpseudouridine.

74. The circular RNA of claim 72, wherein the modified nucleotide is 5-methoxyuridine.

75. The circular RNA of any one of claims 72 to 74, wherein the modified nucleotide is 100% of the overall nucleotide content.

76. The circular RNA of any one of claims 72 to 74, wherein the modified nucleotide is less than 50% of the overall nucleotide content.

77. The circular RNA of any one of claims 72 to 74, wherein the modified nucleotide is less than 20% of the overall nucleotide content.

78. The circular RNA of any one of claims 72 to 74, wherein the modified nucleotide is less than 10% of the overall nucleotide content.

79. A method of manufacturing RNA comprising a step of circularizing RNA by the method of any one of the preceding claims.

80. A method of making circular RNA, the method comprising:
(i) joining two linear RNA molecules with a DNA splint, according to claim 2, to form a linear RNA precursor comprising a 5′ end and a 3′ end;
(ii) contacting the linear RNA precursor with a RNA ligase and a DNA splint comprising a region of homology to the 5′ end of the linear RNA precursor and a region of homology to the 3′ end of the linear RNA precursor, wherein the DNA splint binds the linear RNA and the ligase joins the 5′ end and the 3′ end, thereby generating circular RNA.

81. A method of making circular RNA, the method comprising:
(i) contacting at least two linear RNA molecules, each comprising a 5′ end and a 3′ end with an RNA ligase, and at least two DNA splints,
wherein the first DNA splint comprises two regions of homology to the 3′ end of the first linear RNA and the 5′ end of the second linear RNA,
wherein the second DNA splint comprises two regions of homology to the 3′ end of the second linear RNA and the 5′ end of the first linear RNA,
such that the two DNA splints bind the linear RNA molecules and the ligase joins the ends of the linear RNA molecules; and
(ii) removing the DNA splints;
thereby generating a circular RNA.

82. The method of claim 80 or 81, wherein more than two linear RNA molecules are joined by annealing DNA splints to form a linear RNA precursor to generate the circular RNA.

83. The method of claim 81, wherein the two or more linear RNAs, RNA ligase, and the two or more DNA splints are mixed in one solution, generating the circular RNA.

84. The method of any one of claims 80-83, wherein the at least two linear RNA molecules comprise an Internal Ribosome Entry Site (IRES) element, a coding polynucleotide that encodes a polypeptide of interest, a microRNA binding site, a microRNA, an inhibitory RNA sequence, an active RNA sequence, a 3′ untranslated region (3′ UTR), a 5′ untranslated region (5′ UTR), an aptamer, and/or a signal sequence.

85. The method of any one of claims 80-84, wherein the at least two linear RNAs comprise a coding sequence.

86. The method of any one of claims 80-85, wherein the first linear RNA is an Internal Ribosome Entry Site (IRES) element, and the second linear RNA is a messenger RNA that encodes a protein.

87. The method of any one of claims 80-86, wherein the first linear RNA is not modified.

88. The method of any one of claims 80-87, wherein the first linear RNA is chemically modified.

89. The method of any one of claims 80-88, wherein the second linear RNA is chemically modified.

90. The method of claim 89, wherein the second linear RNA is modified with 1-methylpseudouridine (N1m-ψ), 5-methoxyuridine (5moU), N6-methyladenosine (m6A), pseudouridine (ψ) or 5-methylcytosine (m5C).

91. The method of any one of claims 89-90, wherein the modification is 100%.

92. The method of any one of claims 89-91, wherein the modification is 80-50%, or 60%-20%, or 50%-10%, or 20%-10%.

93. The method of any one of claims 80-91, the method further comprising purifying the circular RNA using HPLC, SEC, RNase R treatment, and/or poly(dT)-conjugated resin.

94. A circular RNA molecule comprising an internal ribosome entry site (IRES) operably linked to at least one coding polynucleotide that encodes a protein of interest, wherein the IRES lacks N1-methylpseudouridine (N1m-ψ) modification.

95. A circular RNA comprising an internal ribosome entry site (IRES) operably linked to at least one coding polynucleotide that encodes a protein of interest, wherein the IRES comprises about 1-10% modified nucleotides.

96. A method of expressing a protein of interest in a cell comprising contacting the cell with the circular RNA of claim 94 or 95.
